(19) United States
(12) Patent Application Publication
(10) Pub. No.: US 2024/0000949 A1
(43) Pub. Date: Jan. 4, 2024

(54) Nucleic Acid-Derivatized Therapeutics

(71) Applicant: The University of Chicago, Chicago, IL (US)

(72) Inventors: Lev BECKER, Chicago, IL (US); Yamuna KRISHNAN, Chicago, IL (US); Chang CUI, Chicago, IL (US); Kasturi CHAKRABORTY, Chicago, IL (US)

(21) Appl. No.: 18/042,958

(22) PCT Filed: Sep. 7, 2021
(86) PCT No.: PCT/US2021/049306

§ 371 Date: Feb. 24, 2023

Related U.S. Application Data
(60) Provisional application No. 63/074,644, filed on Sep. 4, 2020.

(51) Int. Cl. A61K 47/54; A61K 45/06; A61K 38/00; A61K 31/5377; A61K 31/52; A61K 31/195; A61K 31/145; A61P 3/06; A61P 35/00

(52) U.S. Cl. CPC A61K 47/549; A61K 45/06; A61K 38/005; A61K 31/5377; A61K 31/52; A61K 31/195; A61K 31/145; A61P 3/06; A61P 35/00

(57) ABSTRACT

This disclosure relates to nucleic acid-derivatized therapeutics and methods of their use.

### CROSS-REFERENCE TO RELATED APPLICATIONS

This application is the U.S. national phase under 35 U.S.C. § 371 of International Application No. PCT/US2021/049306, filed Sep. 7, 2021, which claims priority to U.S. Provisional Application No. 63/074,644, filed Sep. 4, 2020, which are incorporated herein by reference in their entirety.

### SEQUENCE LISTING STATEMENT

A computer readable form of the Sequence Listing is filed with this application by electronic submission and is incorporated into this application by reference in its entirety. The Sequence Listing is contained in the file created on Aug. 30, 2023, having the file name “19-1734-WO-US_ST25.txt” and is 19,290 bytes in size.

### BACKGROUND OF DISCLOSURE

### Field of Invention

This disclosure relates to nucleic acid-derivatized therapeutics and methods of use thereof.

### Technical Background

### Macrophages

Macrophages are the most plastic cells of the hematopoietic system and found in most if not all tissues in various forms (e.g., histiocytes, Kupffer cells, alveolar macrophages, microglia, etc.). With the ultimate goal of maintaining homeostasis, tissue macrophages acquire unique transcriptional profiles and functional capabilities specifically and dynamically tailored to their environment. For instance, during early stages of infection, macrophages recognize and destroy a wide range of pathogens. They secrete pro-inflammatory cytokines and/or present antigens to alert the adaptive immune system. During wound healing and tissue repair, macrophages adopt an immunosuppressive state. They secrete anti-inflammatory cytokines and suppress the adaptive immune response. In response to nutrient excess, macrophages phagocytose and digest lipids to maintain adipose tissue and liver metabolic homeostasis. Thus, through their ability to kill pathogens, phagocytose debris, and instruct other cell types, macrophages play a central role in clearing infections and maintaining homeostasis. However, their homeostatic functions can be subverted by imbalanced environmental signals/chronic insults, resulting in a causal association of macrophages with many diseases including cancer, atherosclerosis, obesity/type 2 diabetes, asthma, arthritis, and susceptibility to infections.

In cancer, tumor-associated macrophages (TAMs) are the most prevalent immune cells in the tumor microenvironment. TAMs mainly adopt an M2-like immunosuppressive phenotype. They overexpress growth factors (e.g., VEGFa) that promote angiogenesis, secrete proteases (e.g., MMPs) that facilitate metastatic dissemination and produce inhibitory molecules (e.g., ARG1, IL10, and PD-L1) that suppress adaptive immune responses. Depleting TAMs in pre-clinical models attenuated tumor growth and metastasis, and high TAM abundance in human tumors correlates with poor survival in patients across many cancer types. For these reasons, M2-like TAMs are an emerging target for anti-cancer therapy development.

During obesity/Type 2 Diabetes (T2D), macrophages accumulate in visceral adipose tissue where they promote a chronic state of low-grade inflammation that has been causally associated with insulin resistance in mice. Inhibiting pathways that drive inflammatory cytokine production and/or signaling improves insulin sensitivity. Studies showed that during obesity, adipose tissue macrophages (ATMs) adopt a metabolically activated (MMe) macrophage phenotype, which is distinct from the pro-inflammatory M1 phenotype that predominates during infection. Hence, understanding the dynamic regulation of ATMs is essential to specifically target pro-inflammatory pathway in obesity/T2D without affecting the ability of macrophages to fight infections.

In coronary heart disease, macrophages have been causatively linked to initiation, progression, and rupture of atherosclerotic plaques. Their inability to clear cholesterol leads to the formation of foam cells, a type of macrophage that localizes to fatty deposits on blood vessel walls and ingest low-density lipoproteins (thus assuming a “foamy” appearance). Furthermore, their defective clearance of apoptotic cells in the artery wall promotes necrotic core formation and increases plaque complexity, and their increased secretion of proteases destabilizes atherosclerotic plaques and promotes plaque vulnerability. Accordingly, macrophages are an attractive cellular target for therapies aimed at treating coronary heart disease.

Considering the abundance and heterogeneity of macrophages, it is not surprising that macrophages play an integral role in maintaining tissue homeostasis and are involved in many pathophysiological mechanisms. Because they exhibit a wide spectrum of pro-inflammatory, destructive, immunosuppressive, and remodeling capabilities in different disease settings, therapeutics that are tailored to precisely target macrophages or a specific subcellular compartment within them have great potential.

### Lysosomes

Lysosomes are ubiquitous organelles that constitute the primary degradative compartments of the cell. They receive their substrates through endocytosis, phagocytosis, pinocytosis, or autophagy. Two classes of proteins are essential for the function of lysosomes: soluble lysosomal hydrolases (also referred to as acid hydrolases) and integral lysosomal membrane proteins (LMPs). Each of the 50 known lysosomal hydrolases targets specific substrates for degradation, and their collective action is responsible for the total catabolic capacity of the lysosome. In addition to bulk degradation and pro-protein processing, lysosomes are involved in degradation of the extracellular matrix, initiation of apoptosis, and antigen processing.

### Scavenger Receptors

Scavenger receptors constitute a heterogeneous family of receptors capable of recognizing and binding to a broad spectrum of ligands, including modified and unmodified host-derived molecules (through damage-associated molecular patterns, or DAMPs) in addition to microbial components (through pathogen-associated molecular patterns, or PAMPs). These ligands can constitute a variety of polyanionic binding partners, including lipoproteins, apoptotic cells, cholesterol esters, phospholipids, proteoglycans, ferritin, carbohydrates, and nucleic acids.

The receptors are incredibly diverse and organized into many different classes, starting at A and continuing to L—an organization that is based on their structural properties. However, there is little or no sequence homology between the classes, and the superfamily grouping is purely a consequence of shared functional properties. Due to the significant diversity within the family and continuing research into scavenger receptor structure and function, the receptors lack an accepted nomenclature and have been described under several different naming systems.

Scavenger receptors function in a wide range of biological processes, such as endocytosis, adhesion, lipid transport, antigen presentation, and pathogen clearance. In addition to playing a crucial role in maintenance of host homeostasis, scavenger receptors have been implicated in the pathogenesis of a number of diseases, e.g., atherosclerosis, neurodegeneration, or metabolic disorders. Additionally, these receptor molecules are also important regulators of tumor behavior and host immune responses to cancer.

Scavenger receptors are expressed primarily on dendritic cells, endothelial cells, and macrophages. Specific classes of the receptors exhibit characteristic expression patterns on specific cell types—for instance, Class A receptors are expressed primarily on tissue macrophages and macrophage subtypes, such as Kupffer cells, and cortical and medullary thymic macrophages. The expression of scavenger receptors is significantly higher on macrophages over their precursors, monocyte cells.

### Targeted Drug Delivery to Macrophages

The ability to reprogram macrophages in vivo depends on a robust cellular targeting strategy to selectively deliver therapeutics to macrophages. Several carrier technologies have been developed for preferentially targeting macrophages. These include nanoparticles such as liposomes and microspheres and antibody-drug conjugates (ADCs). Nanoparticles can target macrophages passively via their high phagocytic potential or actively, by decorating them with mannose (binds CD206 on macrophages) or galactose-type lectin I (binds asialoglycoprotein receptor on macrophages). However, nanoparticle-based systems interact with other innate immune cells beyond macrophages and thus have poor selectivity. ADCs using anti-CD206 (binds CD206 on macrophages) or Fc (binds Fcgγ receptor on macrophages) have also been employed. While these approaches have improved selectivity, problems associated with low efficiency of drug internalization have been reported. Moreover, these approaches are challenged by difficulties in obtaining defined conjugation ratios and in delivering multiple drugs in combination. Therefore, there is a need for new approaches to selectively deliver drugs in controllable stoichiometries to the same location/cell type to macrophages within the body.

### SUMMARY OF THE DISCLOSURE

This disclosure describes nucleic acid-derivatized therapeutics and methods of their use. As described below, in one aspect, the disclosure provides a composition, comprising a nucleic acid targeting module and a therapeutic agent attached to the nucleic acid targeting module, wherein the nucleic acid targeting module targets the therapeutic agent to a lysosome of a macrophage.

In some embodiments of the first aspect, the therapeutic agent is covalently attached to the nucleic acid targeting module. In some embodiments of the first aspect, the nucleic acid targeting module comprises single stranded deoxyribose nucleic acid (ssDNA), double-stranded DNA (dsDNA), modified DNA, single stranded ribonucleic acid (ssRNA), double-stranded RNA (dsRNA), modified RNA, and/or a RNA/DNA complex. In some embodiments of the first aspect, the nucleic acid targeting module is a double-stranded DNA molecule. In some embodiments of the first aspect, the nucleic acid targeting module is 38 base pairs in length.

In some embodiments of the first aspect, the nucleic acid targeting module comprises a first single-stranded nucleic acid molecule and a second single-stranded nucleic acid molecule that is partially or fully complementary to the first single-stranded molecule. In some of these embodiments of the first aspect, each of the first and second single-stranded nucleic acid molecules is between 15 and 500 nucleotides in length. In some of these embodiments of the first aspect, each of the first and second single-stranded nucleic acid molecules is between 30 and 50 nucleotides in length. In some of these embodiments of the first aspect, the first single-stranded nucleic acid molecule comprises the nucleic acid sequence of SEQ ID NO: 40. In some of these embodiments of the first aspect, the second single-stranded nucleic acid molecule comprises the nucleic acid sequence of SEQ ID NO: 41 or SEQ ID NO: 42. In some of these embodiments of the first aspect, the therapeutic agent is covalently attached to the first and/or second single-stranded nucleic acid molecule.

In some embodiments of the first aspect, the therapeutic agent comprises a small molecule. In some embodiments of the first aspect, the therapeutic agent comprises a peptide.

In some embodiments of the first aspect, the therapeutic agent comprises a cathepsin inhibitor, a LDHA inhibitor, a neoantigen, a BTK inhibitor, a SYK inhibitor, and/or an LXR agonist. In some of these embodiments of the first aspect, the cathepsin inhibitor is a cysteine protease inhibitor or an aspartic protease inhibitor. In some of these embodiments, the cysteine protease inhibitor is E64. In some of these embodiments, the aspartic protease inhibitor is CA074 and/or pepstatin A. In some of these embodiments of the first aspect, the LDHA inhibitor is FX11, gossypol, GSK2837808A, (R)-GNE-140, galloflavin, NHI-2, and/or machilin. In some of these embodiments of the first aspect, the BTK inhibitor is ibrutinib. In some of these embodiments of the first aspect, the LXR agonist is GW3965 and/or T0901317.

In some embodiments of the first aspect, the composition further comprises a labeling module optionally attached to the nucleic acid targeting module and/or the therapeutic agent. In some of these embodiments of the first aspect, the labeling module comprises one or more of a fluorescent agent, a chemiluminescent agent, a chromogenic agent, a quenching agent, a radionucleotide, an enzyme, a substrate, a cofactor, an inhibitor, a nanoparticle, and a magnetic particle.

In some embodiments of the first aspect, the composition further comprises a pharmaceutically acceptable carrier, a solvent, an adjuvant, a diluent, or a combination thereof.

In a second aspect, the disclosure provides a method of treating or preventing cancer in a subject in need thereof, comprising administering to the subject a composition, the composition comprising a nucleic acid targeting module and one or more therapeutic agents.

In some embodiments of the second aspect, at least one of the one or more therapeutic agents is attached to the nucleic acid targeting module. In some embodiments of the second aspect, the nucleic acid targeting module targets the one or more therapeutic agents to a lysosome of a tumor associated macrophage (TAM). In some embodiments of the second aspect, the one or more therapeutic agents comprises one or more of a cathepsin inhibitor, an LDHA inhibitor, and a neoantigen. In some embodiments of the second aspect, the nucleic acid targeting module preferentially targets M2-like TAMs. In some of these embodiments of the second aspect, the method further comprises reducing the lysosomal degradative capacity of the TAM. In some of these embodiments of the second aspect, the method further comprises increasing cancer-derived antigen presentation by the TAM.

In some embodiments of the second aspect, the method further comprises increasing intratumoral activated CD8⁺ cytotoxic T lymphocyte (optionally CD45⁺, CD3⁺, CD8⁺, CD62L⁻, and/or CD44⁺) populations in the subject. In some embodiments of the second aspect, the method further comprises increasing T-cell activation and proliferation. In some embodiments of the second aspect, the method further comprises functionalizing CD8+ T cells. In some embodiments of the second aspect, the method further comprises reducing tumor volume in the subject. In some embodiments of the second aspect, the method slows the growth of one or more tumors. In some embodiments of the second aspect, the method further comprises administering an immune checkpoint inhibitor to the subject. In some of these embodiments, the immune checkpoint inhibitor is an anti-PD-L1 antibody, an anti-PD-1 antibody, an anti-CTLA-4 antibody, an anti-LAG-3 antibody, an anti-TIM-3 antibody, an anti-TIGIT antibody, an anti-B7-H3 antibody, an anti-VISTA antibody, an anti-CD47 antibody, or combinations thereof.

In some embodiments of the second aspect, the cancer is breast cancer, colorectal cancer, lung cancer, ovarian cancer, pancreatic adenocarcinoma, pancreatic neuroendocrine cancer, osteosarcoma, or melanoma. In some embodiments of the second aspect, the method further comprises administering a BTK inhibitor to the subject.

In a third aspect, the disclosure provides a method of treating obesity in a subject in need thereof, comprising administering to the subject a composition, the composition comprising a nucleic acid targeting module and one or more therapeutic agents attached to the nucleic acid targeting module, wherein the nucleic acid targeting module targets the one or more therapeutic agents to a lysosome of a macrophage.

In a fourth aspect, the disclosure provides a method of treating diabetes in a subject in need thereof, comprising administering to the subject a composition, the composition comprising a nucleic acid targeting module and one or more therapeutic agents attached to the nucleic acid targeting module, wherein the nucleic acid targeting module targets the one or more therapeutic agents to a lysosome of a macrophage.

In a fifth aspect, the disclosure provides a method of treating insulin resistance in a subject in need thereof, comprising administering to the subject a composition, the composition comprising a nucleic acid targeting module and one or more therapeutic agents attached to the nucleic acid targeting module, wherein the nucleic acid targeting module targets the one or more therapeutic agents to a lysosome of a macrophage.

In some embodiments of the third, fourth, or fifth aspects, the one or more therapeutic agents comprises one or more of a BTK inhibitor and a SYK inhibitor. In some embodiments of the third, fourth, or fifth aspects, the BTK inhibitor comprises ibrutinib.

In a sixth aspect, the disclosure provides a method of treating atherosclerosis in a subject in need thereof, comprising administering to the subject a composition, the composition comprising a nucleic acid targeting module and an LXR agonist attached to the nucleic acid targeting module, wherein the nucleic acid targeting module targets the LXR agonist to the lysosome of a macrophage.

In a seventh aspect, the disclosure provides a composition, comprising a DNA targeting platform comprising a dsDNA targeting module and a cathepsin inhibitor, and a secondary therapeutic agent. In some embodiments of the seventh aspect, the secondary therapeutic agent is an immune checkpoint inhibitor. In some of these embodiments, the immune checkpoint inhibitor is an anti-PD-L1 antibody or an anti-CD47 antibody. In some embodiments of the seventh aspect, the secondary therapeutic agent is attached to the DNA targeting platform. In some embodiments of the seventh aspect, the secondary therapeutic agent comprises one or more of daunorubicin, vincristine, epirubicin, idarubicin, valrubicin, mitoxantrone, paclitaxel, docetaxel, cisplatin, camptothecin, irinotecan, 5-fluorouracil, methotrexate, dexamethasone, and cyclophosphamide. In some of these embodiments, the secondary therapeutic agent is cyclophosphamide. In some of these embodiments of the seventh aspect, the dsDNA targeting module comprises the nucleic acid sequence of SEQ ID NO: 40 and the nucleic acid sequence of SEQ ID NO: 41 or SEQ ID NO: 42, the cathepsin inhibitor is E64, and the secondary therapeutic agent is cyclophosphamide. In some embodiments of the seventh aspect, the secondary therapeutic agent is a neoantigen.

In an eighth aspect, the disclosure provides a composition, comprising a DNA targeting platform, comprising a dsDNA targeting module and one or more of a cathepsin inhibitor, an LDHA inhibitor, and a neoantigen.

In a ninth aspect, the disclosure provides a composition, comprising a DNA targeting platform comprising a dsDNA targeting module and one or more of a BTK inhibitor and a SYK inhibitor.

In a tenth aspect, the disclosure provides a composition, comprising a DNA targeting platform comprising a dsDNA targeting module and an LXR agonist.

In some embodiments of the first, eighth, ninth, or tenth aspect, the composition further comprises a secondary therapeutic agent. In some embodiments of the first, eighth, ninth, or tenth aspect, the composition is formulated for intratumoral administration. In some embodiments of the first, eighth, ninth, or tenth aspect, the composition is formulated for intravenous administration.

In an eleventh aspect, the disclosure provides a method of administering a therapeutic agent to a subject, comprising providing a therapeutic construct comprising a therapeutic agent attached to a nucleic acid targeting module, wherein the nucleic acid targeting module targets the therapeutic agent to a lysosome of a macrophage, and administering the therapeutic construct to the subject.

In a twelfth aspect, the disclosure provides a method, comprising administering to a subject a therapeutic construct comprising a therapeutic agent attached to a nucleic acid targeting module, wherein the nucleic acid targeting module targets the therapeutic agent to a lysosome of a macrophage.

In some embodiments of the eleventh or twelfth aspect, the therapeutic agent is released from the lysosome of the macrophage upon degradation of the nucleic acid targeting module.

In a thirteenth aspect, the disclosure provides a method of minimizing a side-effect of a therapeutic agent, comprising administering to a subject in need thereof a therapeutic agent attached to a nucleic acid targeting module, wherein the nucleic acid targeting module targets the therapeutic agent to a lysosome of a macrophage, wherein the therapeutic agent is released from the lysosome of the macrophage upon degradation of the targeting module, wherein the therapeutic agent is released into the cytosol, nucleus, and/or immediate extracellular microenvironment of the macrophage to minimize the side-effect of the therapeutic agent that occurs when the therapeutic agent administered systemically.

In some embodiments of the eleventh, twelfth, and thirteenth aspects, the therapeutic agent comprises a small molecule. In some embodiments of the eleventh, twelfth, and thirteenth aspects, the therapeutic agent comprises a peptide.

In a fourteenth aspect, the disclosure provides a method of sensitizing a subject to a therapy, comprising administering to a subject a therapeutic construct comprising a therapeutic agent attached to a nucleic acid targeting module, wherein the nucleic acid targeting module targets the therapeutic agent to a lysosome of a macrophage, and administering to the subject the therapy to which the subject is to be sensitized. The therapeutic construct sensitizes the subject to the therapy. In some embodiments of the fourteenth aspect, the therapy to which the subject is to be sensitized is an immune checkpoint inhibitor therapy. In some of these embodiments of the fourteenth aspect, the immune checkpoint inhibitor therapy comprises an anti-PD-L1 antibody, an anti-PD-1 antibody, an anti-CTLA-4 antibody, an anti-LAG-3 antibody, an anti-TIM-3 antibody, an anti-TIGIT antibody, an anti-B7-H3 antibody, an anti-VISTA antibody, an anti-CD47 antibody, or combinations thereof. In some embodiments, the immune checkpoint inhibitor therapy is an anti-PD-L1 antibody. In some embodiments of the fourteenth aspect, the therapeutic agent attached to the nucleic acid targeting module is E64. In some embodiments of the fourteenth aspect, the nucleic acid targeting module is 38 base pairs in length.

In a fifteenth aspect, the disclosure provides a composition, comprising a nucleic acid targeting module and a labeling module attached to the nucleic acid targeting module, wherein the nucleic acid targeting module targets the labeling module to a lysosome of a macrophage. In some embodiments of the fifteenth aspect, the labeling module comprises a contrast agent. In some embodiments of the fifteenth aspect, the contrast agent comprises iron oxide, iron platinum, manganese, and/or gadolinium. In some embodiments of the fifteenth aspect, the labeling module comprises gadolinium.

In a sixteenth aspect, the disclosure provides a method of administering a labeling module to a subject, comprising providing a labeling construct comprising a labeling module attached to a nucleic acid targeting module, wherein the nucleic acid targeting module targets the labeling construct to a lysosome of a macrophage, and administering the labeling construct to the subject.

In a seventeenth aspect, the disclosure provides a method, comprising administering to a subject a labeling construct comprising a labeling module attached to a nucleic acid targeting module, wherein the nucleic acid targeting module targets the labeling module to a lysosome of a macrophage.

In an eighteenth aspect, the disclosure provides a method of imaging a biological phenomenon in a subject, comprising administering to a subject a labeling construct comprising a labeling module attached to a nucleic acid targeting module, wherein the nucleic acid targeting module targets the labeling module to a lysosome of a macrophage, and detecting the labeling module. In some embodiments of the eighteenth aspect, the biological phenomenon is a tumor or atherosclerotic lesion. In some embodiments of the eighteenth aspect, the labeling module comprises iron oxide, iron platinum, manganese, and/or gadolinium. In some embodiments of the eighteenth aspect, the labeling module is detected by magnetic resonance imaging.

In a nineteenth aspect, the disclosure provides a method of imaging a biological phenomenon associated with obesity in a subject in need thereof, comprising administering to the subject a composition, the composition comprising a nucleic acid targeting module and one or more labeling modules attached to the nucleic acid targeting module, wherein the nucleic acid targeting module targets the one or more labeling modules to a lysosome of a macrophage.

In a twentieth aspect, the disclosure provides a method of imaging a biological phenomenon associated with diabetes in a subject in need thereof, comprising administering to the subject a composition, the composition comprising a nucleic acid targeting module and one or more labeling modules attached to the nucleic acid targeting module, wherein the nucleic acid targeting module targets the one or more labeling modules to a lysosome of a macrophage.

In a twenty-first aspect, the disclosure provides a method of imaging a biological phenomenon associated with insulin resistance in a subject in need thereof, comprising administering to the subject a composition, the composition comprising a nucleic acid targeting module and one or more labeling modules attached to the nucleic acid targeting module, wherein the nucleic acid targeting module targets the one or more therapeutic agents to a lysosome of a macrophage.

In some embodiments of the nineteenth, twentieth, and twenty-first aspect, the biological phenomenon is inflammation.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure and are incorporated in and constitute a part of this specification. The drawings illustrate one or more embodiment(s) of the disclosure, and together with the description serve to explain the principles and operation of the disclosure.

FIGS. 1A-1B. Uptake of various oligonucleotides by bone marrow-derived macrophages (BMDMs). FIG. 1A, Schematic of various fluorescently labelled nucleic acid structures used for uptake studies in BMDMs. Each nucleic acid scaffold is either a single stranded or double stranded 38 mer DNA (D) or RNA (R) sequence, or a DNA:RNA hybrid or complex. Each scaffold is labelled with an Alexa Fluor® 647N fluorophore on the 5′ end of one of the strands. From left to right, the constructions tested were dsDNA (SEQ ID NO: 40 and SEQ ID NO: 41 or SEQ ID NO: 42), ssDNA (SEQ ID NO: 41), dsRNA (SEQ ID NO: 43 and SEQ ID NO: 44), ssRNA (SEQ ID NO: 43), and ssDNA:ssRNA (SEQ ID NO: 45 and SEQ ID NO: 46). FIG. 1B, BMDMs were pulsed with 100 nM of each nucleic acid scaffold for 30 min. The cells were then washed and chased for 15 min after which were subjected to flow cytometry based quantification. Mean fluorescence intensity (A/IFI) of nucleic acid scaffold uptake by BMDMs is shown.

FIGS. 2A-2B. dsDNA preferentially targets macrophages in other tissues. FIG. 2A, Fluorescently labeled dsDNA (100 μg) was injected intratracheally into mice and cells were harvested 2 hr post injection with a bronchoalveolar lavage. Uptake by alveolar macrophages (AM, CD45⁺CD11b⁻CD11c⁺) and alveolar neutrophils (AN, CD45⁺CD11b⁺Ly6G⁺) was quantified by flow cytometry. FIG. 2B, Fluorescently labeled dsDNA (100 μg) was injected intraperitoneally into mice and visceral adipose tissue was harvested 4 hr post injection. Adipose tissue was digested to obtain the stromal vascular fraction. dsDNA uptake by cells in the stromal vascular fraction was quantified by flow cytometry. ATM=adipose tissue macrophage (CD45⁺CD11b⁺F4/80⁺).

FIGS. 3A-3C. I.V. delivered E64-DNA traffics to E0771 tumors, but is not internalized by blood cells. FIG. 3A, Experimental design. FIG. 3B, Representative flow images of E64-DNA uptake by blood cells and tumor cells. FIG. 3C, Mean fluorescence intensity (A/IFI) of E64-DNA uptake in blood cells and tumor cells.

FIGS. 4A-4B. A DNA complexed liver X receptor (LXR) agonist (TO-DNA) induces LXR target genes in macrophages. The LXR agonists T0901317 (TO) or GW3965 (GW) were covalently attached to double-stranded DNA. FIG. 4A, Effect of vehicle (control, Ctrl), DNA, TO-DNA, on LXR target gene expression (Apoe, Abcal, Abcgl). Free T0901317 (100 nM) was included as a positive control. n=4/group. FIG. 4B, Effect of vehicle (control, Ctrl), DNA, GW-DNA (100 nM), on LXR target gene expression (Apoe, Abcal, Abcgl). Free GW3965 (100 nM) was included as a positive control. n=4/group. *,p<0.05 Student's t-test.

FIG. 5. Schematic of DNA-based macrophage targeting platform (DNA-based nanodevice).

FIG. 6A-6D. M2 macrophages have elevated lysosomal enzyme levels and activity. FIG. 6A, Shotgun proteomics analysis of whole cell lysates from M1 and M2 BMDMs. Differentially abundant proteins were identified by the G-test and t-test (FDR<5%). n=5/group.

FIG. 6B, Levels of known M1/M2-associated proteins from proteomics data. Proteins were quantified by spectral counting and standardized to the condition with highest abundance. n=5/group. FIG. 6C, Top five pathways from gene ontology (GO) analysis of proteins elevated in M2 BMDMs (p<0.05, Fisher's exact test with Benjamini-Hochberg correction). FIG. 6D, Heatmap of lysosomal protein levels in M1 and M2 BMDMs. Scale: (M2−M1avg)/(M2+M1avg) or (M1−M2avg)/(M1+M2avg). n=5/group. All measurements (n) are biological replicates.

FIG. 7. TFEB is responsible for elevated lysosomal enzymes in M2-like macrophages. Validation of lysosomal proteins elevated in M2 BMDMs by immunoblotting, related to FIG. 6D. Representative of 2 independent experiments.

FIG. 8. M2 macrophages have elevated lysosomal enzyme levels and activity. DQ-OVA degradation assays of M1 and M2 BMDMs. Assay scheme (top) and quantification (bottom). n=3/group.

FIG. 9. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. M1 and M2 activated BMDMs from wild type mice (corresponds to FIG. 8).

FIG. 10. Gating strategy for TAMs. Gating strategy for flow sorting of M1-like and M2-like TAMs from E0771 tumors (corresponds to FIG. 11A).

FIG. 11. M2 macrophages have elevated lysosomal enzyme levels and activity. FIG. 27A, M1-like and M2-like TAMs were sorted from murine E0771 tumors. FIGS. 11B-11C, mRNA levels of M1- and M2-associated genes (FIG. 11B), protein levels of representative M2-like markers and lysosomal proteins by proteomics (FIG. 11C) in sorted TAMs. n=6/group.

FIG. 12. M2 macrophages have elevated lysosomal enzyme levels and activity. mRNA levels of lysosomal genes (FIG. 12) in sorted TAMs. n=6/group.

FIGS. 13A-13C. Validation of TAMs purity. FIG. 13A, Flow cytometry analysis of TAMs purified from E0771 tumors (corresponds to FIG. 14A). FIG. 13B, Quantification of other types of myeloid cell types in the purified TAM population. DC contamination was assessed by quantifying MHCIIhighCD11c+ cells, and CD11c+CD103+(Type 1 dendritic cell subset). TAN and monocyte contamination were assessed by quantifying CD11b+Ly6G+ and CD11b+Ly6Chigh cells respectively. FIG. 13C, mRNA expression levels of Zbtb46, a DC specific transcription factor, in TAMs isolated from E0771, LLC1, and B16 tumors, and bone marrow (BM)-derived M1/M2 macrophages and DCs. n=3 biological replicates/group. Statistical significance was calculated via two-tailed Student's t-test (p<0.05 values are provided); error bars indicate the mean of independent experiments±s.e.m. All measurements (n) are biological replicates.

FIGS. 14A-14D. TAMs exhibit increased lysosomal enzyme levels and activity. FIG. 14A, Isolation of mammary ATMs from tumor-free mice and TAMs from E0771 mammary tumor-bearing mice. Purity of ATMs and TAMs was validated by flow cytometry. FIG. 14B, Immunoblots of lysosomal protein levels in ATMs and TAMs. Experiment was performed once with n=3/group. FIG. 14C, DQ-OVA degradation assays of ATMs and TAMs. n=3/group. FIG. 14D, mRNA expression of lysosomal genes in TAMs isolated from E0771 tumors and thioglycolate-elicited peritoneal macrophages from tumor-free mice. n=3/group. Statistical significance was calculated via two-tailed Student's t-test (p<0.05 values are provided); error bars indicate the mean of independent experiments±s.e.m. All measurements (n) are biological replicates.

FIG. 15. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. Mammary ATMs from tumor-free mice and TAMs from E0771 mammary tumor-bearing mice (corresponds to FIG. 14C).

FIGS. 16A-16D. M2 macrophages have elevated lysosomal enzyme levels and activity. FIG. 16A, M1 and M2 HMDMs were differentiated and activated from human peripheral blood isolated monocytes. FIG. 16B-16D, mRNA levels of M1- and M2-associated genes (FIG. 16B) and lysosomal genes (FIG. 16C), and DQ-OVA degradation (FIG. 16D) in M1 and M2 HMDMs. n=4/group. All measurements (n) are biological replicates.

FIG. 17. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. M1 and M2 activated HMDMs from a healthy donor (corresponds to FIG. 16D).

FIGS. 18A-18B. M2 macrophages have elevated lysosomal enzyme levels and activity. FIG. 18A, M1-like and M2-like TAMs were sorted from human ER+breast tumors. FIG. 18B, DQ-OVA degradation assays of sorted TAMs. Patient 1: n=10 pieces/tumor 1; Patients 2-3: n=6 pieces/tumor; Patient 4: n=5 pieces/tumor. Statistical significance was calculated via two-tailed Student's t-test (p<0.05 values are provided); #, FDR<5% G-test and t-test, error bars indicate the mean of independent experiments±s.e.m. All measurements (n) are biological replicates.

FIG. 195. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. M1-like (CD206lowHLADRhigh) and M2-like (CD206highHLADRlow) TAMs from a human ER+breast cancer patient (corresponds to FIG. 18B).

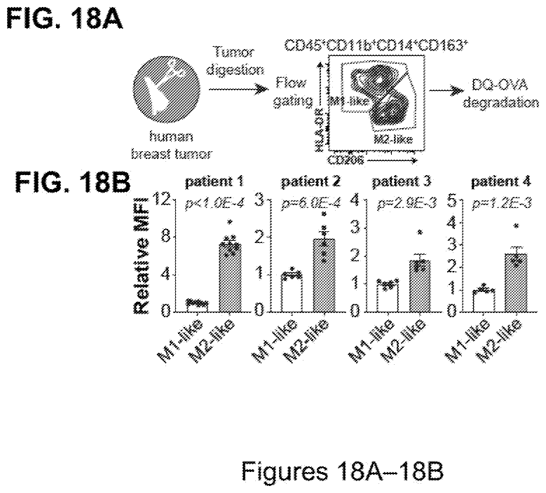

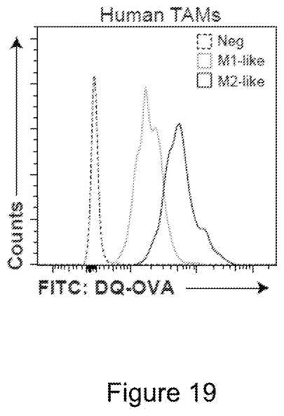

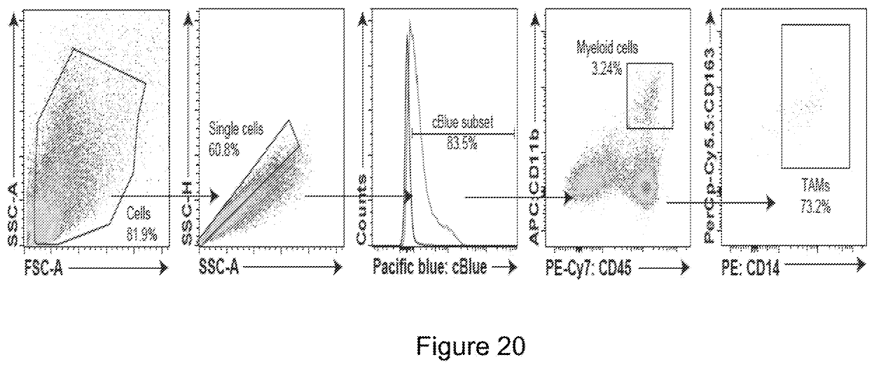

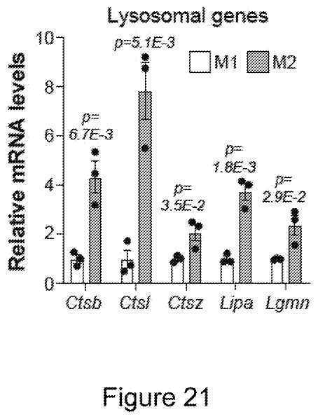

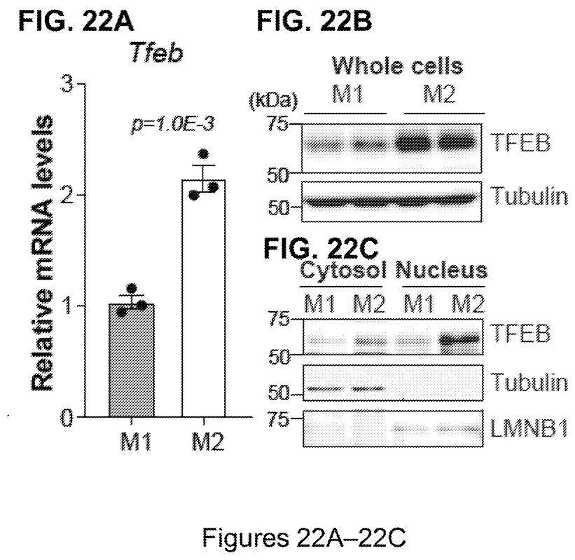

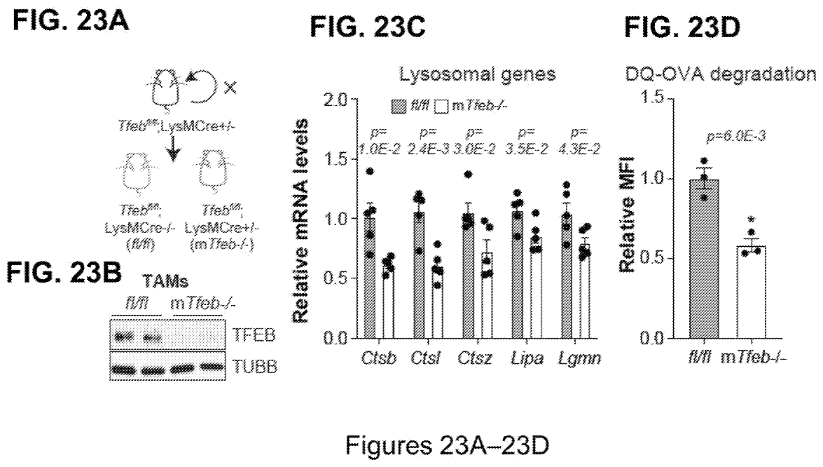

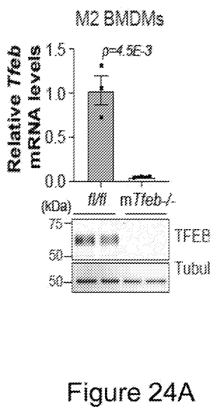

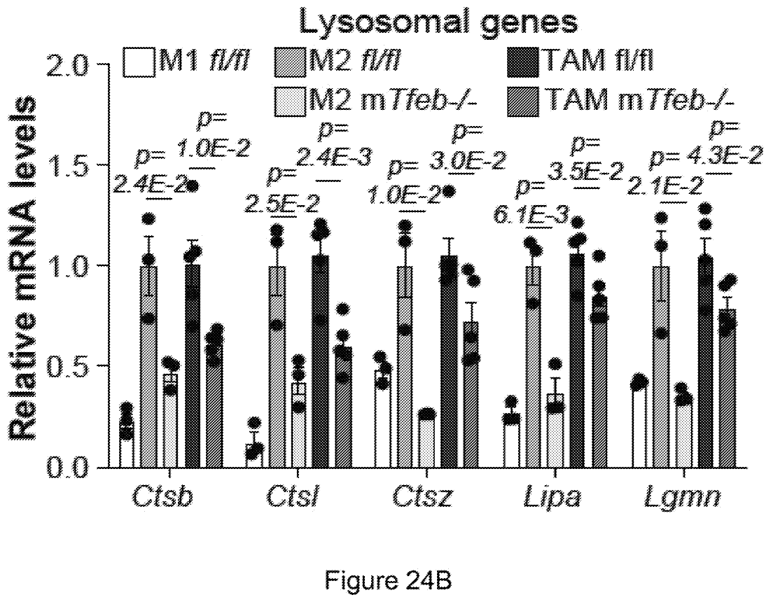

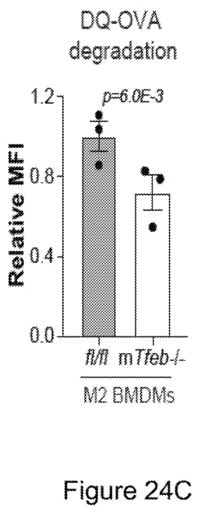

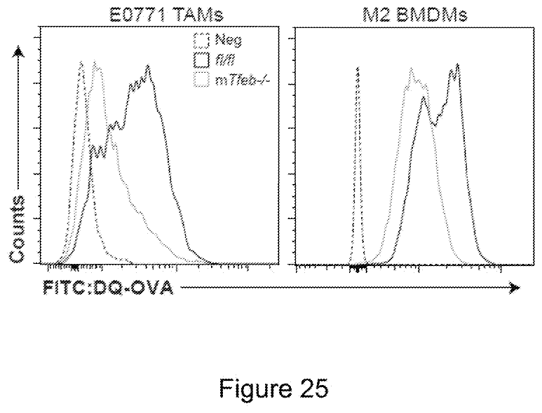

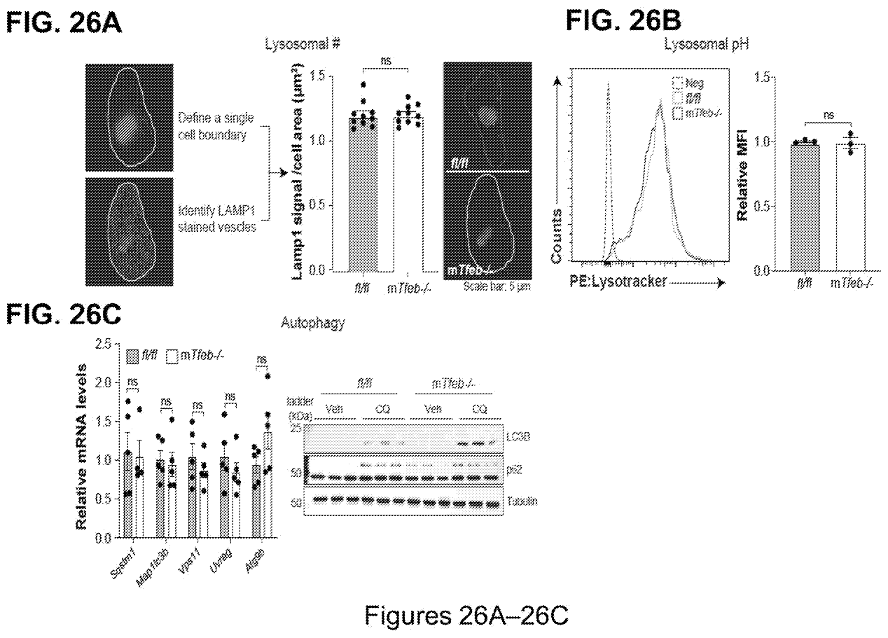

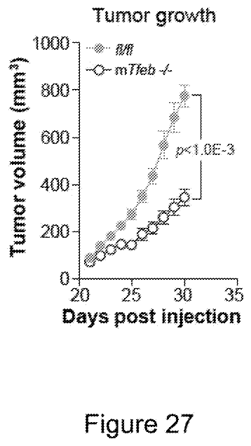

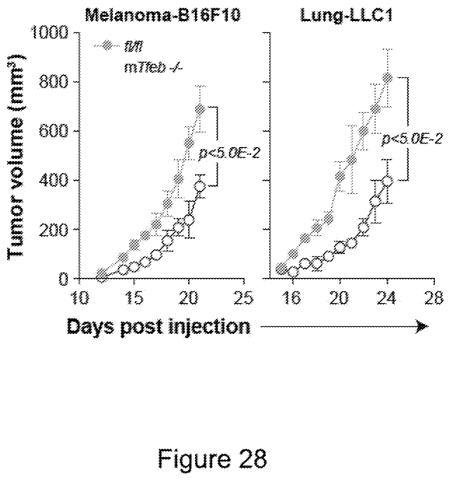

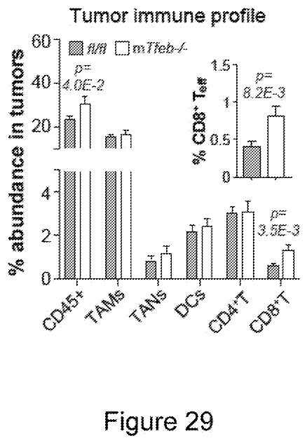

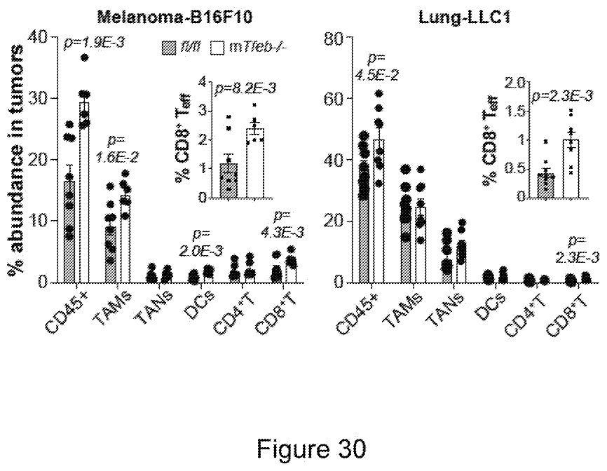

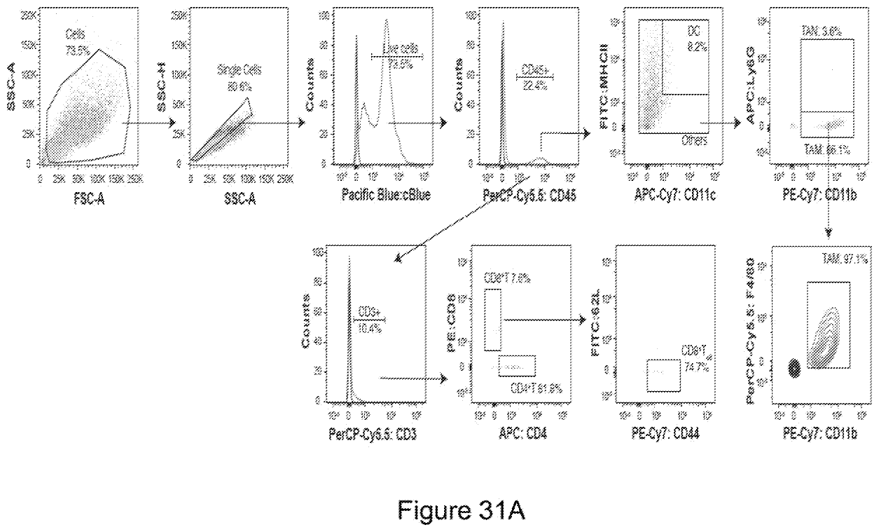

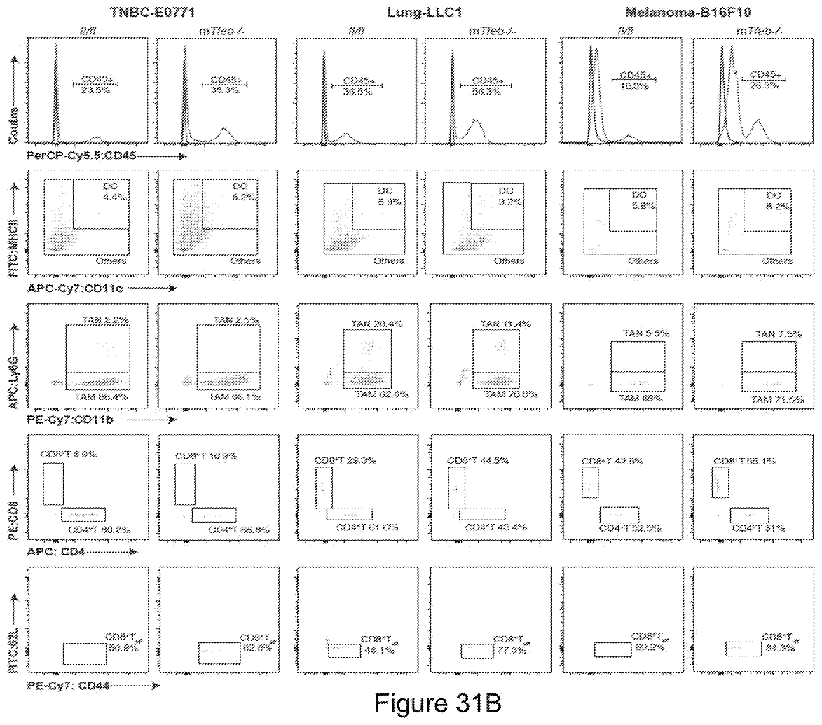

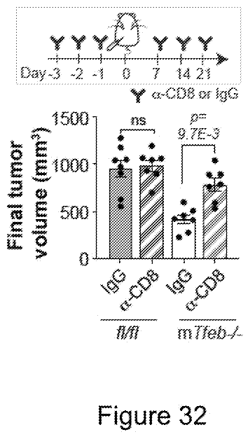

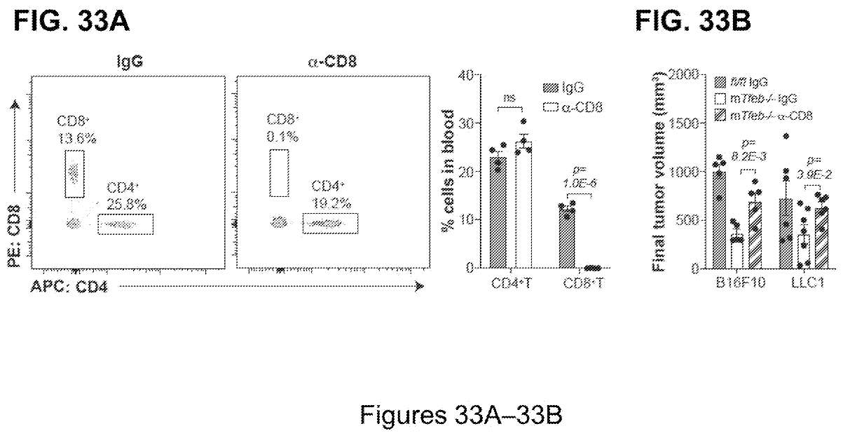

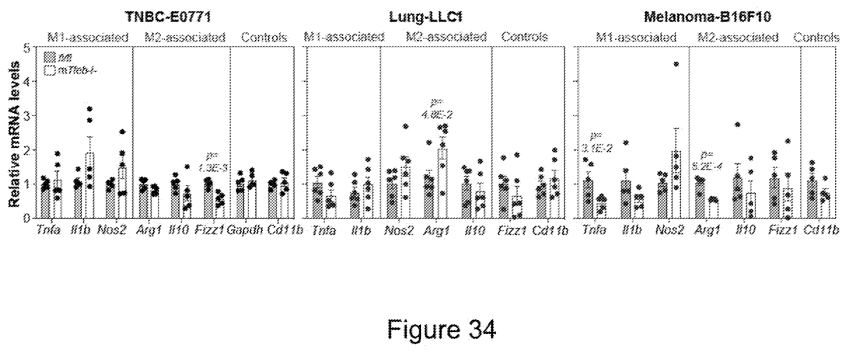

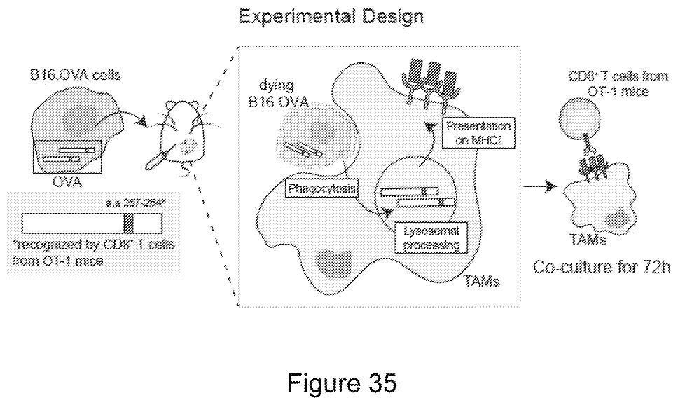

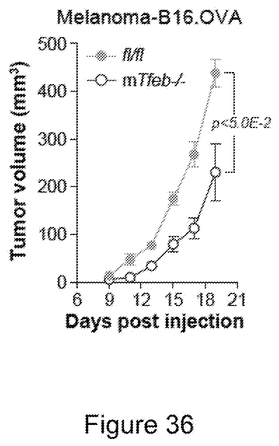

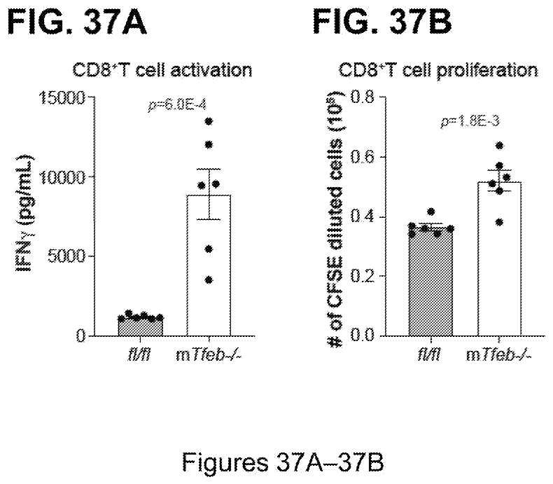

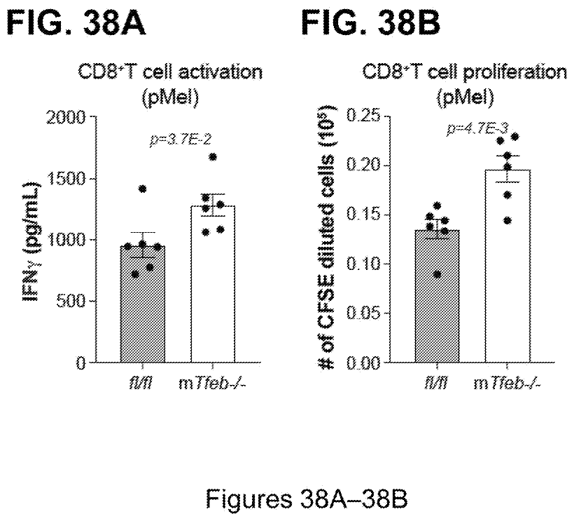

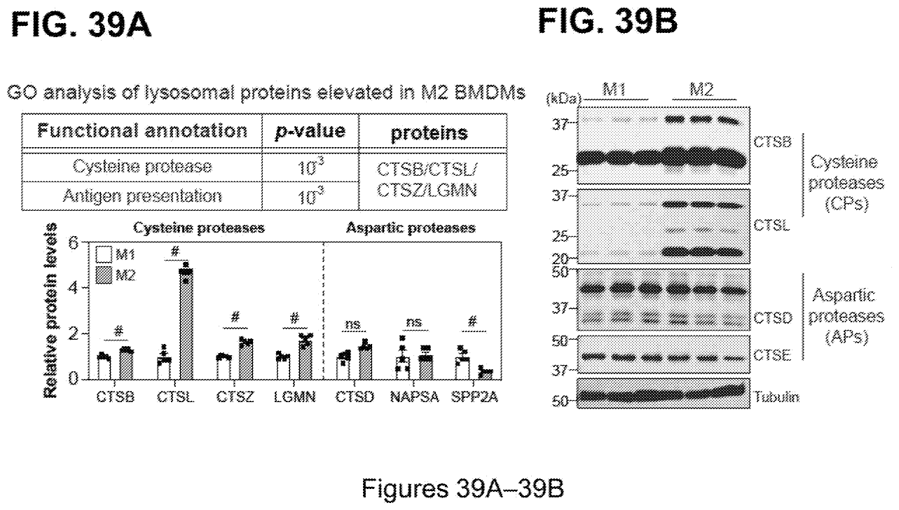

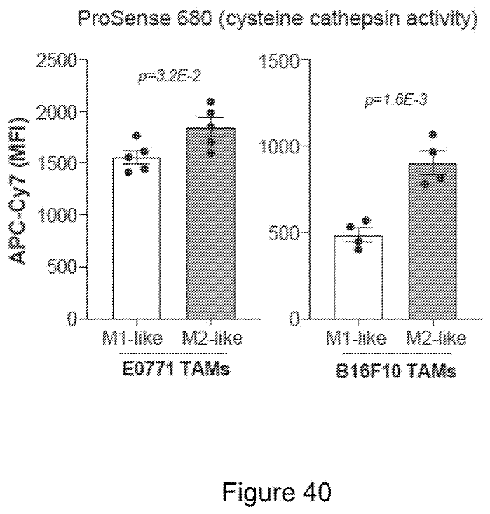

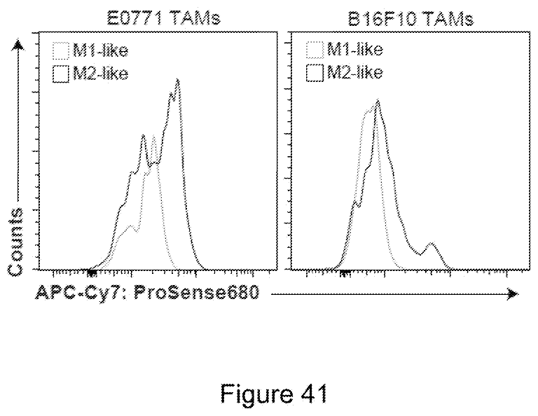

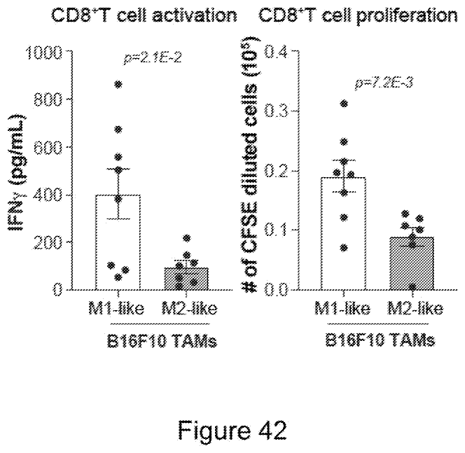

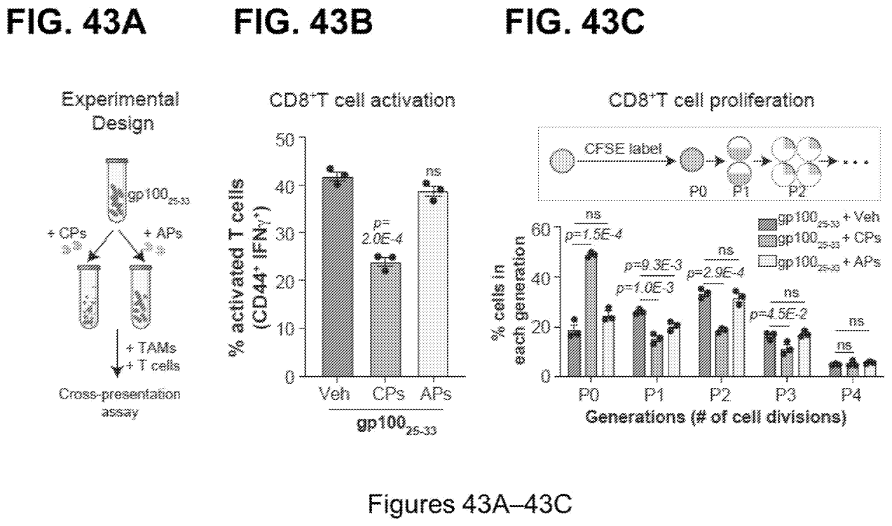

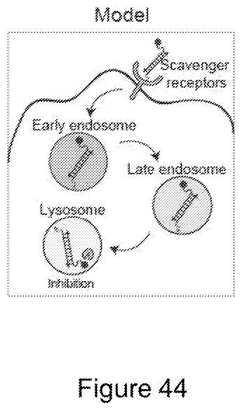

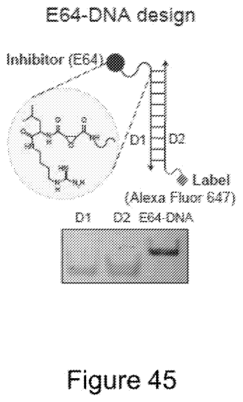

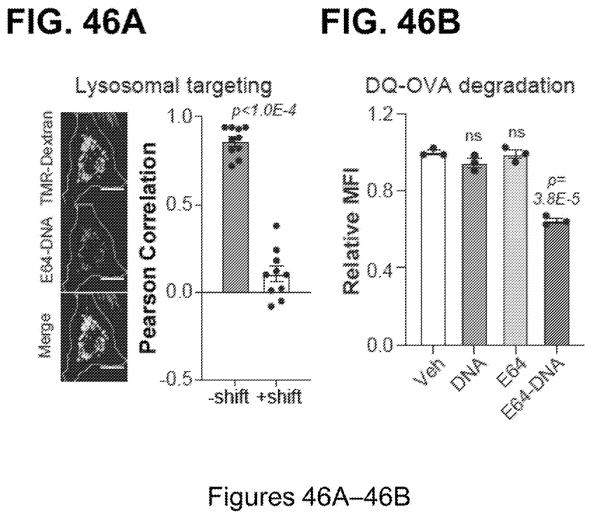

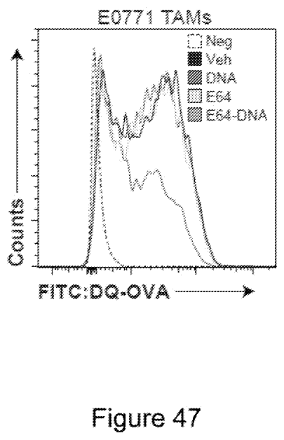

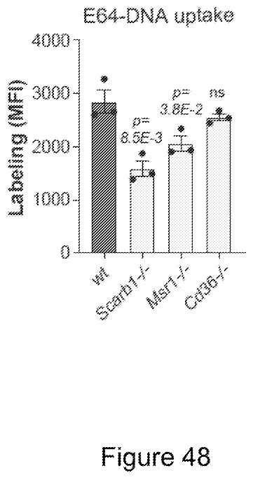

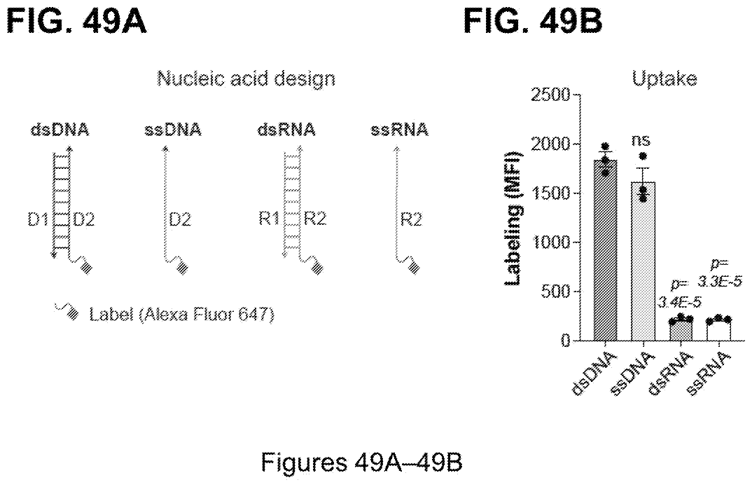

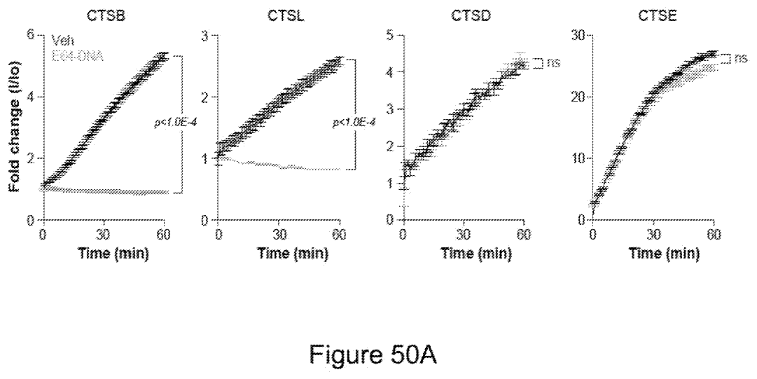

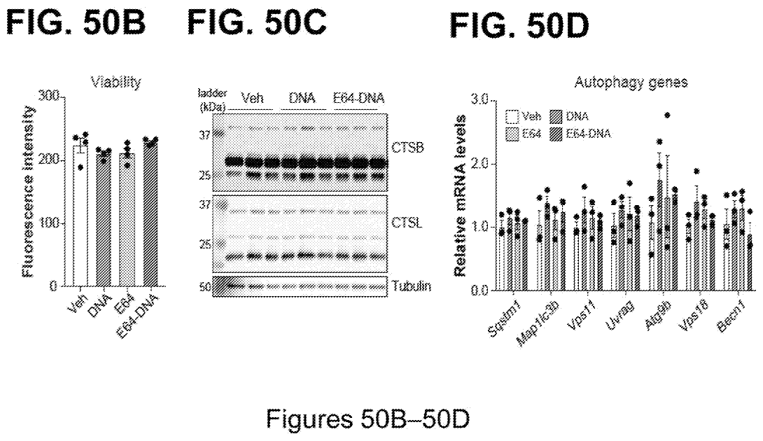

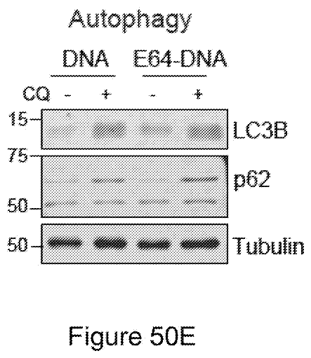

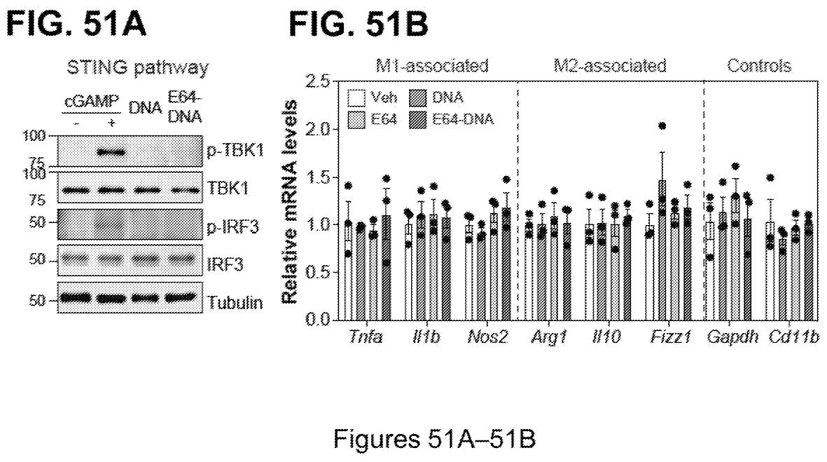

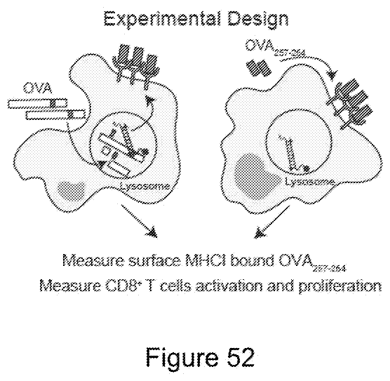

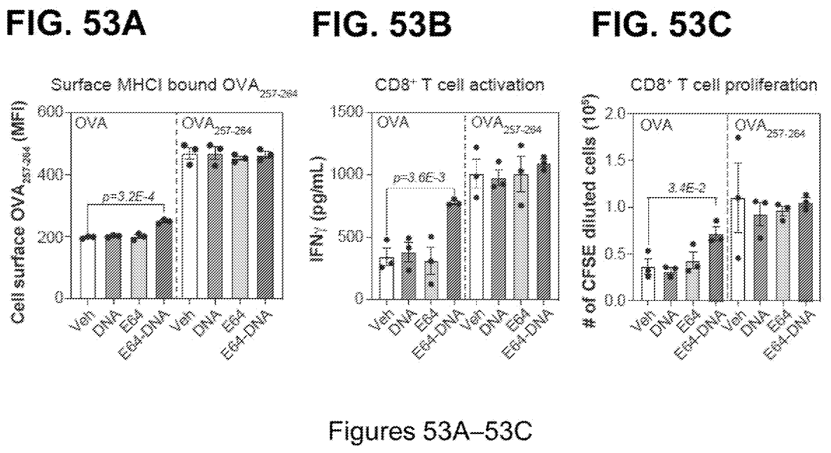

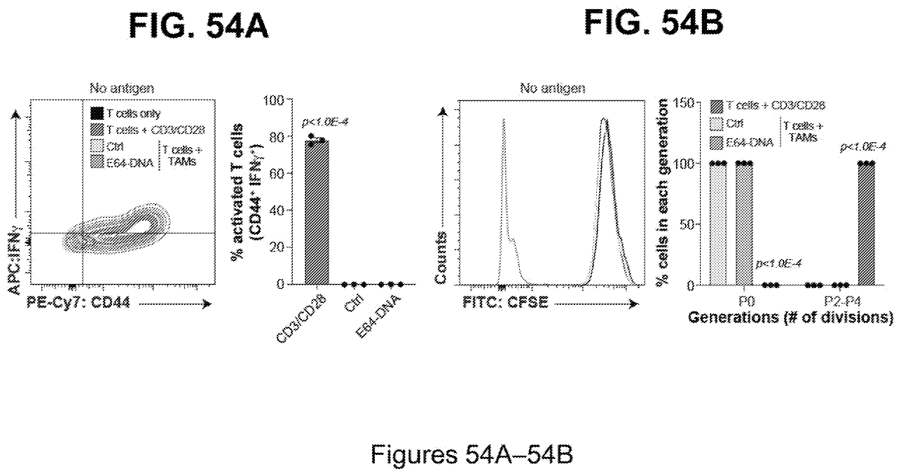

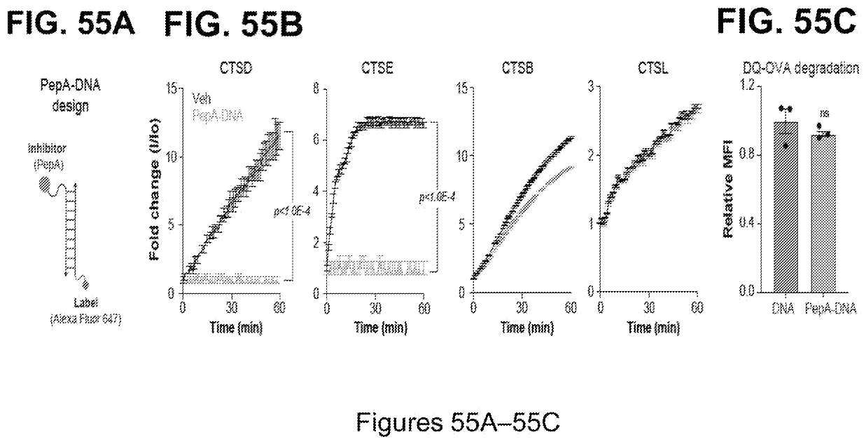

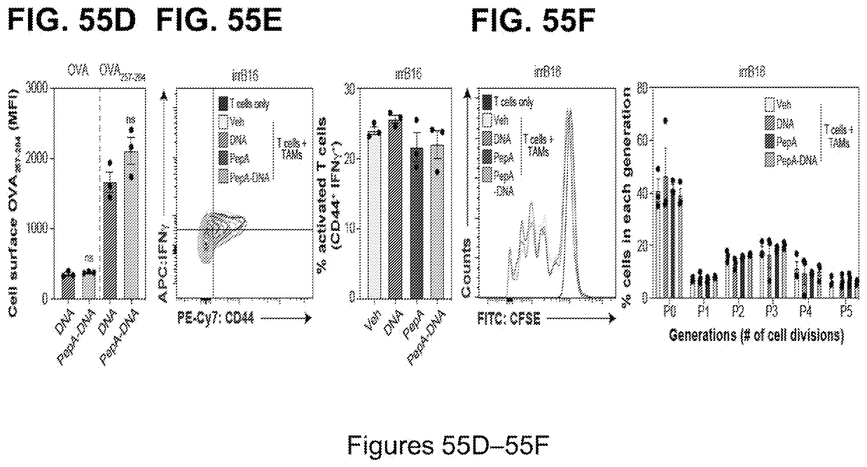

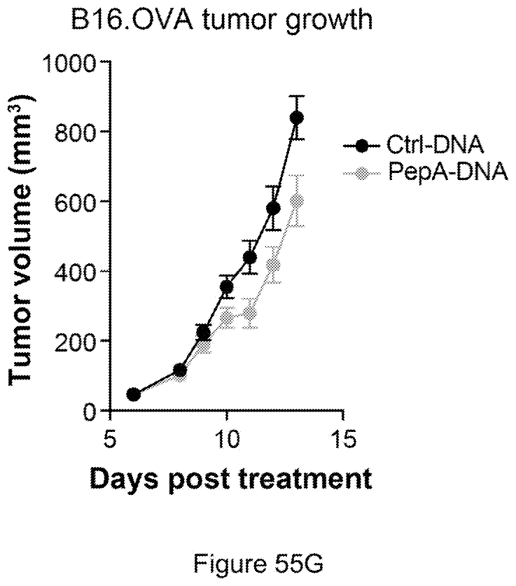

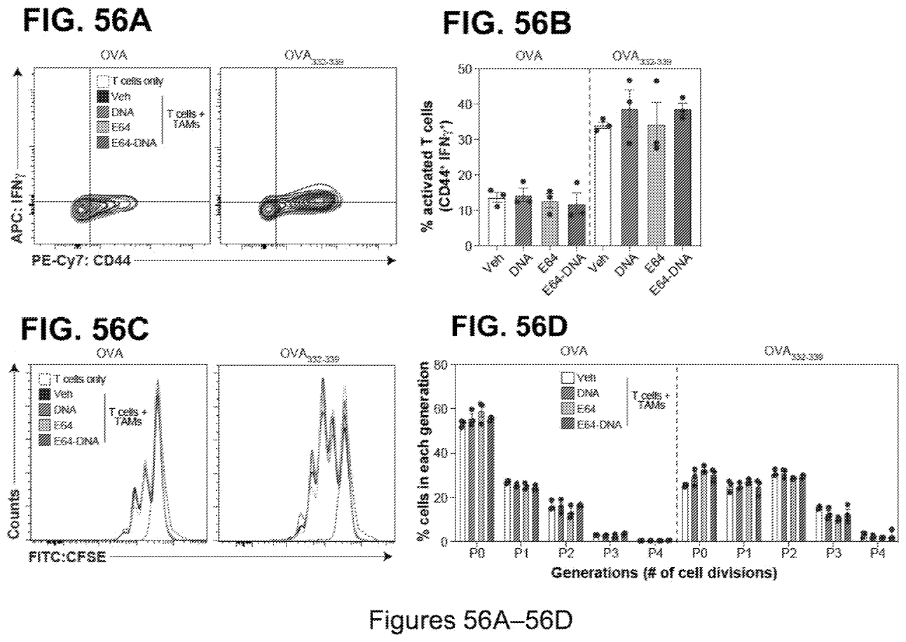

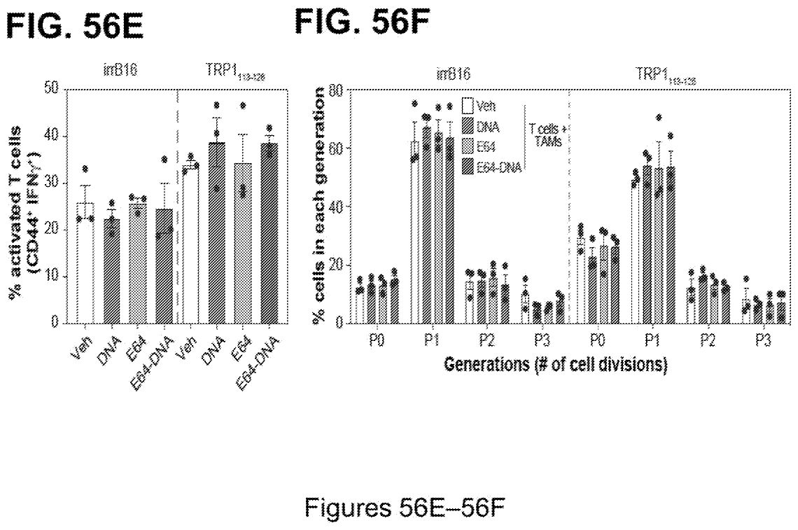

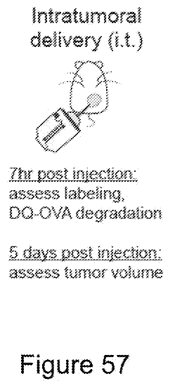

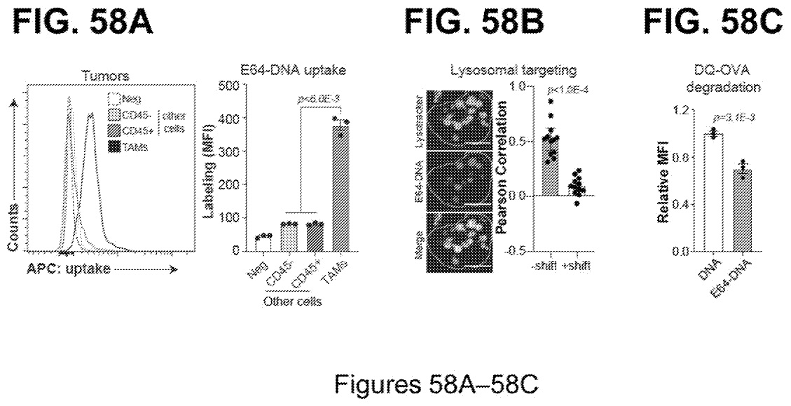

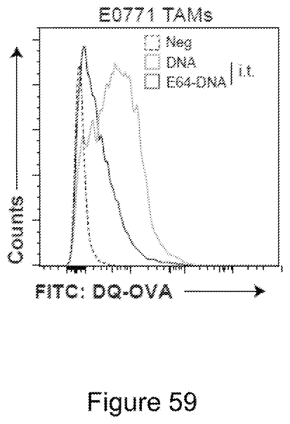

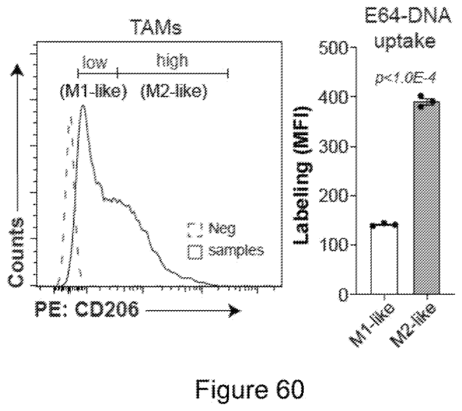

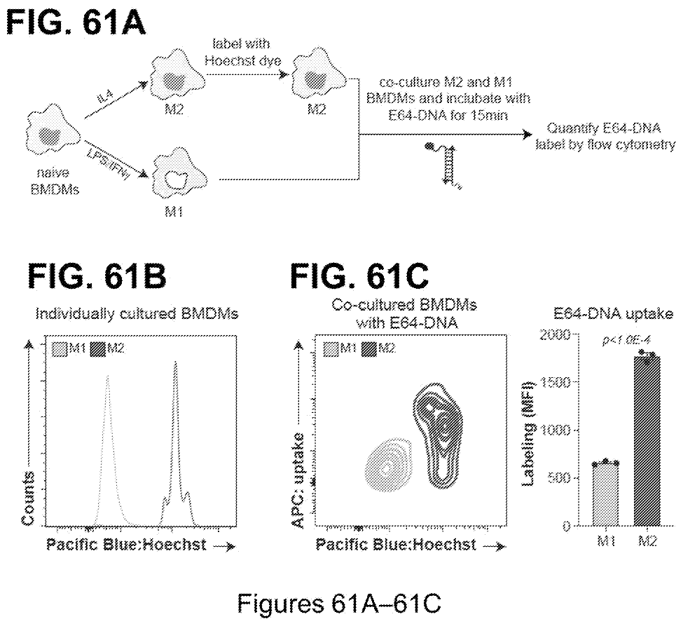

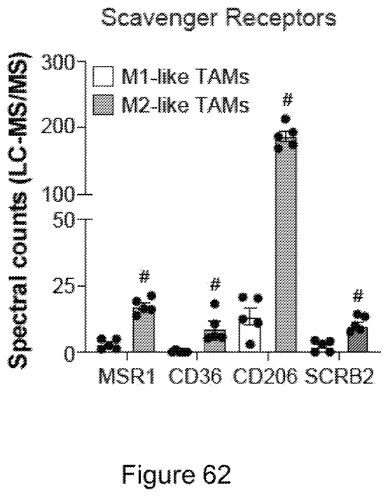

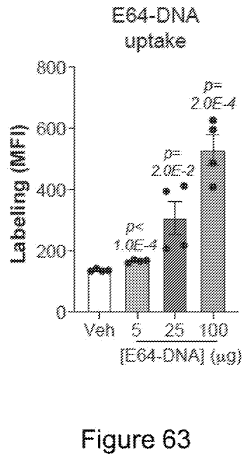

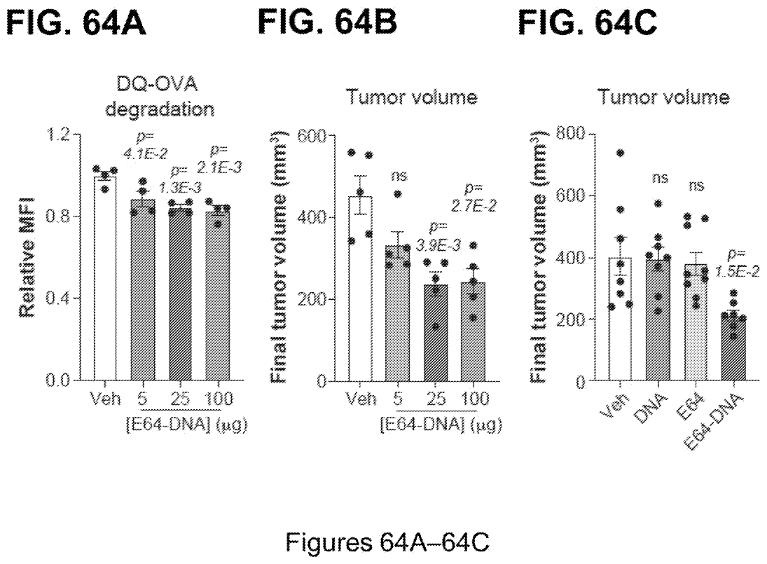

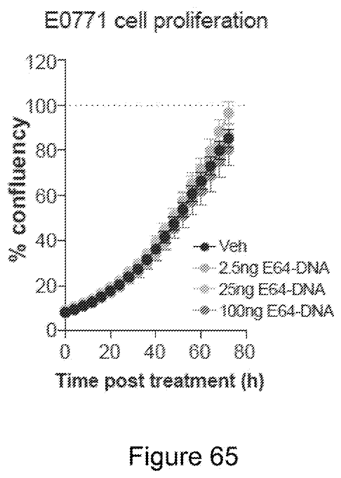

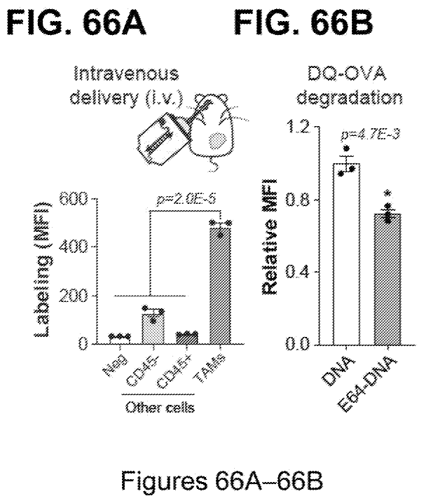

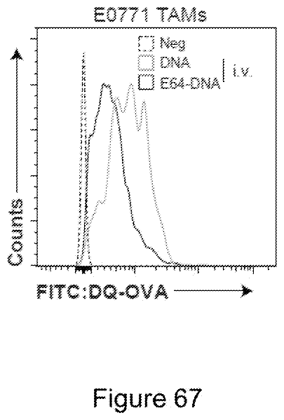

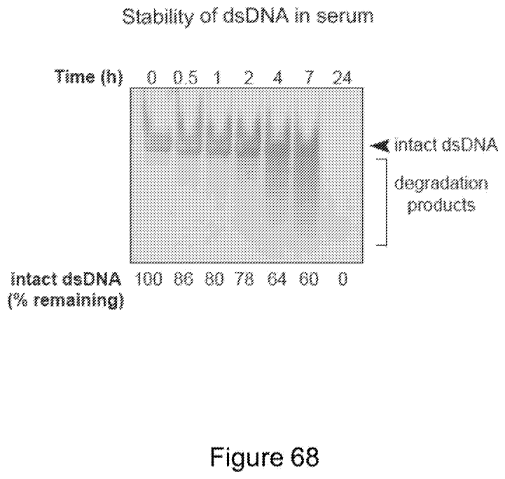

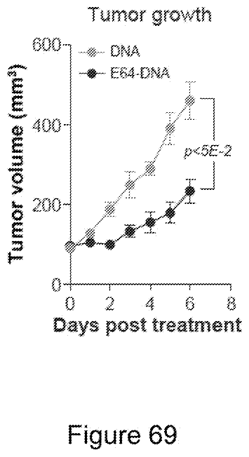

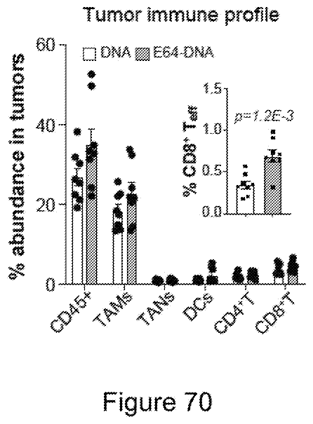

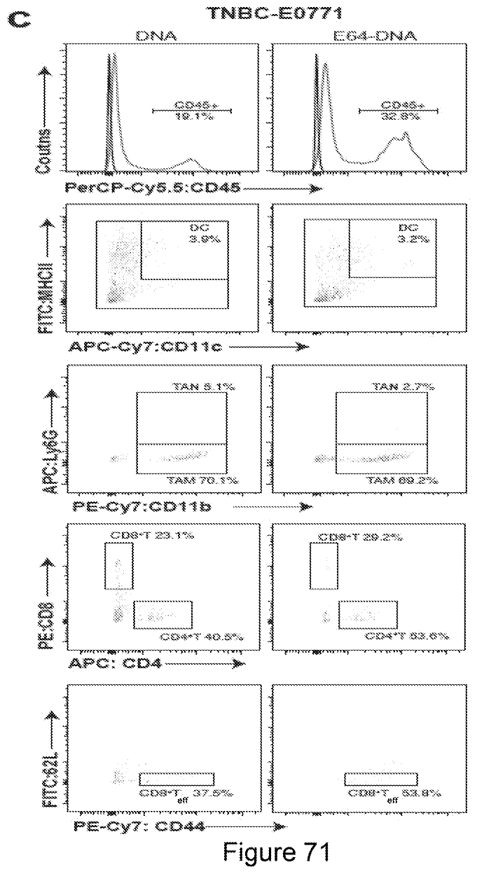

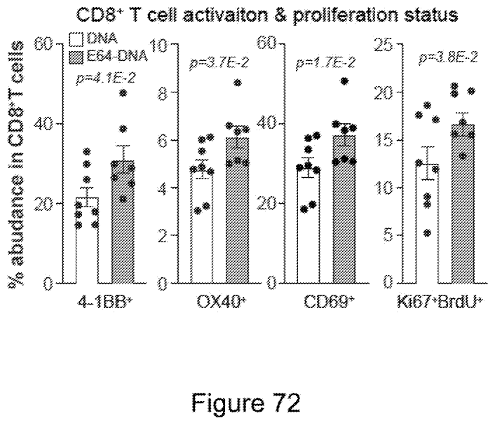

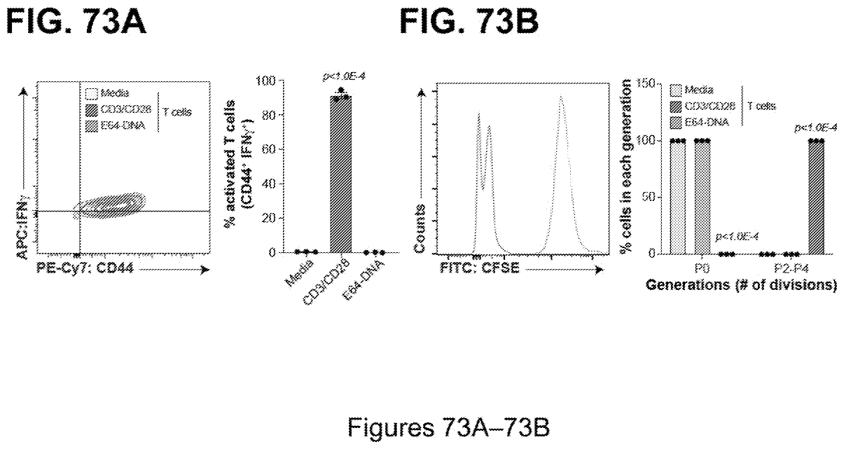

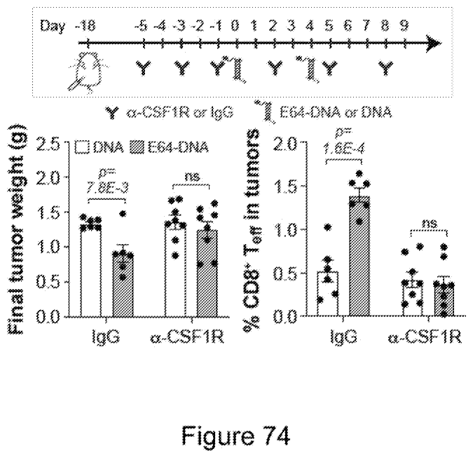

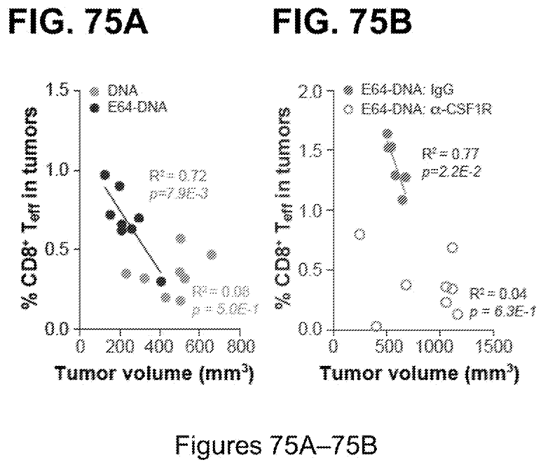

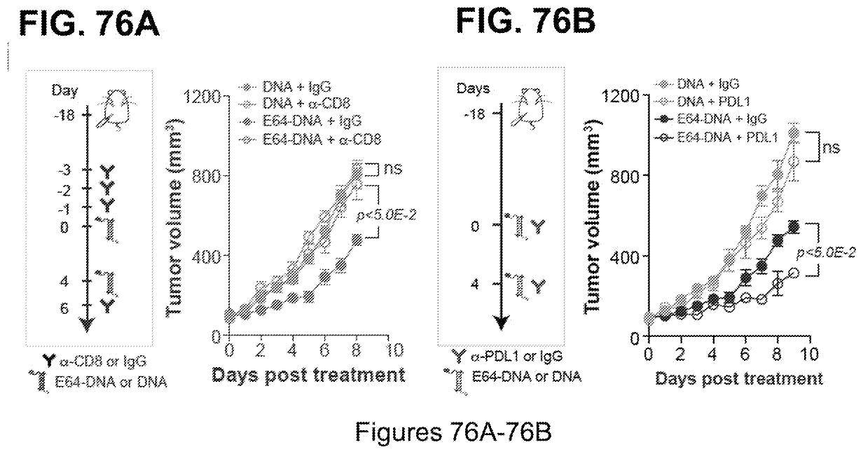

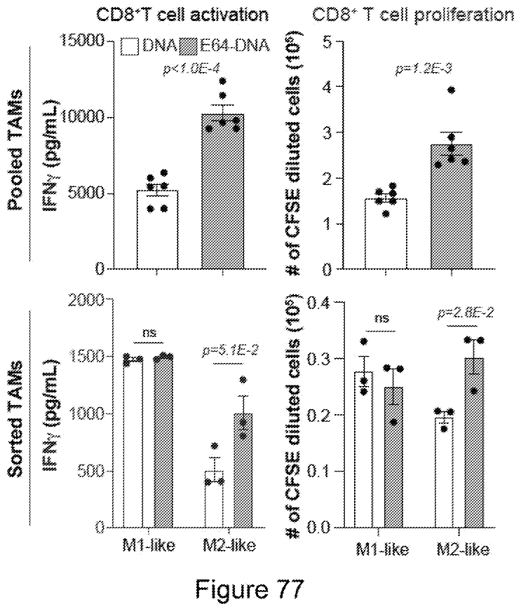

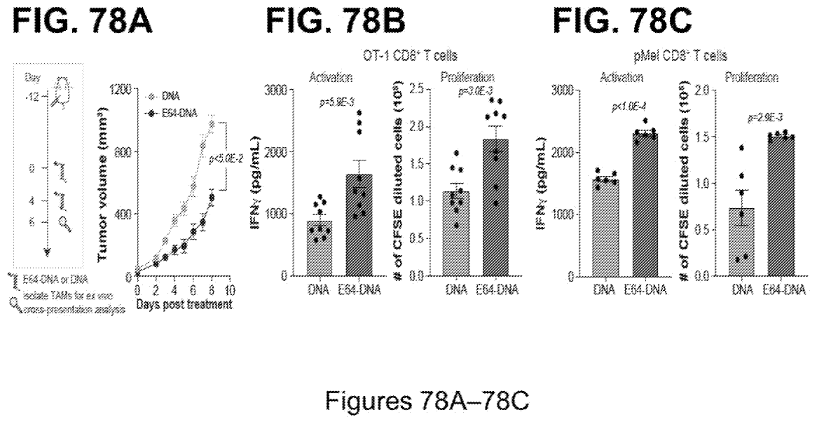

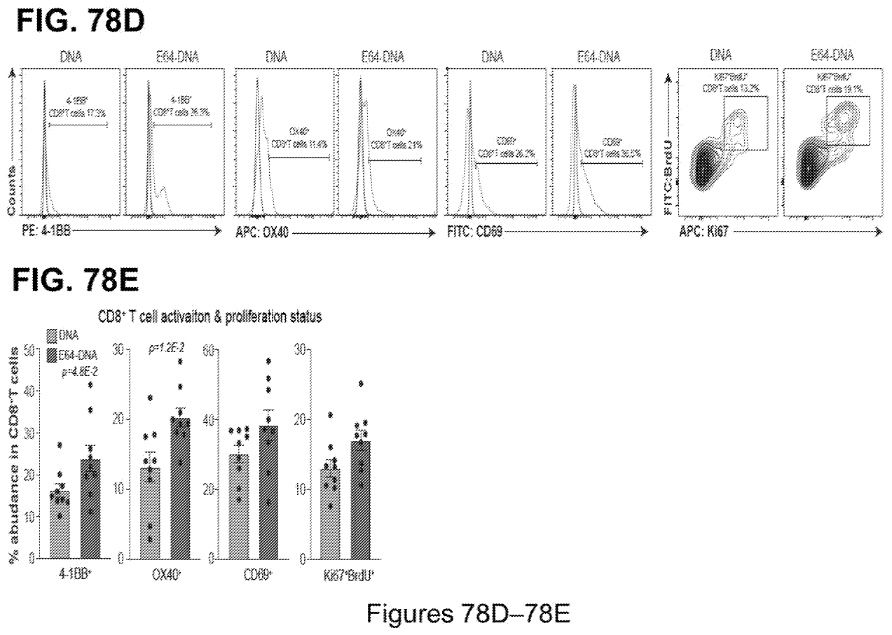

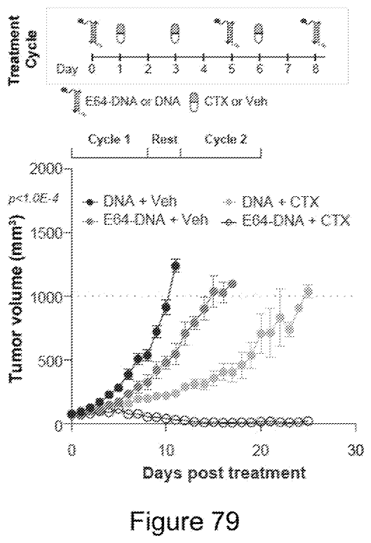

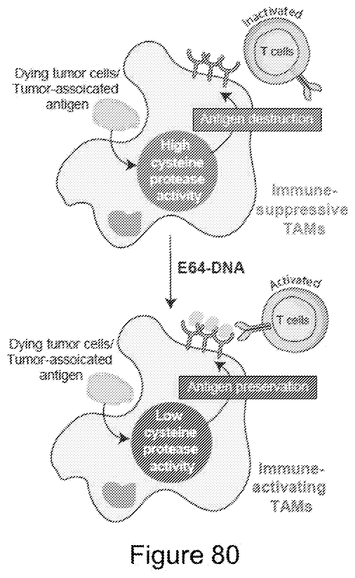

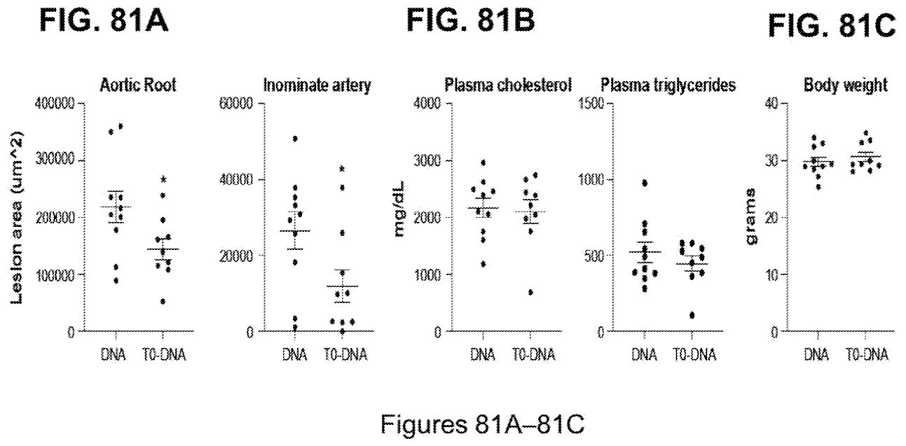

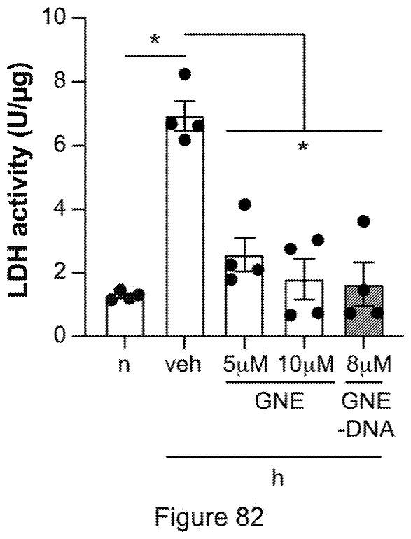

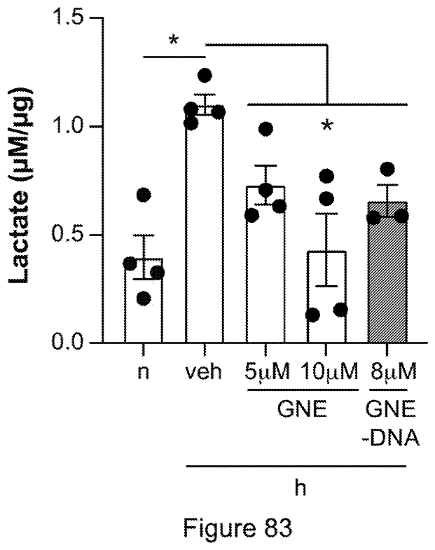

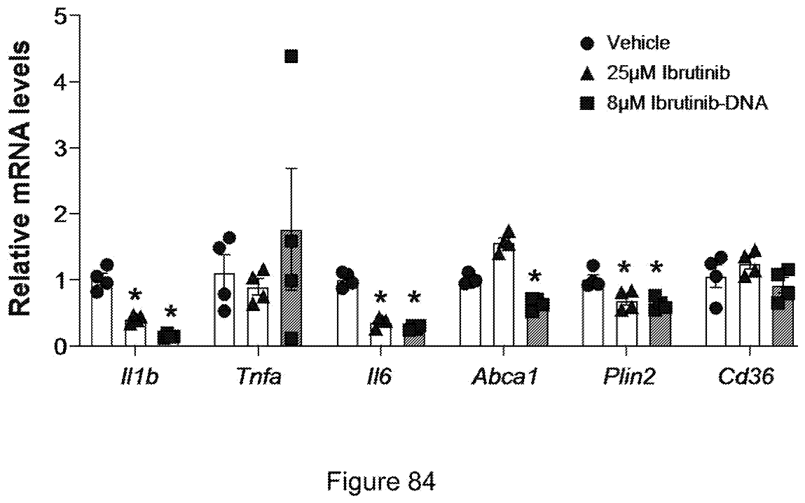

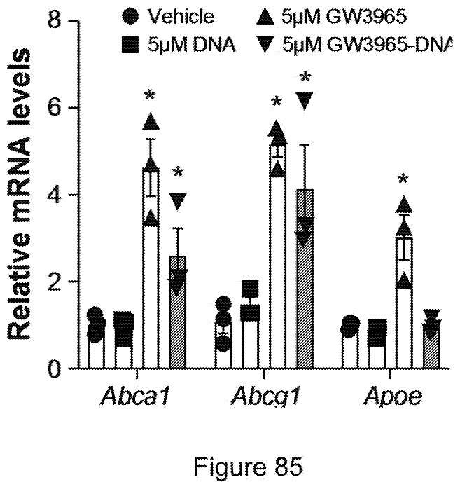

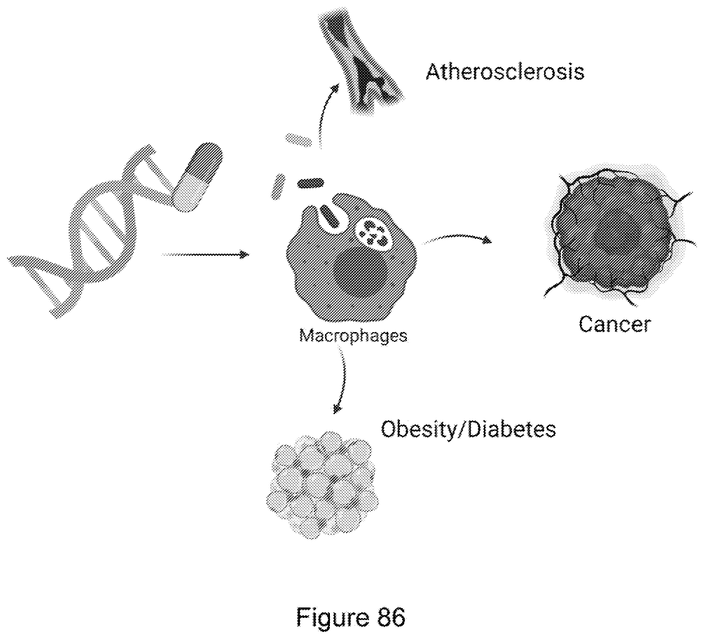

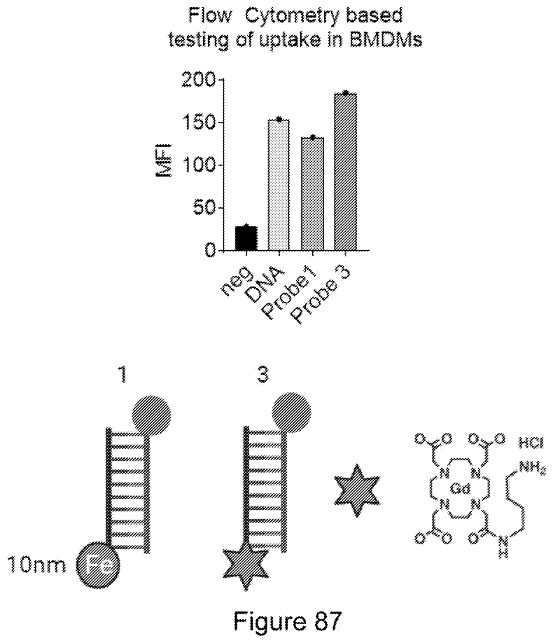

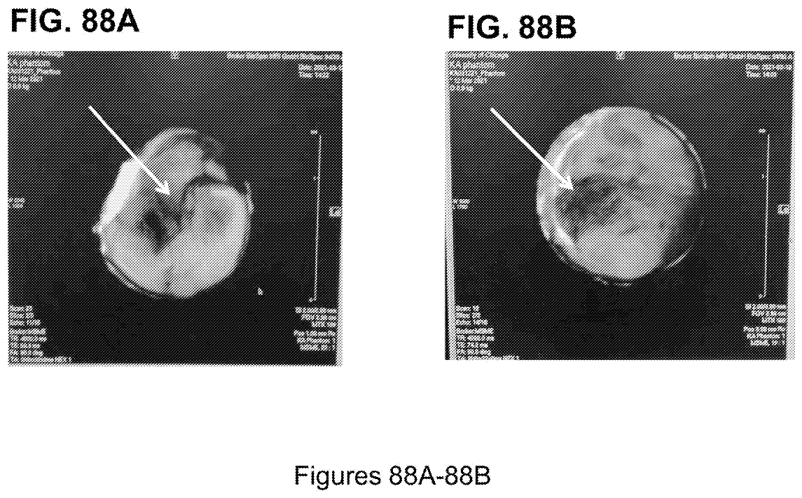

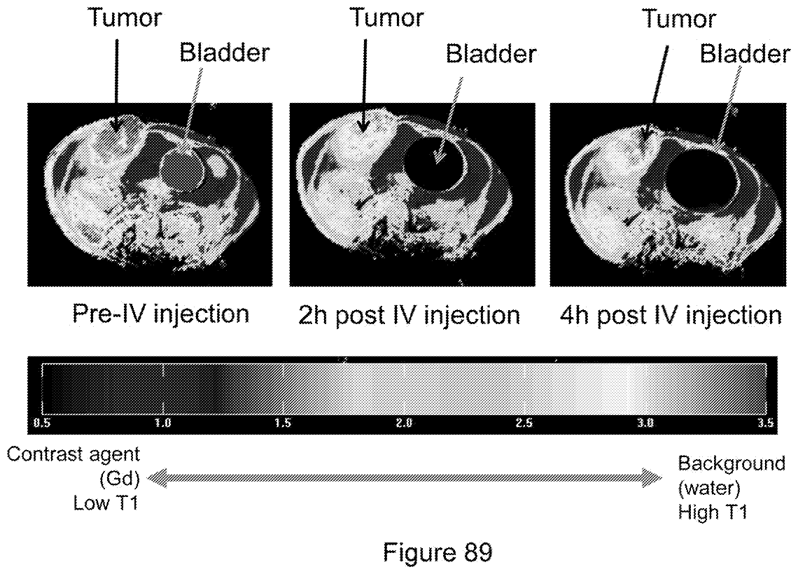

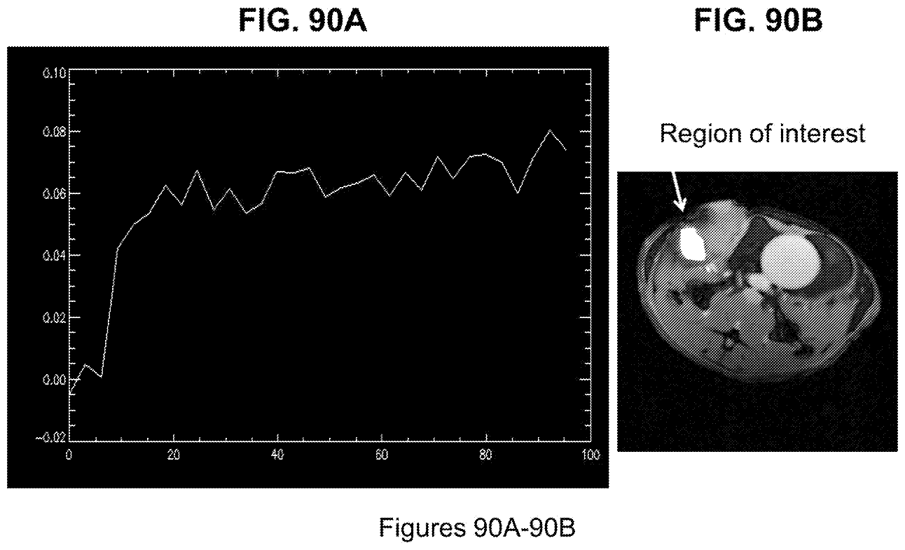

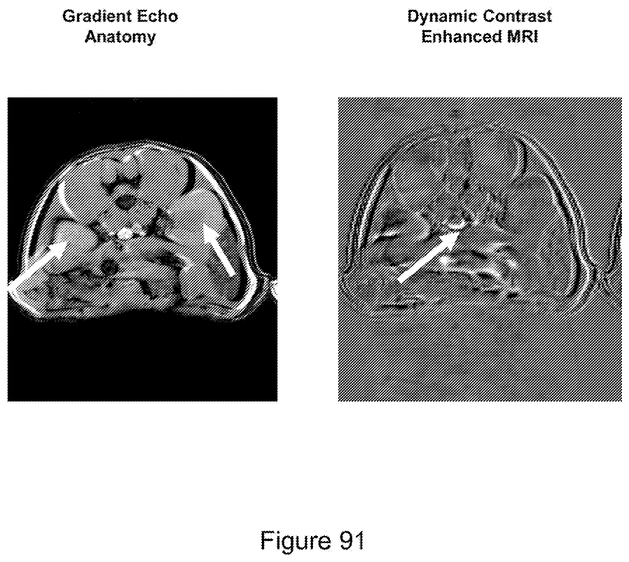

FIG. 20. Gating strategy for TAMs. Gating strategy of TAMs for analysis of M1- and M2-like TAMs from ER+breast cancer patients (corresponds to FIG. 18A).

FIG. 21. TFEB is responsible for elevated lysosomal enzymes in M2-like macrophages. mRNA levels of lysosomal genes in M1 and M2 BMDMs. n=3/group. Statistical significance was calculated via two-tailed Student's t-test (p<0.05 values are provided); error bars indicate the mean of independent experiments±s.e.m. All measurements (n) are biological replicates.

FIGS. 22A-22C. TFEB is responsible for elevated lysosomal enzymes in M2-like macrophages. FIG. 22A, Tfeb mRNA levels in M1 and M2 BMDMs. n=3/group. FIG. 22B, Immunoblot of TFEB protein levels in M1 and M2 BMDMs. Representative of 3 independent experiments. FIG. 22C, Immunoblot of cytosolic and nuclear TFEB levels in M1 and M2 BMDMs. Representative of 2 independent experiments. Statistical significance was calculated via two-tailed Student's t-test (p<0.05 values are provided); error bars indicate the mean of independent experiments±s.e.m. All measurements (n) are biological replicates.

FIGS. 23A-23D. Deleting Tfeb in myeloid cells attenuates tumor growth through CD8⁺ T cell activation. FIG. 23A, Breeding scheme of fl/fl and mTfeb−/− mice. FIGS. 23B-23D, E0771 cells were injected into the 4^{th} mammary fat pad of the right ventral side of fl/fl and mTfeb−/− mice. FIG. 23B, Immunoblot of TFEB protein levels in TAMs. Representative of three independent experiments. FIG. 23C, mRNA levels of lysosomal genes in TAMs. n=5/group. FIG. 23D, DQ-OVA degradation assays of TAMs. n=3/group. Statistical significance was calculated via two-tailed Student's t-test (p<0.05 values are provided); error bars indicate the mean of independent experiments±s.e.m. ns; not significant. CD8+Teff=effector CD8+ T cells. All measurements (n) are biological replicates.

FIGS. 24A-24C. FIG. 24A, Validation of mTfeb−/−. mRNA levels (top) n=3/group and protein levels (bottom). Representative of 3 independent experiments. FIG. 24B, A comparison of lysosomal gene expression in M1 and M2 BMDMs from fl/fl mice versus M2 BMDMs from mTfeb−/− mice, n=3/group; and a comparison of lysosomal gene expression in TAMs from fl/fl and mTfeb−/− E0771 tumors, n=4/group. FIG. 24C, DQ-OVA degradation assays of fl/fl and mTfeb−/− M2 BMDMs. n=3/group. Statistical significance was calculated via two-tailed Student's t-test (p<0.05 values are provided); error bars indicate the mean of independent experiments±s.e.m. All measurements (n) are biological replicates.

FIG. 25. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. TAMs from E0771 tumors (left) and M2 BMDMs (right) from fl/fl and mTfeb−/− mice (corresponds to FIG. 23D and FIG. 24C respectively).

FIGS. 26A-26C. TAMs from mTfeb−/− mice exhibit improved antigen cross-presentation with minimal phenotypic changes. TAMs were isolated from E0771 tumors. FIG. 26A, Quantification of lysosomes in fl/fl and mTfeb−/− TAMs based on LAMP1 immunostaining. Schematic for quantification (left). Quantification of average LAMP1 signal/cell area (n=10/group) with an average of >40 cells/field (middle). Representative images (right). LAMP1 (red) and DAPI (blue). FIG. 26B, Quantification of lysosomal pH in fl/fl and mTfeb−/− TAMs based on lysotracker staining. Representative flow cytometry image (left). Quantification of relative MFI of lysotracker signal (right). n=3/group. FIG. 26C, Autophagy gene expression in fl/fl and mTfeb−/− TAMs (left, n=5). LC3B and p62 protein levels in fl/fl and mTfeb−/− TAMs following treatment with vehicle (Veh) or chloroquine (CQ, 50 μM) for 24h (right). Veh=H2O. Experiment was performed once with n=3/group. d, M1- and M2-associated gene expression in TAMs from fl/fl and mTfeb−/− E0771 tumors (left, n=5/group), LLC1 tumors (middle, n=5/group) and B16F10 tumors (right, n=4 group). Statistical significance was calculated via two-tailed Student's t-test. ns; not significant. All measurements (n) are biological replicates.

FIG. 27. Deleting Tfeb in myeloid cells attenuates tumor growth through CD8+ T cell activation. E0771 tumor growth. fl/fl: n=12/group, mTfeb−/−: n=11/group. Statistical significance was calculated via two-tailed Student's t-test (p<0.05 values are provided); error bars indicate the mean of independent experiments±s.e.m. ns; not significant. CD8⁺ T_{eff} effector CD8⁺ T cells. All measurements (n) are biological replicates.

FIG. 28. Deleting Tfeb in myeloid cells attenuates tumor growth via CD8⁺ T cells (B16F10 & LLC1 models). B16F10 tumor growth rates in fl/fl (n=14) and mTfeb−/− (n=10) mice (left). LLC1 tumor growth rates in fl/fl (n=10) and mTfeb−/− (n=8) mice (right). Statistical significance was calculated via two-tailed Student's t-test (p<0.05 values are provided); error bars indicate the mean of independent experiments±s.e.m. ns; not significant. All measurements (n) are biological replicates.

FIG. 29. Deleting Tfeb in myeloid cells attenuates tumor growth through CD8+ T cell activation. Tumor immune cell composition. fl/fl: n=10/group, mTfeb−/−: n=11/group. Statistical significance was calculated via two-tailed Student's t-test (p<0.05 values are provided); error bars indicate the mean of independent experiments±s.e.m. ns; not significant. CD8+Teff=effector CD8+ T cells. All measurements (n) are biological replicates.

FIG. 30. Deleting Tfeb in myeloid cells attenuates tumor growth via CD8⁺ T cells (B16F10 & LLC1 models). Tumor immune cell composition in B16F10 tumor bearing fl/fl (n=8) and mTfeb−/− (n=6) mice; Tumor immune cell composition in LLC1 tumor bearing fl/fl (n=9) and mTfeb−/− (n=8) mice. CD8+Teff=effector CD8+ T cells. Statistical significance was calculated via two-tailed Student's t-test (p<0.05 values are provided); error bars indicate the mean of independent experiments±s.e.m. ns; not significant. All measurements (n) are biological replicates.

FIGS. 31A-31B. Gating strategy and representative flow cytometry data for tumor immune cell composition. FIG. 31A, Gating strategy for flow cytometric analyses of tumor immune cell composition. FIG. 31B, Representative flow cytometry data for immune cell composition in E0771 (left), LLC1 (middle), and B16F10 (right) tumors from fl/fl and mTfeb−/− mice.

FIG. 32. Deleting Tfeb in myeloid cells attenuates tumor growth through CD8⁺ T cell activation. Final tumor volumes in mice treated with IgG or α-CD8 antibodies. Experimental design (top). Final tumor volume (bottom). fl/fl: n=7group, mTfeb−/−: n=8/group. Statistical significance was calculated via two-tailed Student's t-test (p<0.05 values are provided); error bars indicate the mean of independent experiments±s.e.m. ns; not significant. CD8+Teff=effector CD8+ T cells. All measurements (n) are biological replicates.

FIGS. 33A-33B. Deleting Tfeb in myeloid cells attenuates tumor growth via CD8⁺ T cells (B16F10 & LLC1 models). FIG. 33A, Blood CD8⁺ T cell levels in mice treated with α-CD8 or IgG antibodies. Representative flow cytometry data (left). Quantification of CD8⁺ and CD4⁺ T cells (right). n=4/group. FIG. 33B, Final tumor volume in B16F10 (n=5/group) and LLC1 (fl/fl: n=6, mTfeb−/−: n=7 (IgG), n=6 (α-CD8)) tumor bearing fl/fl and mTfeb−/− mice treated with IgG or α-CD8 antibodies. Statistical significance was calculated via two-tailed Student's t-test (p<0.05 values are provided); error bars indicate the mean of independent experiments±s.e.m. ns; not significant. All measurements (n) are biological replicates.

FIG. 34. TAMs from mTfeb−/− mice exhibit improved antigen cross-presentation with minimal phenotypic changes. M1- and M2-associated gene expression in TAMs from fl/fl and mTfeb−/− E0771 tumors (left, n=5/group), LLC1 tumors (middle, n=5/group) and B16F10 tumors (right, n=4 group). Statistical significance was calculated via two-tailed Student's t-test (p<0.05 values are provided); error bars indicate the mean of independent experiments±s.e.m. ns; not significant. All measurements (n) are biological replicates.

FIG. 35. Experimental design for antigen cross-presentation using the B16.OVA-OT-1 model.

FIG. 36. Deleting Tfeb in myeloid cells attenuates tumor growth through CD8⁺ T cell activation. B16.0VA tumor growth in fl/fl and mTfeb−/− mice. n=7/group. Statistical significance was calculated via two-tailed Student's t-test (p<0.05 values are provided); error bars indicate the mean of independent experiments±s.e.m. ns; not significant. CD8⁺ T_{eff}=effector CD8⁺ T cells. All measurements (n) are biological replicates.

FIG. 37A-37B. Deleting Tfeb in myeloid cells attenuates tumor growth through CD8⁺ T cell activation. OT-1-CD8⁺ T-cell activation (FIG. 37A) and proliferation (FIG. 37B) following co-culture with TAMs isolated from fl/fl and mTfeb−/− B16.0VA tumors. n=6/group Statistical significance was calculated via two-tailed Student's t-test (p<0.05 values are provided); error bars indicate the mean of independent experiments±s.e.m. ns; not significant. CD8⁺ T_{eff}=effector CD8⁺ T cells. All measurements (n) are biological replicates.

FIG. 384A-38B. TAMs from mTfeb−/− mice exhibit improved antigen cross-presentation with minimal phenotypic changes. Quantification of pMel-CD8+ T cell activation (e) and proliferation (f) following co-culture with TAMs isolated from fl/fl and mTfeb−/− B16.0VA tumors. n=6/group. Statistical significance was calculated via two-tailed Student's t-test (p<0.05 values are provided); error bars indicate the mean of independent experiments±s.e.m. ns; not significant. All measurements (n) are biological replicates.

FIGS. 39A-39B. Lysosomal cysteine proteases are elevated in M2 macrophages. FIG. 39A, Top two pathways from GO analysis of up-regulated lysosomal proteins in M2 BMDMs (top, p<0.05, Fisher's exact test with Benjamini-Hochberg correction). Cysteine protease and aspartic protease levels in M1/M2 BMDMs quantified by spectral counting (bottom, n=5/group). FIG. 39B, Immunoblots of representative cysteine and aspartic protease in M1 and M2 BMDMs. Representative of at least 2 independent experiments. Statistical significance was calculated via two-tailed Student's t-test. ns; not significant. All measurements (n) are biological replicates.

FIG. 40. Lysosomal cysteine proteases are elevated in M2 macrophages. Cysteine cathepsin activity of M1-like and M2-like TAMs from E0771 (n=5/group) or B16F10 (n=4/group) tumors measured with the ProSense 680 fluorescent imaging agent. Statistical significance was calculated via two-tailed Student's t-test (p<0.05 values are provided); error bars indicate the mean of independent experiments±s.e.m. ns; not significant. All measurements (n) are biological replicates.

FIG. 41. Representative flow cytometry analyses of DQ-OVA degradation and cysteine protease activity (ProSense 680). Representative flow cytometry data on cysteine protease activity (measured by ProSense 680 fluorescence imaging agent) in M1-like and M2-like TAMs sorted from E0771 and B16F10 tumors (corresponds to FIG. 40).

FIG. 42. Lysosomal cysteine proteases are elevated in M2 macrophages. pMel-CD8⁺ T-cell activation (left) and proliferation (right) following co-culture with M1-like and M2-like sorted TAMs isolated B16F10 tumors. n=7-8/group. Statistical significance was calculated via two-tailed Student's t-test (p<0.05 values are provided); error bars indicate the mean of independent experiments±s.e.m. ns; not significant. All measurements (n) are biological replicates.

FIGS. 43A-43C. Lysosomal cysteine proteases are elevated in M2 macrophages. FIG. 43A, Experimental design for in vitro antigen destruction by aspartic or cysteine proteases. FIG. 43B-43C, pMel-CD8⁺ T cell activation (FIG. 43B) and proliferation (FIG. 43C) after 72h of co-culture with TAMs pre-stimulated with diluted gp10025-33 digestion solution. n=3/group. #, FDR<5% G-test and t-test (from shotgun proteomics analyses); Statistical significance was calculated via two-tailed Student's t-test (p<0.05 values are provided); error bars indicate the mean of independent experiments±s.e.m. ns; not significant. All measurements (n) are biological replicates.

FIG. 44. Scheme of E64-DNA trafficking to lysosome.

FIG. 45. E64-DNA design. One strand (D1) is conjugated with E64 on its 5′ end and the other (D2) with Alexa Fluor 647 (top). E64-DNA purity and integrity was validated by native polyacrylamide gel electrophoresis (bottom). Representative of at least 3 independent experiments.

FIGS. 46A-46B. A lysosome-targeted DNA nanodevice (E64-DNA) promotes antigen cross-presentation by TAMs. FIG. 43A, Representative images (left) and Pearson correlation (right) of co-localization of TMR-Dextran labeled lysosomes (green) with E64-DNA (red). Pearson correlation with and without a 20-pixel shift (˜lysosome diameter) of the green signal. n=15 cells/group. scale bar=1011.m. FIG. 43B, DQ-OVA degradation by TAMs treated with E64-DNA, DNA, or E64 (100 nM) for 2h. n=3/group. Statistical significance was calculated via two-tailed Student's t-test (p<0.05 values are provided); error bars indicate the mean of independent experiments±s.e.m. ns; not significant. TAMs were isolated from E0771 tumors. All measurements (n) are biological replicates.

FIG. 47. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. TAMs from E0771 tumors treated with E64-DNA, DNA, or E64 (100 nM), or vehicle (Veh; phosphate-buffered saline) for 2h ex vivo (corresponds to FIG. 43B).

FIG. 48. A lysosome-targeted DNA nanodevice (E64-DNA) promotes antigen cross-presentation by TAMs. E64-DNA uptake by M2 BMDMs from wt, Scarb1−/−, Msr1−/−, or Cd36−/− mice. Uptake was quantified by flow cytometry; n=3/group. Statistical significance was calculated via two-tailed Student's t-test (p<0.05 values are provided); error bars indicate the mean of independent experiments±s.e.m. ns; not significant. TAMs were isolated from E0771 tumors. All measurements (n) are biological replicates.

FIGS. 49A-49B. DNA nanodevice uptake and stability. FIG. 49A, Schematic of various fluorescently labeled nucleic acid structures used for uptake studies in BMDMs. Each nucleic acid scaffold is either a single stranded or double stranded 38 mer DNA or RNA sequence. Each scaffold is labelled with an Alexa Fluor® 647 fluorophore on the 5′ end of one of the strands. FIG. 49B, Uptake of various types of nucleic acids by M2 BMDMs. n=3/group. Statistical significance was calculated via two-tailed Student's t-test (p<0.05 values are provided); error bars indicate the mean of independent experiments±s.e.m. ns; not significant. All measurements (n) are biological replicates.

FIGS. 50A-50E. Effects of E64-DNA on the functional properties of TAMs. FIG. 50A, Catalytic activity assays for lysosomal cysteine proteases (CTSB, CTSL; 5 nM) or aspartic proteases (CTSD, CTSE; 5 nM) in the presence of vehicle (Veh; PBS) or E64-DNA (25 nM). Results are plotted as fluorescence intensity at time t, relative to time 0 (1/Io). n=3/group. FIG. 50B-50D, TAMs isolated from E0771 tumors were treated with vehicle (Veh; PBS), DNA, E64, or E64-DNA (100 nM). FIG. 50B, Cell viability (Calcein-AM) following a 72h exposure. n=4/group. FIG. 50C, CTSB and CTSL protein levels following a 24h exposure. Experiment was performed once with n=3/group. FIG. 50D, Relative mRNA levels of autophagy genes following a 24h exposure. n=3/group. FIG. 50E, LC3B and p62 protein levels in DNA or E64-DNA (10 μM) treated TAMs following a 24h treatment with vehicle (Veh; H₂O) or chloroquine (CQ, 50 μM). Representative of 2 independent experiments. Statistical significance was calculated via two-tailed Student's t-test (p<0.05 values are provided); error bars indicate the mean of independent experiments±s.e.m. ns; not significant. All measurements (n) are biological replicates.

FIGS. 51A-51B. Effects of E64-DNA on the functional properties of TAMs. FIG. 51A, Effect of E64-DNA (2h) on TBK and IRF3 phosphorylation. TAMs treated with 3′3′-cGAMP (10 μg/mL, 6h) were used as a positive control for STING activation Representative of 2 independent experiments. FIG. 51B, Effect of E64-DNA (24h) on M1- and M2-associated gene expression. n=3/group.

FIG. 52. Experimental design of antigen-cross presentation by TAMs treated with OVA or OVA₂₅₇₋₂₆₄.

FIG. 53A-53C. A lysosome-targeted DNA nanodevice (E64-DNA) promotes antigen cross-presentation by TAMs. Effect of E64-DNA on antigen cross-presentation by TAMs pre-treated with E64-DNA, DNA, or E64 (100 nM) for 2h, followed by treatment with OVA protein or OVA₂₅₇₋₂₆₄ peptide for 3h. Quantification of WWI-bound OVA₂₅₇₋₂₆₄ on TAMs (FIG. 53A). OT-1 CD8⁺ T-cell activation (FIG. 53B) and proliferation (FIG. 53C) after 72h of co-culture with TAMs. n=3/group. Vehicle (Veh)=phosphate-buffered saline. Statistical significance was calculated via two-tailed Student's t-test (p<0.05 values are provided); error bars indicate the mean of independent experiments±s.e.m. ns; not significant. TAMs were isolated from E0771 tumors. All measurements (n) are biological replicates.

FIGS. 54A-54B. E64-DNA does not activate T cells through allostimulation or direct stimulation. Control for allostimulation. CD8⁺ T cell activation (FIG. 54A) and proliferation (FIG. 54B) after 72h of co-culture with E64-DNA-treated (100 nM) TAMs that had not been exposed to antigen. CD3/CD28 antibodies were included as a positive control for T cell activation. n=3/group. Statistical significance was calculated via two-tailed Student's t-test (p<0.05 values are provided); error bars indicate the mean of independent experiments±s.e.m. All measurements (n) are biological replicates.

FIGS. 55A-55G. Inhibiting aspartic protease activity in the lysosome has minimal effect on antigen cross-presentation by macrophages. FIG. 55A, PepA-DNA design: one strand is conjugated with PepA on its 5′ end and the other with Alexa Fluor 647 to monitor uptake. FIG. 55B, Catalytic activity assays for lysosomal cysteine proteases (CTSB, CTSL; 5 nM) or aspartic proteases (CTSD, CTSE; 5 nM) in the presence of vehicle (Veh; PBS) or PepA-DNA (25 nM). Results are plotted as fluorescence intensity at time t, relative to time 0 (1/Io). n=3/group. FIGS. 55C-55F, Peritoneal macrophages were isolated and treated with vehicle (Veh; PBS), DNA, PepA, or PepA-DNA (100 nM) for the indicated times and various functional endpoints were measured. FIG. 55C, Effect of PepA-DNA (2h) on DQ-OVA degradation. n=3/group. FIG. 55D, Quantification of MHCI-bound OVA₂₅₇₋₂₆₄ on peritoneal macrophages 3h post treatment with OVA protein or OVA₂₅₇₋₂₆₄ peptide. n=3/group. FIGS. 55E-55F, pMel-CD8⁺ T cell activation (FIG. 55E) and proliferation (FIG. 55F) after 72h of co-culture with peritoneal macrophages pre-stimulated with irradiated B16F10 cells (irrB16). n=3/group. Statistical significance was calculated via two-tailed Student's t-test (p<0.05 values are provided); error bars indicate the mean of independent experiments±s.e.m. ns; not significant. All measurements (n) are biological replicates. FIG. 55G, B16.0VA tumor volume in PepA-DNA treated mice. n=9-10/group.

FIGS. 56A-56F. E64-DNA does not improve MHCII-restricted antigen presentation. Effect of E64-DNA on MHCII-restricted antigen presentation by TAMs (isolated from E0771 tumors) pre-treated with E64-DNA, DNA, or E64 (100 nM) for 2h. FIGS. 56A-56D, TAMs were incubated with OVA protein or OVA₃₃₂₋₃₃₉ peptide for 3h. OT-2 CD4⁺ T-cell activation (FIGS. 56A-56B) and proliferation (FIGS. 56C-56D) after 72h of co-culture with TAMs. n=3/group. FIGS. 56E-56F, TAMs were incubated with irradiated B16F10 cells (irrB16) or TRP1₁₁₃₋₁₂₆ peptide for 3h. TRP1 CD4⁺ T-cell activation (FIG. 56E) and proliferation (FIG. 56F) after 72h of co-culture with TAMs. n=3/group. Statistical significance was calculated via two-tailed Student's t-test (p<0.05 values are provided); error bars indicate the mean of independent experiments±s.e.m. All measurements (n) are biological replicates.

FIG. 57. Experimental design of intratumoral delivery (i.t.). b-e, DNA or E64-DNA (25 μg) were injected intratumorally into E0771 tumors.

FIGS. 58A-58C. The E64-DNA nanodevice preferentially localizes in lysosomes of M2-like TAMs and lowers tumor growth. FIG. 58A, Flow cytometry analysis of E64-DNA uptake by various tumor cell types 7h after injection. n=3/group. FIG. 58B, Representative images (left) and Pearson correlation (right) of co-localization of lysotracker labeled lysosomes (green) with E64-DNA (red). Pearson correlation with and without a 20-pixel shift (˜lysosome diameter) of the green signal. n=12 cells/group. scale bar=10 μm. FIG. 58C, DQ-OVA degradation by TAMs isolated from tumors 7h after injection. n=3/group. Statistical significance was calculated via two-tailed Student's t-test (p<0.05 values are provided); error bars indicate the mean of independent experiments±s.e.m. #, FDR<5% G-test and t-test (shotgun proteomics analyses). Neg=unlabeled negative control. All measurements (n) are biological replicates.

FIG. 59. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. TAMs from E0771 tumors 7h after mice were treated with DNA or E64-DNA (25 μg, i.t.) (corresponds to FIG. 58C).

FIG. 60. The E64-DNA nanodevice preferentially localizes in lysosomes of M2-like TAMs and lowers tumor growth. Flow cytometric analysis of E64-DNA uptake by CD206^{high} or CD206^{low} TAMs 7h after injection. Representative flow images of CD206 gating (left) and quantification (right) are shown. n=3/group. Statistical significance was calculated via two-tailed Student's t-test (p<0.05 values are provided); error bars indicate the mean of independent experiments±s.e.m. #, FDR<5% G-test and t-test (shotgun proteomics analyses). Neg=unlabeled negative control. All measurements (n) are biological replicates.

FIG. 61A-61C. DNA nanodevice uptake and stability. FIG. 61A, Schematic of an E64-DNA uptake competition assay in M1 and M2 BMDMs. FIG. 61B, Hoechst dye levels in individually cultured M1 and M2 BMDMs. FIG. 61C, E64-DNA uptake by co-cultured M1 and M2 BMDMs. Representative flow cytometry data (left) and quantification (right) are shown. n=3/group. Statistical significance was calculated via two-tailed Student's t-test (p<0.05 values are provided); error bars indicate the mean of independent experiments±s.e.m. ns; not significant. All measurements (n) are biological replicates.

FIG. 62. The E64-DNA nanodevice preferentially localizes in lysosomes of M2-like TAMs and lowers tumor growth. Scavenger receptor levels (quantified by spectral counts) in M1-like and M2-like TAMs from E0771 tumors. n=5/group.

FIG. 63. The E64-DNA nanodevice preferentially localizes in lysosomes of M2-like TAMs and lowers tumor growth. E64-DNA was injected intratumorally into E0771 tumors. Flow cytometry analysis of E64-DNA uptake by TAMs 7h after injection. n=4/group. Statistical significance was calculated via two-tailed Student's t-test (p<0.05 values are provided); error bars indicate the mean of independent experiments±s.e.m. #, FDR<5% G-test and t-test (shotgun proteomics analyses). Neg=unlabeled negative control. All measurements (n) are biological replicates.

FIGS. 64A-64C. The E64-DNA nanodevice preferentially localizes in lysosomes of M2-like TAMs and lowers tumor growth. E64-DNA was injected intratumorally into E0771 tumors. Flow cytometry analysis of DQ-OVA degradation (FIG. 64A) by TAMs 7h after injection. n=4/group. E0771 tumor volume 5 days after injection (FIG. 64B). n=5/group. FIG. 64C, E64, DNA, or E64-DNA (25 μg) were injected into E0771 tumors and tumor volume was assessed 5 days after injection. Veh and DNA: n=8/group, E64: n=9/group, E64-DNA: n=7/group. Statistical significance was calculated via two-tailed Student's t-test (p<0.05 values are provided); error bars indicate the mean of independent experiments±s.e.m. #, FDR<5% G-test and t-test (shotgun proteomics analyses). Neg=unlabeled negative control. All measurements (n) are biological replicates.

FIG. 65. The E64-DNA nanodevice preferentially localizes in lysosomes of M2-like TAMs and lowers tumor growth. Effect of E64-DNA on E0771 cell proliferation in vitro. n=6/group. Vehicle (Veh)=phosphate-buffered saline.

FIGS. 66A-66B. Intravenously delivered E64-DNA targets TAMs to activate CD8⁺ T cells and attenuate tumor growth. E64-DNA or DNA (25 μg) was intravenously delivered (i.v.; retro-orbital) into E0771 tumor-bearing mice. FIG. 66A, Flow cytometry analysis of E64-DNA uptake by various tumor cell types 7h after a single injection is shown. n=3/group. FIG. 66B, DQ-OVA degradation by TAMs isolated from tumors 7h after a single injection is shown. n=3/group. Statistical significance was calculated via two-tailed Student's t-test (p<0.05 values are provided); error bars indicate the mean of independent experiments±s.e.m. ns, not significant. All measurements (n) are biological replicates

FIG. 67. Representative flow cytometry analyses of DQ-OVA degradation and cysteine protease activity (ProSense 680). Representative flow cytometry data on DQ-OVA degradation assays performed on macrophages from a variety of sources and genotypes. Neg=unlabeled negative control. TAMs isolated from E0771 tumors 7h after mice were treated with DNA or E64-DNA (25 μg, i.v.) (corresponds to FIG. 66B).

FIG. 68. DNA nanodevice uptake and stability. Native polyacrylamide gel of dsDNA incubated in 100% mouse serum for various time points. Intact dsDNA was quantified by densitometry. Representative of 2 independent experiments.

FIG. 69. Intravenously delivered E64-DNA targets TAMs to activate CD8⁺ T cells and attenuate tumor growth. E64-DNA or DNA (25 μg) was intravenously delivered (i.v.; retro-orbital) into E0771 tumor-bearing mice. E0771 tumor growth over 5 days after a single injection is shown. n=8/group. Statistical significance was calculated via two-tailed Student's t-test (p<0.05 values are provided); error bars indicate the mean of independent experiments±s.e.m. ns, not significant. All measurements (n) are biological replicates.

FIG. 70. Intravenously delivered E64-DNA targets TAMs to activate CD8⁺ T cells and attenuate tumor growth. E64-DNA or DNA (25 μg) was intravenously delivered (i.v.; retro-orbital) into E0771 tumor-bearing mice. Immune cell composition (n=8/group) from E0771 tumors 5 days after a single injection is shown. Statistical significance was calculated via two-tailed Student's t-test (p<0.05 values are provided); error bars indicate the mean of independent experiments±s.e.m. ns, not significant. All measurements (n) are biological replicates.

FIG. 71. Gating strategy and representative flow cytometry data for tumor immune cell composition. Representative flow cytometry data for immune cell composition in E0771 tumors, 5 days after a single injection of DNA or E64-DNA (25 μg, i.v.).

FIG. 72. Intravenously delivered E64-DNA targets TAMs to activate CD8⁺ T cells and attenuate tumor growth. E64-DNA or DNA (25 μg) was intravenously delivered (i.v.; retro-orbital) into E0771 tumor-bearing mice. CD8⁺ T cell activation and proliferation status (DNA: n=8/group, E64-DNA: n=7/group) from E0771 tumors 5 days after a single injection is shown. Statistical significance was calculated via two-tailed Student's t-test (p<0.05 values are provided); error bars indicate the mean of independent experiments±s.e.m. ns, not significant. All measurements (n) are biological replicates.

FIGS. 73A-73B. E64-DNA does not activate T cells through allostimulation or direct stimulation. FIGS. 73A-73B, Control for direct effects of E64-DNA on T cells. CD8⁺ T cell activation (FIG. 73A) and proliferation (FIG. 73B) after 72h of culturing in complete growth media (Media) in the presence/absence of E64-DNA (100 nM). CD3/CD28 antibodies were included as a positive control for T cell activation. n=3/group. Statistical significance was calculated via two-tailed Student's t-test (p<0.05 values are provided); error bars indicate the mean of independent experiments±s.e.m. All measurements (n) are biological replicates.

FIG. 74. Experimental design for depleting TAMs with α-CSF1R antibody (top). Effect of IgG or α-CSF1R (300 μg) on E0771 tumor growth (bottom, left) and CD8⁺ effector T cells in tumors (bottom, right) in mice treated with E64-DNA (n=8/group) or DNA (n=6/group). E64-DNA or DNA (25 μg) was intravenously delivered (i.v.; retro-orbital) into E0771 tumor-bearing mice.

FIGS. 75A-75B. Intravenously delivered E64-DNA targets TAMs to activate CD8⁺ T cells and attenuate tumor growth. E64-DNA or DNA (25 μg) was intravenously delivered (i.v.; retro-orbital) into E0771 tumor-bearing mice. FIG. 75A-75B, Linear regression of % CD8⁺ effector T cells in tumors vs. tumor volume in DNA or E64-DNA treated mice (FIG. 75A, n=8/group), and in E64-DNA treated mice treated with IgG (n=8) or α-CSF1R (n=6) antibodies (FIG. 75B) is shown. Statistical significance was calculated via two-tailed Student's t-test (p<0.05 values are provided); error bars indicate the mean of independent experiments±s.e.m. ns, not significant. All measurements (n) are biological replicates.

FIGS. 76A-76B. Effects of α-CD8, α-PD-L1, or IgG antibodies on E0771 tumor growth in mice treated with E64-DNA or DNA. E64-DNA or DNA (25 μg) was intravenously delivered (i.v.; retro-orbital) and anti-CD8 (FIG. 76A) or IgG control antibody (200 μg) or anti-PD-L1 (FIG. 76B) or IgG control antibody (100 μg) was intraperitoneally delivered into E0771 tumor-bearing mice. n=5/group.

FIG. 77. Intravenously delivered E64-DNA targets TAMs to activate CD8⁺ T cells and attenuate tumor growth. a-k, E64-DNA or DNA (25 μg) was intravenously delivered (i.v.; retro-orbital) into E0771 tumor-bearing mice. Antigen cross-presentation (OVA-0T-1 system) by pooled TAMs from E0771 tumors of DNA or E64-DNA-treated mice (top, n=6/group), and M1-like and M2-like sorted TAMs from E0771 tumors followed by DNA or E64-DNA-treatment ex vivo (bottom, n=3/group). Statistical significance was calculated via two-tailed Student's t-test (p<0.05 values are provided); error bars indicate the mean of independent experiments±s.e.m. ns, not significant. All measurements (n) are biological replicates.

FIG. 78A-78E. E64-DNA attenuates tumor growth and improves antigen cross-presentation by TAMs in the B16.OVA model. FIG. 78A, Experimental design (left). Effect of E64-DNA (25 μg, i.v.) on B16.0VA tumor growth (right). n=8/group. FIG. 78B, OT-1-CD8⁺ T cell activation (left) and proliferation (right) after 72h of co-culture with TAM isolated from DNA or E64-DNA (i.v.) treated B16.0VA tumors. n=6/group. FIG. 78C, pMel-CD8⁺ T cell activation (left) and proliferation (right) after 72h of co-culture with TAMs isolated from DNA or E64-DNA (i.v.) treated B16.0VA tumors. n=6/group. FIGS. 78D-78E, Effects of E64-DNA on CD8⁺ T cell activation and proliferation status 5 days after a single injection. Representative flow images (FIG. 78D) and quantification (FIG. 78E). n=9/group. Statistical significance was calculated via two-tailed Student's t-test (p<0.05 values are provided); error bars indicate the mean of independent experiments±s.e.m. All measurements (n) are biological replicates.

FIG. 79. Intravenously delivered E64-DNA targets TAMs to activate CD8⁺ T cells and attenuate tumor growth. E64-DNA or DNA (25 μg) was intravenously delivered (i.v.; retro-orbital) into E0771 tumor-bearing mice. Experimental design (top). Effect of E64-DNA (25 μg) and cyclophosphamide (CTX, 50 mg/kg), alone or in combination, on E0771 tumor growth (bottom). n=6/group. Vehicle (Veh)=phosphate-buffered saline. Statistical significance was calculated via two-tailed Student's t-test (p<0.05 values are provided); error bars indicate the mean of independent experiments±s.e.m. ns, not significant. All measurements (n) are biological replicates.

FIG. 80. Model of how E64-DNA targets TAM to promote anti-tumor immunity.

FIGS. 81A-81C. T0901317-DNA attenuates atherosclerotic lesion development. Low Density Lipoprotein Receptor negative (Ldlr−/−) mice were fed a Western-type diet for 6 weeks to create atherosclerotic lesions. After the 6 weeks, mice were treated with DNA (50 μg) or T0901317-DNA (TO-DNA: 50 μg DNA, 1.9 μg T0901317) once/day, 5 days/week, intravenously for 4 weeks. FIG. 81A, Atherosclerotic lesions were quantified in the aortic root and innominate artery. FIG. 81B, Plasma cholesterol and triglyceride levels. FIG. 81C, Body weight. Results are mean±SEM. *p<0.05 t-test, n=9-10/group.

FIG. 82. GNE-DNA attenuates hypoxia-induced lactate production by macrophages (lactate production: infection, cancer). Bone marrow-derived macrophages (BMDMs) were cultured under normoxic (n) or hypoxic (h, 1% 02) conditions for 24h in the presence of vehicle (veh), GNE, or GNE-DNA. Lactate dehydrogenase (LDH) activity in BMDMs is decreased by GNE or GNE-DNA under hypoxic conditions to levels comparable to normoxic conditions compared to vehicle. Results are mean±SEM. *p<0.05 (t-test, relative to vehicle), ns: not significant, n=4.

FIG. 83. GNE-DNA attenuates hypoxia-induced lactate production by macrophages (lactate production: infection, cancer). Bone marrow-derived macrophages (BMDMs) were cultured under normoxic (n) or hypoxic (h, 1% 02) conditions for 24h in the presence of vehicle (veh), GNE, or GNE-DNA. Intracellular lactate levels in BMDMs are decreased by GNE or GNE-DNA under hypoxic conditions to levels compared to vehicle. Results are mean±SEM. *p<0.05 (t-test, relative to vehicle), ns: not significant, n=4.

FIG. 84. Ibrutinib-DNA attenuates inflammation in adipose tissue macrophages (ATMs) from obese mice (anti-inflammatory: metabolic disease). Relative mRNA levels of inflammatory and lipid metabolism genes in ATMs purified from epididymal fat of obese male C57BL/6 mice fed a 60% high fat diet (HFD). ATMs were treated with indicated concentrations of Ibrutinib or Ibrutinib-DNA for 6 hours. Results are mean±SEM. *p<0.05 (t-test, relative to vehicle), ns: not significant, n=4.

FIG. 85. GW3965-DNA enhances lipid metabolism gene expression in macrophages (LXR agonist: metabolic disease). Bone marrow-derived macrophages (BMDMs) were treated with vehicle, DNA (5 GW395 (5 or GW3965-DNA (5 μM) for 24 h and relative gene expression was quantified by qRT-PCR. Results are mean±SEM. *p<0.05 (t-test, relative to vehicle), ns: not significant, n=3.

FIG. 86. Schematic for addressing disease via nucleic acid-derivatized therapeutics.

FIG. 87. BMDM uptake of nucleic acid derivatized magnetic labels. Flow cytometric analysis (upper panel revealed that derivatization of nucleic acid targeting modules with magnetic labels (iron oxide, Probe 1 and gadolinium, Probe 3, lower panel) did not impede uptake by macrophages as compared to unlabeled nucleic acid targeting modules.

FIGS. 88A-88B. MRI imaging of ex vivo E0771 tumors injected with nucleic acid derivatized magnetic labels. Both nucleic acid derivatized imaging agents, Probe 1 (iron oxide, FIG. 88A) and Probe 3 (gadolinium, FIG. 88B) were visible via MRI imaging after intratumoral injection. Arrows point to injection sites, darker regions show accumulation of Mill agents (greyish-black regions).

FIG. 89. Intravenously administered nucleic acid derivatized MRI imaging agents accumulate in E0771 tumors in vivo. Uptake into tumor is indicated by the black arrows, and into the bladder is indicated by grey arrows. Strong intratumoral signal of gadolinium was apparent 2 h post IV administration of Probe 3 (middle image). Gadolinium signal was still evident at 4 h post IV administration (right image). These results indicate that tumors can be readily viewed via MRI using nucleic acid derivatized imaging agents, such as gadolinium, for extended periods of time after administration of the imaging agent.

FIG. 90A-90B. Time course of intratumoral imaging agent accumulation. The time course of accumulation of gadolinium signal (FIG. 90A) in region of interested in a selected slice of a tumor shown in FIG. 90B over time after DNA complex injection. Gadolinium signal reached a maximum by 20 mins and remained stable through the course of the experiment.

FIG. 91. Intravenously administered nucleic acid derivatized MRI imaging agents accumulate in atherosclerotic lesions in vivo. A gradient echo anatomy reference (left image) shows the location of the kidneys (arrows) and the dynamic contrast enhanced MRI image of the same slice (right image) demonstrates uptake of the gadolinium-DNA in the atherosclerotic lesion in the descending artery in the renal area (bright region marked by the arrow). The asymmetry of the lesions in the artery wall are consistent with the hemodynamics of blood flow mediating the site of lesion formation along the artery wall.

### DETAILED DESCRIPTION OF PREFERRED EMBODIMENTS

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

All publications, patents and patent applications cited herein are hereby expressly incorporated by reference in their entirety for all purposes.

### Definitions

Before describing the methods and compositions of the disclosure in detail, a number of terms will be defined. As used herein, the singular forms “a,” “an,” and “the” include plural referents unless the context clearly dictates otherwise. For example, reference to “a therapeutic target” means one or more therapeutic targets.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “oligonucleotide” is used interchangeably with “nucleic acid molecule” and is understood to be a molecule that has a sequence of nucleic acid bases that can include monomer units at defined intervals. For example, an oligonucleotide can include a molecule including two or more nucleotides.

As used herein, the terms “complementary” or “complementarity,” when used in reference to nucleic acids (i.e., a sequence of nucleotides such as an oligonucleotide), refer to sequences that are related by base-pairing rules.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

As used herein, the terms “therapeutic amount,” “therapeutically effective amount” or “effective amount” can be used interchangeably and refer an amount of a compound that becomes available through an appropriate route of administration to provide a therapeutic benefit to a patient for a disorder, a condition, or a disease. The amount of a compound which constitutes a “therapeutic amount,” “therapeutically effective amount” or “effective amount” will vary depending on the compound, the disorder and its severity, and the age of the subject to be treated, but can be determined routinely by one of ordinary skill in the art.

“Treating” or “treatment,” as used herein, covers the treatment of a disorder, condition, or a disease described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing development or exacerbation of, providing symptomatic relief for, or curing a patient's disorder, condition, or disease.

As used herein, the terms “patient,” “subject,” and “individual” can be used interchangeably and refer to an animal. For example, the patient, subject, or individual can be a mammal, such as a human to be treated for a disorder, condition, or a disease.

As used herein, the terms “disorder,” “condition,” or “disease” refer, for example, to cancers and associated comorbidities, as well as metabolic diseases, obesity, insulin resistance, diabetes, coronary heart disease, atheroschlerosis, hyperlipidemia, and hypertriglyceridemia.

It is noted that terms like “preferably,” “commonly,” and “typically” are not utilized herein to limit the scope of the methods and compositions as described herein or to imply that certain features are critical, essential, or even important to the structure or function of the subject matter recited in the claims.

As used herein, the term “cancer” refers to any type of cancerous cell or tissue as well as any stage of a cancer from precancerous cells or tissues to metastatic cancers. For example, as used herein, cancer can refer to a solid cancerous tumor, leukemia, and/or a neoplasm.

### Overview

Provided herein are therapeutic compositions and methods for treating a subject by modulating cell populations using the therapeutic compositions. The therapeutic compositions can include a nucleic acid targeting module and a therapeutic agent associated with the targeting module. The nucleic acid targeting module targets the therapeutic to the lysosome of a macrophage. The therapeutic compositions can be used to treat diseases, such as cancer, atherosclerosis, diabetes, obesity, hyperlipidemia, and others. The therapeutic compositions provided herein can also include i) a DNA targeting platform, comprising a double-stranded DNA targeting module and a cathepsin inhibitor and a secondary therapeutic agent. Also provided herein are therapeutic compositions comprising a DNA targeting platform comprising a double-stranded DNA targeting module and a neoantigen.

Also provided herein are various methods of administering therapeutic compositions to subjects in need thereof. The methods can include a method of treating cancer in a subject. The method can include administering to the subject a therapeutic composition comprising a nucleic acid targeting module attached to a cathepsin inhibitor. The nucleic acid molecule targets the cathepsin inhibitor to the lysosome of a tumor associated macrophage (TAM). The methods can also include a method of administering a therapeutic agent to a subject. The method comprises providing a therapeutic construct comprising a therapeutic agent attached to a nucleic acid targeting module, wherein the nucleic acid targeting module targets the therapeutic agent to the lysosome of a macrophage and administering the therapeutic construct to the subject. The therapeutic agent is released from the lysosome of the macrophage upon degradation of the nucleic acid targeting module. The methods can further include a method of minimizing side effects of a therapeutic agent comprising conjugating a therapeutic agent to a nucleic acid targeting module that targets the nucleic acid targeting module to the lysosome of a macrophage, administering the conjugated therapeutic agent to a subject, and releasing the therapeutic agent from the lysosome of the macrophage upon degradation of the targeting module. The therapeutic agent is released into the cytosol, nucleus, and/or immediate extracellular microenvironment of the macrophage and minimizes side effects of the therapeutic agent. These and other therapeutic compositions and methods are contemplated herein

A DNA-based nanodevice preferentially delivers drugs to macrophages in vivo. A DNA-based nanodevice has been developed to preferentially target macrophages in vivo. The DNA-based nanodevice can comprise, for example, two or three modules: i) a macrophage targeting module, or targeting module (e.g., polyanionic DNA) which enables preferential uptake of the nanodevice by macrophages, ii) a therapeutic module (comprising one or more drugs, also referred to as a therapeutic load module) which enables targeting of specific pathway(s) in macrophages, and/or iii) a labeling module (e.g., a molecule that enables measurement and/or quantification of nanodevice uptake, such as a fluorophore or other detectable molecule).

The polyanionic backbone of DNA makes it an ideal ligand for scavenger receptors, which are present abundantly on macrophages, enabling targeting of the nanodevice to lysosomes via endocytosis. The DNA backbone is degraded in the lysosome, thereby liberating the therapeutic module (e.g., a small molecule or peptide drug). For drug targets within the lysosome, this serves as an ideal method of delivery. However, because membrane-soluble drugs can diffuse out of the lysosome, this approach can also be used to reach targets in other subcellular compartments, such as the cytosol, nucleus, etc., and/or the immediate extracellular microenvironment of the macrophage. Because of the specific targeting and regiospecific release mechanism employed by the therapeutic construct, it is believed that therapeutic agents with problematic side-effects when delivered systemically can be effectively administered to individuals with minimized side-effects.

The specificity, modularity, and trackability of this DNA-based nanodevice are significant improvements over existing technologies. The DNA-based nanodevice i) targets preferentially macrophages in multiple tissues, allows for delivery of drugs that target lysosomal and cytosolic proteins, and iii) enables manipulation of macrophage functions.

The DNA-based nanodevice can confer therapeutic activity to molecules that are otherwise not effective. As shown herein, the DNA-based nanodevice confers therapeutic properties to a lysosomal cysteine protease (LCP) inhibitor (E64) in tumor models. Elevated tumor LCP levels are a poor prognostic marker for a wide range of solid tumors, including triple negative breast cancer, colorectal cancer, lung cancer, ovarian cancer, pancreatic adenocarcinoma, amongst others. Despite this strong association, high doses of E64 (1 mg, daily) had minimal impact on tumor growth in murine cancer models. More recently, activity-based probes were used to show that the majority of tumor LCP activity is tumor-associated macrophage (TAM)-associated. However, the contribution of TAM LCP activity to tumor growth is unknown.

It was recently discovered that elevated LCP activity in TAMs blocks their ability to cross-present tumor-derived antigens to activate CD8⁺ T cells, which in turn, promotes tumor development. Because E64 has a limited ability to cross cell membranes and lacks selectivity to TAMs, it was reasoned that an E64-DNA construct might produce a therapeutic response by overcoming these hurdles. E64 was therefore conjugated to a DNA-based nanodevice to create E64-DNA. Unlike free E64, E64-DNA preferentially targeted TAMs in vivo. E64-DNA improved antigen cross-presentation by TAMs and attenuated tumor growth via CD8⁺ T cells in triple-negative breast cancer (TNBC), lung, and melanoma models. When combined with cyclophosphamide, a frontline chemotherapy, E64-DNA showed sustained tumor regression in a TNBC model. These findings underscore the power of the DNA-based nanodevice to deliver drugs that preferentially target macrophages and manipulate their functions for therapeutic value.

In some embodiments of the present disclosure, the DNA-based nanodevice causes reprogramming of target macrophages. For example, in some embodiments, the DNA-based nanodevice reduces the lysosomal degradative capacity of TAMs. In some embodiments, the DNA-based nanodevice modulates macrophage function and/or takes advantage of macrophage phagocytic mechanisms without killing the target macrophages to deliver a therapeutic module.

In some embodiments of the present disclosure, the targeting module can target the therapeutic module to a specific organelle within a macrophage. In some embodiments, the targeting module can target the therapeutic module to the lysosome of a macrophage. In some embodiments, the therapeutic module targeted to the lysosome can act on target molecules outside of the lysosome, either in another intracellular compartment, in the cytosol, or in the immediate surroundings of the macrophage. In some embodiments, therapeutic modules can be liberated from targeting modules, for example, by degradation of the targeting modules in the endosomal pathway, resulting in subsequent untargeted distribution of the therapeutic module from the targeted destination.

Contemplated targeting, therapeutic, and labeling modules are described below.

### Targeting Modules

The targeting modules of the present disclosure are designed to be recognized by a cell type within the body, i.e., macrophages. In some embodiments, the targeting modules are designed to be recognized by a specific population of macrophages. In some embodiments, the targeting modules are recognized by tumor-associated macrophages. In some embodiments, the targeting modules are recognized by alveolar macrophages. In some embodiments, the targeting modules are recognized by adipose tissue macrophages. In some embodiments, the targeting modules can be nucleic acid molecules.

A nucleic acid molecule can have a sequence of bases on a backbone that form an oligonucleotide. The most common oligonucleotides have a backbone of sugar phosphate units. A distinction can be made between oligodeoxyribonucleotides, which do not have a hydroxyl group at the 2′ position, and oligoribonucleotides, which have a hydroxyl group in this position. Oligonucleotides also can include derivatives, in which the hydrogen of the hydroxyl group is replaced with organic groups, e.g., an allyl group. An oligonucleotide is a nucleic acid that includes at least two nucleotides.

One nucleic acid sequence may be complementary to a second nucleic acid sequence in that the two strands anneal to one another under certain conditions according to base pairing rules. For natural bases, the base pairing rules are those developed by Watson and Crick. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of annealing between the nucleic acid strands.

Oligonucleotides, as described herein, can be capable of forming hydrogen bonds with oligonucleotides having a complementary base sequence. These bases can include the natural bases such as A, G, C, T and U, as well as artificial bases. An oligonucleotide can include nucleotide substitutions. For example, an artificial or modified base can be used in place of a natural base such that the artificial base exhibits a specific interaction that is similar to the natural base.

In one embodiment, targeting modules contemplated herein can be double-stranded or single-stranded RNA, DNA, and variations thereof. Examples include, but are not limited to, single-stranded ribonucleic acid (ssRNA), single-stranded deoxyribose nucleic acid (ssDNA), double-stranded RNA (dsRNA), double-stranded DNA (dsDNA), modified RNA, or RNA/DNA complexes. In some embodiments, the nucleic acid sequences are designed to be recognized by one or more populations of scavenger receptors expressed on macrophages.

The embodiments and examples herein discussing a dsDNA targeting module are contemplated to be equally applicable to ssRNA targeting modules and vice versa. Therefore, the use of the term “DNA-based nanodevice” is not intended to limit the targeting modules contemplated herein to only DNA-based constructs, but rather to indicate any nucleic acid targeting module, with or without chemical modifications to the backbone and nucleobases.

In one specific embodiment, the targeting module is a double-stranded deoxyribose nucleic acid (dsDNA). dsDNA targeting modules can include one or more DNA sequences that complex together to form a dsDNA structure. Each strand of the dsDNA structure can have any desired length irrespective of its complementary strand in the structure. For example, in the context of a two stranded dsDNA targeting module each of the first and second single-stranded nucleic acid molecules can have a length of between about 20 to about 100 nucleotides. In one embodiment, a dsDNA targeting module can have two strands that are partially or fully complementary to each other.

While not wishing to be bound by theory, it is believed that from an uptake perspective ssDNA, ssRNA, and dsDNA can be equivalent. However, dsDNA offers many advantages from the perspective of greater stability, greater adaptability to delivering multiple therapeutics (e.g., multiple different therapeutic agents can be attached to a dsDNA targeting module), and greater adaptability to carrying/delivering multiple tracking molecules and/or devices, as described herein elsewhere. For example, a dsDNA targeting module can have 1, 2, 3, 4, 5, 6, 7, 8, 9, or 10 different therapeutic agents and/or tracking molecules attached thereto. Moreover, ssDNA and ssRNA can be more likely to be enzymatically degraded in the blood stream, and thus, can be less efficient targeting modules. Further, ssDNA can be more likely to be immunogenic, as can ssRNA (depending on the sequence). In the present disclosure, studies with dsDNA were surprising in three regards: 1) it was found that dsDNA was not degraded by DNAses on timescales that would prevent using it for targeting; 2) targeting with dsDNA did not need to be selective (indicating the relative abundance of receptors on macrophages over other competing cells); and 3) the immunogenicity of dsDNA constructs was negligible or low enough to be inconsequential.

In some embodiments, the nucleic acid sequence includes one or more alternative nucleic acids. An alternative nucleic acid can comprise a natural modified base, an unnatural modified base, a base analog, or a synthetic derivative of a nucleobase. An alternative nucleic acid can be a nucleic acid analog. In some embodiments, the natural modified base is selected from the group comprising 6-keto purine, xanthine, 5-methylcytosine, and 2-aminopurine; the unnatural modified base can be selected from group comprising thioguanine, 8-oxoguanine, deazapurine, and azapurine; the base analog can be selected from group comprising nebularin, nitroindole, and nitropyrrole derivative; the synthetic derivative of a nucleobase can be selected from group comprising a bromo-substituted derivative and a fluoro-substituted derivative; and the nucleic acid analog can be selected from group comprising Peptide Nucleic Acid (PNA), Locked Nucleic Acid (LNA), morpholino, methyl phosphonate, phosphorothioate, and 2′-O-modified oligonucleotide.

In one embodiment, a therapeutic composition of the present invention includes a targeting module that is about 38 base pairs in length and a therapeutic module associated (for example, permanently or temporarily attached and/or directly or indirectly attached) with the targeting module.

In some embodiments, the nucleic acid targeting module comprises a first single-stranded nucleic acid molecule and a second single-stranded nucleic acid molecule that is partially or fully complementary to the first single-stranded molecule. It is known in the art that constructs with fewer than 15 bases have a low melting temperature: strands can fall apart at body temperature. Further, errors in DNA synthesis can go up substantially above for strands above 100 bases in length (and longer constructs are costlier to produce). Constructs with more than 500 bases can have too much DNA for too little drug. Therefore, in some embodiments, each of the first and second single-stranded nucleic acid molecules is between 15 and 500 nucleotides in length. In some embodiments, each of the first and second single-stranded nucleic acid molecules is between 30 and 50, or between 20 and 60, nucleotides in length. In one embodiment, the dsDNA targeting module includes a first single-stranded nucleic acid molecule that includes the nucleic acid sequence of SEQ ID NO: 40 and a second single-stranded nucleic acid molecule that includes the nucleic acid sequence of SEQ ID NO: 41 or 42.

Any means for connecting or attaching the therapeutic module to the targeting module is contemplated herein. In some embodiments, the therapeutic module is attached to the targeting module by a covalent bond or other chemical bond. In some embodiments, the therapeutic module is conjugated to the targeting module. In some embodiments, the therapeutic module is linked to the targeting module by a linker molecule (e.g., a peptide, a nucleic acid, a small molecule, amine, dibenzocyclooctyne (DBCO), azide, one or more aliphatic carbon chain spacers, tetraethylene glycol, polyethylene glycol or other linker molecule). In other embodiments, the therapeutic module is associated with the targeting module. In one specific embodiment, one strand of the dsDNA targeting module is chemically modified with an amine group. Subsequent chemical modification of the amine group, as described herein elsewhere, can be used to form a covalent bond with the therapeutic module. In another specific embodiment, one or both strands of the dsDNA targeting module is chemically modified with a DBCO group. Subsequent chemical modification of the DBCO group with an azide group via click chemistry, as described herein elsewhere, can be used to form a covalent bond with the therapeutic module. Additional attachment means are contemplated herein, such that the therapeutic module and the targeting molecule are directly or indirectly (e.g., via a linker) attached.

Some embodiments of the present disclosure can use non-nucleic acid entities in the targeting process. Some embodiments can use non-nucleic acid entities in addition to nucleic acid entities in the targeting process. In some embodiments, therapeutic modules are further targeted through use of known ligands specific to receptors on macrophages or macrophage subsets attached to a DNA scaffold. In other embodiments, aptamers which have been generated against plasma membrane proteins of specific macrophage subsets are attached to the therapeutic modules to accomplish targeting. In some embodiments, bispecific aptamers against both scavenger receptors and another receptor present on the target macrophages are used.

### Therapeutic Modules

The present disclosure contemplates a variety of entities to comprise the therapeutic module of the DNA-based nanodevice. The therapeutic module can comprise one or more therapeutic agents. In some embodiments, the nucleic acid targeting module is linked to more than one therapeutic agent. In some embodiments, the DNA-based nanodevice comprises “stacking” of therapeutic agents, with each therapeutic agent linked to a single strand of nucleic acid and the nanodevice comprising more than one such component.

The present disclosure contemplates a variety of therapeutic modalities. In some embodiments, the one or more therapeutic agents of the therapeutic module are small molecules. In some embodiments, the one or more therapeutic agents of the therapeutic module are peptides. In some embodiments, the therapeutic module comprises both small molecules and peptides.

The present disclosure contemplates DNA-based nanodevices with therapeutic modules targeting a variety of targets. The variety of drug categories and mechanisms contemplated herein include but are not limited to the following classes and example therapeutic agents:

Cathepsin inhibitors. Cathepsins are a group of protease enzymes originally discovered in the cell lysosome, with several members ubiquitous in the human body. They are not catalytically conserved: some are serine proteases, some are aspartate proteases, and many are lysosomal cysteine proteases. Cysteine cathepsins are misregulated in a wide variety of tumors, and are involved in cancer progression, angiogenesis, metastasis, and in the occurrence of drug resistance. Contemplated cysteine protease inhibitors include E64, which is represented by Formula I below.

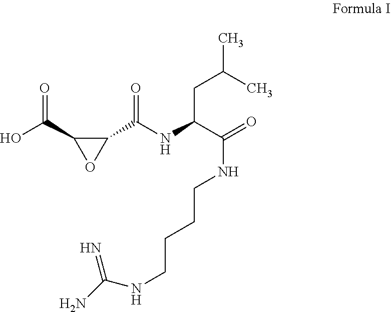 [SMILES: CC(C)C[C@H](NC(=O)[C@@H]1O[C@H]1C(=O)O)C(=O)NCCCCNC(=N)N]

Contemplated aspartic protease inhibitors include CA074. Cathepsin inhibitors contemplated by this disclosure include, but are not limited to, the following molecular entities: epoxysuccinyl peptide derivatives [E-64, E-64a, E-64b, E-64c, E-64d, CA-074, CA-074 Me, CA-030, CA-028, etc.], peptidyl aldehyde derivatives [leupeptin, antipain, chymostatin, Ac-LVK-CHO5 Z-Phe-Tyr-CHO, Z-Phe-Tyr(OtBu)-COCHO·H2O, 1-Naphthalenesulfonyl-Ile-Trp-CHO, Z-Phe-Leu-COCHO·H2O, etc.], peptidyl semicarbazone derivatives, peptidyl methylketone derivatives, peptidyl trifluoromethylketone derivatives [Biotin-Phe-Ala-fluoromethyl ketone, Z-Leu-Leu-Leu-fluoromethyl ketone minimum, Z-Phe-Phe-fluoromethyl ketone, N-Methoxysuccinyl-Phe-HOMO-Phe-fluoromethyl ketone, Z-Leu-Leu-Tyr-fluoromethyl ketone, Leupeptin trifluoroacetate, ketone, etc.], peptidyl halomethylketone derivatives [TLCK, etc.], bis(acylamino)ketone [1,3-Bis(CBZ-Leu-NH)-2-propanone, etc.], peptidyl diazomethanes [Z-Phe-Ala-CHN2, Z-Phe-Thr(OBzl)-CHN2, Z-Phe-Tyr (O-t-But)-CHN2, Z-Leu-Leu-Tyr-CHN2, etc.], peptidyl acyloxymethyl ketones, peptidyl methylsulfonium salts, peptidyl vinyl sulfones [LHVS, etc.], peptidyl nitriles, disulfides [5,5′-dithiobis[2-nitrobenzoic acid], cysteamines, 2,2′-dipyridyl disulfide, etc.], non-covalent inhibitors [N-(4-Biphenylacetyl)-5-methylcysteine-(D)-Arg-Phe-b-phenethylamide, etc.], thiol alkylating agents [maleimides, etc], azapeptides, azobenzenes, O-acylhydroxamates [Z-Phe-Gly-NHO-Bz, Z-FG-NHO-BzOME, etc.], lysosomotropic agents [chloroquine, ammonium chloride, etc.], and inhibitors based on Cystatins [Cystatins A, B, C, stefins, kininogens, Procathepsin B Fragment 26-50, Procathepsin B Fragment 36-50, etc.].

LDHA inhibitors. Lactate dehydrogenase A (LDHA) is found in the cytosol of cells in most somatic tissues. The enzyme catalyzes the inter-conversion of pyruvate and L-lactate along with regenerating NAD+-form NADH. LDHA has an aberrantly high expression in multiple cancers, which is associated with malignant progression. Contemplated LDHA inhibitors include FX11, gossypol, GSK2837808A, galloflavin, N-hydroxyindole-based inhibitors (such as NHI-2), (R)-GNE-140, AZ-33, oxamate, a quinoline 3-sulfonamide, and machilin. LDHA inhibitors contemplated by this disclosure include, but are not limited to, the following molecular entities: 3-((3-carbamoyl-7-(3,5-dimethylisoxazol-4-yl)-6-methoxyquinolin-4-yl) amino) benzoic acid, N-Hydroxyindole 3, optimized derivatives of trisubstituted hydroxylactam, piperidine-dione compounds described by Genentech, Inc. in WO 2015/140133, WO 2015/142903, US20200165233A1, the inhibitors described in U.S. Pat. Nos. 5,853,742 and 6,124,498, as well as those described in International Patent Application Publication No. WO 98/36774, all of which are hereby incorporated by reference.

These molecules are used in treating cancer and there is some evidence of their dampening M2 phenotypes. Indeed, lactate is thought to activate M2-like gene expression (D. Zhang et al., Metabolic regulation of gene expression by histone lactylation. Nature 574, 575-580 (2019)). The present disclosure contemplates approaches for targeting LDHA in TAMs to block their immunosuppressive M2-like phenotype to treat cancer.

The formulas of GSK2837808A and (R)-GNE-140 are represented by Formulas I and II, respectively, below.

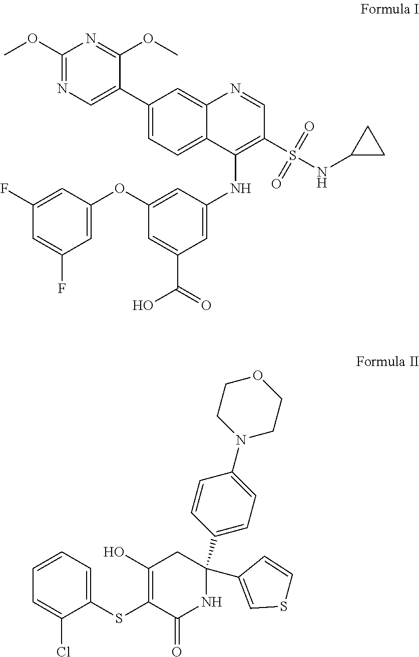 [SMILES: COc1ncc(-c2ccc3c(Nc4cc(Oc5cc(F)cc(F)c5)cc(C(=O)O)c4)c(S(=O)(=O)NC4CC4)cnc3c2)c(OC)n1 ; O=C1N[C@](c2ccc(N3CCOCC3)cc2)(c2ccsc2)CC(O)=C1Sc1ccccc1Cl]

Neoantigens. Neoantigens are peptides that are entirely absent from the normal human genome. These neo-epitopes can be created by tumor-specific DNA alterations that result in the formation of novel protein sequences. For virus-associated tumors, such as cervical cancer and a subset of head and neck cancers, neoantigens can be derived from viral open reading frames. Because they are not associated with healthy cells, neoantigens serve as an attractive target for cancer therapies, including vaccines and therapeutic approaches that selectively enhance T cell reactivity against this class of antigens. Examples of neoantigens can include the R24C mutant of CDK4, the R24L mutant of CDK4, KRAS mutated at codon 12, mutated p53, the V599E mutant of BRAF, and the R132H mutant of IDH1. The present disclosure also contemplates neoantigens known to be associated with particular cancers. Examples of neoantigens associated with glioblastoma include, but are not limited to, the EGFR (epidermal growth factor receptor) mutant (EGFRvIII), and the IDH1 (isocitrate dehydrogenase 1) mutant. Examples of neoantigens associated with ovarian cancers include, but are not limited to, the MUC-1 mutant, the TACSTD2 (tumor associated calcium signal transducer 2) mutant, the CD318 mutant, the CD 104 mutant, the N-cadherin, or the EpCAM (epithelial cell adhesion molecule) mutant. Examples of neoantigens associated with pancreatic cancers include, but are not limited to, the HSP70 mutant, the mHSP70 mutant, the MUC-1 mutant, the TACSTD2 mutant, the CEA (carcinoembryonic antigen) mutant, the CD 104 mutant, the CD318 mutant, the N-cadherin mutant, or the EpCAM1 mutant. Examples of neoantigens associated with lung cancers include, but are not limited to, mutants of EGFR, KRAS, HER2, ALK, ROS1, MET, BRAF, RET or of a member of the NTRK family. Examples of neoantigen associated with melanoma cancer cell include, but are not limited to, the melanocyte differentiation antigens, oncofetal antigens, tumor specific antigens, SEREX antigens or a combination thereof. Examples of melanocyte differentiation antigens, include but are not limited to tyrosinase, gp75, gp100, MART 1 or TRP-2. Examples of oncofetal antigens include antigens in the MAGE family (MAGE-A1, MAGE-A4), BAGE family, GAGE family or NY-ESO1. Examples of tumor-specific antigens include CDK4 and 13-catenin. Examples of SEREX antigens include D-1 and SSX-2.

LXR agonists. The liver X receptors (LXRs) are nuclear receptors whose endogenous ligands are oxysterols. LXRs are thought to function as sensors of excessive accumulation of intracellular oxysterols. These molecules can decrease pro-inflammatory genes that are targeted by NFκB and affect adipogenesis. Contemplated LXR agonists include GW3965 and T0901317/RGX10411 and their analogs. GW3965 (“GW”) and T0901317 (“TO”) have been widely utilized as non-steroidal chemical tools to explore the evolving biology of the LXRs. These compounds have been used to establish that pharmacological activation of LXRs can have therapeutic effects in atherosclerosis, type 2 diabetes, and diseases with an inflammatory component. Other non-limiting examples of LXR agonists include endogenous ligands such as oxysterols (e.g., 22(R)-hydroxycholesterol, 24(S)-hydroxycholesterol, 27-hydroxycholesterol and cholestenoic acid), synthetic agonists such as acetyl-podocarpic dimer, hypocholamide, and N,N-dimethyl-30-hydroxy-cholenamide (DMEICA).

GW and T0 are represented by Formulas III and IV, respectively, below.

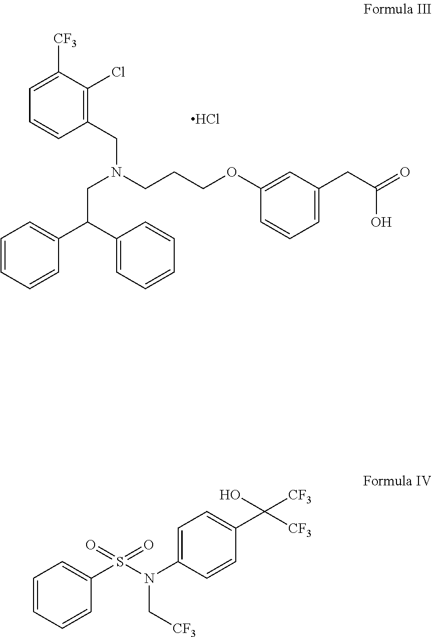 [SMILES: O=C(O)Cc1cccc(OCCCN(Cc2cccc(C(F)(F)F)c2Cl)CC(c2ccccc2)c2ccccc2)c1 ; Cl ; O=S(=O)(c1ccccc1)N(CC(F)(F)F)c1ccc(C(O)(C(F)(F)F)C(F)(F)F)cc1]

BTK inhibitors. Bruton's tyrosine kinase (BTK) is a non-receptor tyrosine kinase required for B lymphocyte development, differentiation, and signaling. BTK is highly expressed in B cell malignancies, such as chronic lymphocytic leukaemia (CLL), mantle cell lymphoma, and multiple myeloma, and the protein plays a variety of roles in maintaining and advancing malignancies. BTK is also highly expressed in monocytes and macrophages, and the latter is the key cell type that drives the development of insulin resistance which can lead to type-2 diabetes and microvascular disease. BTK inhibitors are a first-line treatment in CLL, and it is further contemplated that they can be used for treating or preventing metabolic diseases, such as obesity, insulin resistance, hyperlipidemia, hypertriglyceridemia, and type-2 diabetes and related diseases, such as microvascular disease (e.g., diabetic nephropathy). These drugs can also affect macrophages in Mycobacterium tuberculosis. Contemplated BTK inhibitors include ibrutinib, acalabrutinib (ACP-196), zanubrutinib, evobrutinib, ABBV-105 (elsubrutinib), ONO-4059/GS-4059, spebrutinib (AVL-292/CC-292), HM71224, M7583, ARQ-531, BMS-986142, dasatinib, ibrutinib, GDC-0853, PRN-1008, SNS-062, ONO-4059, BGB-3111, ML-319, MSC-2364447, RDX-022, X-022, AC-058, RG-7845, spebrutinib, TAS-5315, TP-0158, TP-4207, HM-71224, KBP-7536, M-2951, TAK-020, AC-0025, and the compounds disclosed in U.S. Patent Application Publication No. US2014/0330015 (Ono Pharmaceutical), U.S. Patent Application Publication No. US2013/0079327 (Ono Pharmaceutical), and U.S. Patent Application Publication No. US2013/0217880 (Ono Pharmaceutical), all of which are hereby incorporated by reference.

Ibrutinib is represented by Formula V below.

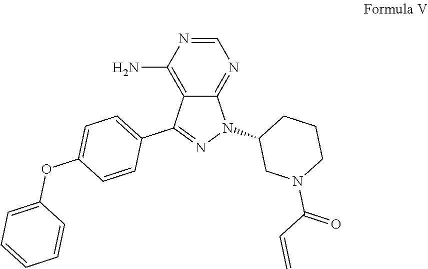 [SMILES: C=CC(=O)N1CCC[C@@H](n2nc(-c3ccc(Oc4ccccc4)cc3)c3c(N)ncnc32)C1]

SYK inhibitors. Spleen tyrosine kinase (SYK) is a non-receptor cytoplasmic enzyme that is primarily expressed in cells of hematopoietic lineage. The protein plays an important role in signal transduction in a variety of cell types. SYK has also been determined to be a mediator of formation and function of adipose tissue. Contemplated SYK inhibitors include fostamatinib (R788), entospletinib (GS-9973), cerdulatinib (PRT062070), nilvadipine, and TAK-659. Additional examples of Syk inhibitors include, without limitation, NVP-QAB205; purine-2-benzamine derivatives such as those described in U.S. Pat. No. 6,589,950, hereby incorporated by reference; pyrimidine-5-carboxamide derivatives such as those described in International Publication No. WO 99/31073, hereby incorporated by reference herein; 1,6-naphthyridine derivatives such as those described in U.S. Patent Application Publication No. US2003/0229090, hereby incorporated by reference herein; BAY 61-3606; piceatannol; 3,4-dimethyl-10-(3-aminopropyl)-9-acridone oxalate); and combinations thereof.

Therapeutic agents contemplated herein include all the categories and specific examples of compositions disclosed herein.

### Labeling Module

The DNA-based nanodevices can include one or more labels. Nucleic acid molecules can be labeled by incorporating moieties detectable by one or more means including, but not limited to, spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

Labels are chemical or biochemical moieties useful for labeling a nucleic acid. Labels include, for example, fluorescent agents, chemiluminescent agents, chromogenic agents, quenching agents, radionucleotides, enzymes, substrates, cofactors, inhibitors, nanoparticles, magnetic particles, and other moieties known in the art. Labels are capable of generating a measurable signal and can be covalently or noncovalently joined to an oligonucleotide or nucleotide and/or to a therapeutic module.

In some embodiments, the nucleic acid molecules can be labeled with a fluorescent dye or a fluorophore, which are chemical groups that can be excited by light to emit fluorescence. Some fluorophores can be excited by light to emit phosphorescence. Dyes can include acceptor dyes that are capable of quenching a fluorescent signal from a fluorescent donor dye. Dyes that can be used in the disclosed methods include, but are not limited to, the following dyes: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor® 350; Alexa Fluor® 430; Alexa Fluor® 488; Alexa Fluor® 532; Alexa Fluor® 546; Alexa Fluor® 568; Alexa Fluor® 594; Alexa Fluor® 633; Alexa Fluor® 647; Alexa Fluor® 660; Alexa Fluor® 680; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.18; Cy3.5™; Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PYMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores can include derivatives that have been modified to facilitate conjugation to another reactive molecule. As such, fluorescent dyes or fluorophores can include amine-reactive derivatives such as isothiocyanate derivatives and/or succinimidyl ester derivatives of the fluorophore.

In some embodiments, the labeling module comprises one or more contrast agents, such as magnetic particles. In some embodiments, the magnetic particles comprise iron oxide, iron platinum, manganese, and/or gadolinium. In some embodiments, the magnetic particles comprise gadolinium. In some embodiments, the labeling module comprises both one or more magnetic particles and one or more fluorescent dyes or fluorophores.

The labels can be conjugated to the nucleic acid molecules directly or indirectly by a variety of techniques. Depending upon the precise type of label used, the label can be located at the 5′ or 3′ end of the oligonucleotide, located internally in the oligonucleotide's nucleotide sequence, or attached to spacer arms extending from the oligonucleotide and having various sizes and compositions to facilitate signal interactions. Using commercially available phosphoramidite reagents, one can produce nucleic acid molecules containing functional groups (e.g., thiols or primary amines) at either terminus, for example, by coupling of a phosphoramidite dye to the 5′ hydroxyl of the 5′ base by the formation of a phosphate bond, or internally, via an appropriately protected phosphoramidite.

Nucleic acid molecules can also incorporate functionalizing reagents having one or more sulfhydryl, amino or hydroxyl moieties into the nucleic acid sequence. For example, a 5′ phosphate group can be incorporated as a radioisotope by using polynucleotide kinase and [γ32P]ATP to provide a reporter group. Biotin can be added to the 5′ end by reacting an aminothymidine residue, introduced during synthesis, with an N-hydroxysuccinimide ester of biotin. Labels at the 3′ terminus, for example, can employ polynucleotide terminal transferase to add the desired moiety, such as for example, cordycepin, 35S-dATP, and biotinylated dUTP.

Oligonucleotide derivatives are also available as labels. For example, etheno-dA and etheno-A are known fluorescent adenine nucleotides which can be incorporated into a reporter. Similarly, etheno-dC is another analog that can be used in reporter synthesis. The reporters containing such nucleotide derivatives can be hydrolyzed to release much more strongly fluorescent mononucleotides by the polymerase's 5′ to 3′ nuclease activity as nucleic acid polymerase extends a primer during PCR.

The present disclosure contemplates labeling mechanisms used with targeting. In some embodiments, fluorophore labelled DNA probes are used. In some embodiments, magnetic labelled DNA probes are used. In some embodiments, both fluorophore labels and magnetic labels are conjugated to a single nucleic acid molecule.

### Therapeutic Compositions

Therapeutic compositions contemplated herein can include one or more DNA-based nanodevices having one or more therapeutic modules and/or one or more targeting modules. In some embodiments, the therapeutic module comprises a cysteine protease inhibitor. In some embodiments, the therapeutic module comprises an LDHA inhibitor. In some embodiments, the LDHA inhibitor is (R)-GNE-140. In some embodiments, the therapeutic module comprises a BTK inhibitor. In some embodiments, the BTK inhibitor is ibrutinib.

In some embodiments, a therapeutic composition can include a pharmaceutically acceptable carrier, solvent, adjuvant, diluent, or any combination thereof. The exact nature of the carrier, solvent, adjuvant, or diluent will depend upon the desired use for the composition and can range, for example, from being suitable or acceptable for veterinary uses to being suitable or acceptable for human use.

The therapeutic compositions described herein can be provided and/or administered singly, as mixtures of one or more DNA-based nanodevices, or in a mixture or combination with other therapeutic agents useful for treating diseases, such as cancer and/or associated symptoms or other diseases. The therapeutic compositions can be administered in the form of the therapeutic compositions per se, or as pharmaceutical compositions comprising a therapeutic composition.

The therapeutic compositions of the present disclosure can be delivered through a variety of delivery methods. Delivery methodologies contemplated for delivery include, for example, the use of nanoparticles, liposomes, glucan shell microparticles, and oligopeptide complexes.

Therapeutic compositions and pharmaceutical compositions as described herein and any secondary therapeutic agents can be formulated as separate compositions that are given simultaneously or sequentially, or as a single composition. In certain embodiments, a secondary therapeutic agent can be administered in an amount below its established half maximal effective concentration (EC₅₀). For example, the secondary therapeutic agent can be administered in an amount less than 1% of, e.g., less than 10%, or less than 25%, or less than 50%, or less than 75%, or even less than 90% of the EC₅₀. In certain embodiments, the therapeutic composition can be administered in an amount below its established EC₅₀. For example, the therapeutic composition can be administered in an amount less than 1% of, e.g., less than 10%, or less than 25%, or less than 50%, or less than 75%, or even less than 90% of the EC₅₀. In certain embodiments, both a therapeutic composition as described above and a secondary therapeutic agent can be independently provided and/or administered in an amount below their respective established EC₅₀.

In certain embodiments, the therapeutic compositions of the present disclosure include one or more secondary therapeutic agents. In certain embodiments, the composition can include one or more anticancer therapeutic agents that may or may not be associated with a targeting module. Examples of anticancer agents include, but are not limited to, daunorubicin, vincristine, epirubicin, idarubicin, valrubicin, mitoxantrone, paclitaxel, docetaxel, cisplatin, camptothecin, irinotecan, 5-fluorouracil, methotrexate, dexamethasone, cyclophosphamide, etc. In some embodiments, the secondary therapeutic agent is delivered in metronomic doses. In some embodiments, the secondary therapeutic agent increases dead cell-associated antigens. In some embodiments, the secondary therapeutic agent is cyclophosphamide. In some embodiments, the cyclophosphamide is administered at a low dose. In some embodiments, the dosage and administration pattern is as follows: 50 mg/kg/intraperitoneal injection of cyclophosphamide every other day for three doses, followed by a week rest and another three doses every other day.

Further examples of secondary therapeutic agents include immune checkpoint inhibitors. These agents can include any compositions that inhibit checkpoint proteins such as PD1, CD28, CTLA-4, PD-L1, CD47, LAG-3, TIM-3, TIGIT, VISTA, and B7-H3. The agents can include antibodies that target these proteins (for example, anti-PD-L1 and anti-CD47 antibodies).

Pharmaceutical compositions can take a form suitable (can be formulated) for virtually any mode of administration, including, for example, injection, transdermal, oral, topical, ocular, buccal, systemic, nasal, rectal, vaginal, etc., or a form suitable for administration by inhalation or insufflation. Compositions that can be delivered (e.g., are formulated to be administered) intravenously, intratumorally, intraperitoneally, and/or intratracheally are also contemplated herein.

In some embodiments, a therapeutic composition of the present disclosure is included in a pharmaceutical composition having at least one pharmaceutically acceptable carrier, solvent, adjuvant, or diluent.

The term “pharmaceutical composition” is used in its widest sense, encompassing all pharmaceutically applicable compositions containing at least one active substance, and optional carriers, adjuvants, constituents, etc. The term “pharmaceutical composition” also encompasses a composition comprising an active substance in the form of a derivative or pro-drug, such as a pharmaceutically acceptable salt and/or ester. The manufacture of pharmaceutical compositions for different routes of administration falls within the capabilities of a person skilled in medicinal chemistry. The exact nature of the carrier, excipient, or diluent used in a pharmaceutical composition will depend upon the desired use for the pharmaceutical composition. The pharmaceutical composition can optionally include one or more additional compounds, such as therapeutic agents or other compounds.

The compositions described herein can be administered orally, topically, parenterally, by inhalation or spray, or rectally in dosage unit formulations containing conventional non-toxic pharmaceutically acceptable carriers, adjuvants, and vehicles. The term parenteral as used herein includes percutaneous, subcutaneous, intratumoral, intravascular (e.g., intravenous), intramuscular, or intrathecal injection or infusion techniques and the like. The pharmaceutical compositions described herein can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known in the art for the manufacture of pharmaceutical compositions and such compositions can contain one or more agents selected from the group consisting of sweetening agents, flavoring agents, coloring agents and preservative agents. Tablets contain the active ingredient in admixture with non-toxic pharmaceutically acceptable excipients that are suitable for the manufacture of tablets. These excipients can be, for example, inert diluents, such as calcium carbonate, sodium carbonate, lactose, calcium phosphate, or sodium phosphate; granulating and disintegrating agents, for example, corn starch or alginic acid; binding agents, for example, starch, gelatin, or acacia, and lubricating agents, for example, magnesium stearate, stearic acid, or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be used.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain active materials in admixture with excipients suitable for the manufacture of aqueous suspensions. Such excipients are suspending agents, for example, sodium carboxymethylcellulose, methylcellulose, hydropropyl-methylcellulose, sodium alginate, polyvinylpyrrolidone, gum tragacanth, and gum acacia; dispersing or wetting agents can be a naturally-occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example, polyethylene sorbitan monooleate. The aqueous suspensions can also contain one or more preservatives, for example, ethyl or n-propyl p-hydroxybenzoate, one or more coloring agents, one or more flavoring agents, and one or more sweetening agents, such as sucrose, sucralose, or saccharin.

Formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The therapeutic compositions described herein, or pharmaceutical compositions thereof, will generally be used in an amount effective to achieve the intended result, for example in an amount effective to treat or prevent the particular disease being treated (e.g., a therapeutically effective amount). By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally can include halting or slowing the progression of the disease.

The amount of therapeutic composition administered can be based upon a variety of factors, including, for example, the particular condition being treated, the mode of administration, whether the desired benefit is prophylactic and/or therapeutic, the severity of the condition being treated and the age and weight of the patient, the genetic profile of the patient, and/or the bioavailability of the particular therapeutic composition, etc.

Determination of an effective dosage of compound(s) for a particular use and mode of administration is well within the capabilities of those skilled in the art. Effective dosages can be estimated initially from in vitro activity and metabolism assays. For example, an initial dosage of a therapeutic composition for use in animals can be formulated to achieve a circulating blood or serum concentration of the therapeutic composition that is at or above an EC₅₀ of the particular therapeutic composition as measured in an in vitro assay. Calculating dosages to achieve such circulating blood or serum concentrations taking into account the bioavailability of the particular therapeutic composition via the desired route of administration is well within the capabilities of skilled artisans. Initial dosages of therapeutic composition can also be estimated from in vivo data, such as animal models. Animal models useful for testing the efficacy of the therapeutic composition to treat or prevent the various diseases described above are well-known in the art. Animal models suitable for testing the bioavailability of the therapeutic composition are also well-known. Ordinarily skilled artisans can routinely adapt such information to determine dosages of particular therapeutic compositions suitable for human administration.

Dosage amounts can be in the range of from about 0.0001 mg/kg/day, 0.001 mg/kg/day, or 0.01 mg/kg/day to about 100 mg/kg/day, but may be higher or lower, depending upon, among other factors, the activity of the therapeutic agent, the bioavailability of the therapeutic composition, other pharmacokinetic properties, the mode of administration and various other factors, including particular diseases being treated, the site of the disease within the body, the severity of the disease, the genetic profile, age, health, sex, diet, and/or weight of the subject. Dosage amount and interval can be adjusted individually to provide levels of the therapeutic composition which are sufficient to maintain a desired therapeutic effect. For example, a therapeutic composition can be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of therapeutic compositions may not be related to plasma concentration. Skilled artisans will be able to optimize effective dosages without undue experimentation.

### Methods

The present disclosure contemplates methods of treating diseases such as cancer, obesity, insulin resistance, Type 2 diabetes, atherosclerosis, and coronary heart disease by administration of DNA-based nanodevices having therapeutically relevant therapeutic modules. The therapeutic modules can include one or more therapeutic agents.

In some embodiments of the present disclosure, methods are presented for treating cancer. In some embodiments, a method of treating cancer in a subject in need thereof comprises administering to the subject a therapeutic composition, the therapeutic composition comprising: a nucleic acid targeting module; and a cathepsin inhibitor attached to the nucleic acid targeting module, wherein the nucleic acid targeting module targets the cathepsin inhibitor to the lysosome of a tumor associated macrophage (TAM). In some embodiments, the composition is not internalized by circulating monocytes. In some embodiments, the nucleic acid targeting module preferentially targets M2-like TAMs. In some embodiments, the method comprises reducing the lysosomal degradative capacity of the TAM. In some embodiments, the method comprises increasing cancer-derived antigen presentation or cross-presentation by the TAM. In some embodiments, the method comprises increasing intratumoral activated CD8+ cytotoxic T lymphocyte (CD45+, CD3+, CD8+, CD62L−, CD44+) populations in the subject. In some embodiments, the method comprises increasing T-cell activation and proliferation. In some embodiments, the method comprises “functionalizing” CD8⁺ T cells, which refers to activating the cells to exhibit cytotoxic effector function against particular target cells. In some embodiments, the method comprises reducing tumor volume in the subject and/or slowing the growth of one or more tumors.

Any type of cancerous solid tumor is contemplated for treatment herein, whether a primary tumor or a metastasis. For example, the tumor can originate from melanoma, breast cancer, colorectal cancer, lung cancer, ovarian cancer, liver cancer, prostate cancer, kidney cancer, bladder cancer, pancreatic adenocarcinoma, pancreatic neuroendocrine cancer, osteosarcoma, or glioblastoma. In some embodiments, the cancer is breast cancer, colorectal cancer, lung cancer, ovarian cancer, liver cancer, prostate cancer, kidney cancer, bladder cancer, pancreatic adenocarcinoma, pancreatic neuroendocrine cancer, osteosarcoma, glioblastoma, or melanoma.

In some embodiments of the present disclosure, methods of administering a therapeutic agent to a subject are presented. The methods comprise providing a therapeutic construct comprising one or more therapeutic agents attached to a nucleic acid targeting module, wherein the nucleic acid targeting module targets the therapeutic agent to the lysosome of a macrophage; and administering the therapeutic construct to the subject. The therapeutic agent is released from the lysosome of the macrophage upon degradation of the nucleic acid targeting module. In some embodiments, the therapeutic agent acts on targets in the cytosol or nucleus of the macrophage. In some embodiments, the cytosolic target is LXR. In some embodiments, providing the therapeutic agent results in the activation of LXR-target genes. In some embodiments, Abcal, Abcgl, and Apoe are activated as a result of providing the therapeutic agent.

In some embodiments, methods are used to minimize side effects of therapeutic agents. In some embodiments, a method of minimizing side-effects of a therapeutic agent includes conjugating a therapeutic agent to a nucleic acid targeting module, administering the conjugated therapeutic agent to a subject in need thereof, and releasing the therapeutic agent from the lysosome of the macrophage upon degradation of the targeting module. The nucleic acid targeting module targets the therapeutic agent to the lysosome of a macrophage. The therapeutic agent is released into the cytosol, nucleus, and/or immediate extracellular microenvironment of the macrophage to minimize side-effects of the therapeutic agent. In some embodiments, the therapeutic agent for which side effects are to be minimized comprises a small molecule. In some embodiments, the therapeutic agent for which side effects are to be minimized comprises a peptide. In some embodiments, the therapeutic agent for which side effects are to be minimized is an LXR agonist. In some embodiments, the LXR agonist for which side effects are to be minimized is GW3965 or T0901317. In some embodiments, the subject has atherosclerosis. In some embodiments, the side effect that is minimized is hyperlipidemia, hyperglyceridemia, or hypertriglyceridemia.

In some embodiments, methods are used to sensitize a subject to a therapy. The methods comprise administering to a subject a therapeutic construct comprising a therapeutic agent attached to a nucleic acid targeting module, wherein the nucleic acid targeting module targets the therapeutic agent to a lysosome of a macrophage, and administering to the subject the therapy to which the subject is to be sensitized. In some embodiments, the therapy to which the subject is to be sensitized is an anti-PD-L1 therapy. In some embodiments, the anti-PD-L1 therapy is an antibody. In some embodiments, the therapeutic agent attached to the nucleic acid targeting module is E64. In some embodiments, the nucleic acid targeting module is 38 base pairs in length.

The present disclosure contemplates methods of administering a labeling module to a subject. The methods comprise providing a labeling construct comprising a labeling module attached to a nucleic acid targeting module, wherein the nucleic acid targeting module targets the labeling construct to a lysosome of a macrophage and administering the labeling construct to the subject. The present disclosure includes a method comprising administering to a subject a labeling construct comprising a labeling module attached to a nucleic acid targeting module, wherein the nucleic acid targeting module targets the labeling module to a lysosome of a macrophage. The present disclosure further contemplates methods of imaging a biological phenomenon in a subject, comprising administering to a subject a labeling construct comprising a labeling module attached to a nucleic acid targeting module, wherein the nucleic acid targeting module targets the labeling module to a lysosome of a macrophage and detecting the labeling module. In some embodiments, the biological phenomenon is cancer or a tumor. In some embodiments, the biological phenomenon is atherosclerosis or atherosclerotic lesions. In some embodiments, the administration of the labeling construct is intravenous.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Macrophage Targeting Module

### Introduction

Experiments were conducted in an effort to determine which nucleic acid structure had the maximum efficiency of uptake.

### Methods

Various fluorescently labelled scaffolds were tested in a variety of nucleic acid configurations (Table 1).

TABLE 1
Fluorescently labelled nucleic acids tested as macrophage
targeting modules.

| | |
|---|---|
| dsDNA | SEQ ID NO: 40, SEQ ID NO: 41 |
| ssDNA | SEQ ID NO: 41 |
| ssRNA | SEQ ID NO: 43 |
| dsRNA | SEQ ID NO: 43 SEQ ID NO: 44 |
| RNA: DNA hybrid | SEQ ID NO: 45, SEQ ID NO: 46 |

BMDMs were pulsed with 50 nM of each nucleic acid scaffold for 30 min. The cells were then washed and chased for 15 min after which they were subjected to flow cytometry quantification. A nucleic acid scaffold was then selected for subsequent experiments. To test the efficiency of macrophage labeling in vivo, 25 μg of fluorophore labeled dsDNA was injected intravenously into a mouse model of triple-negative breast cancer (TNBC). To extend these results to macrophages in other tissues, 100 μg of fluorophore labeled dsDNA was injected intratracheally or intraperitoneally to label alveolar macrophages and adipose tissue macrophages, respectively.

### Results

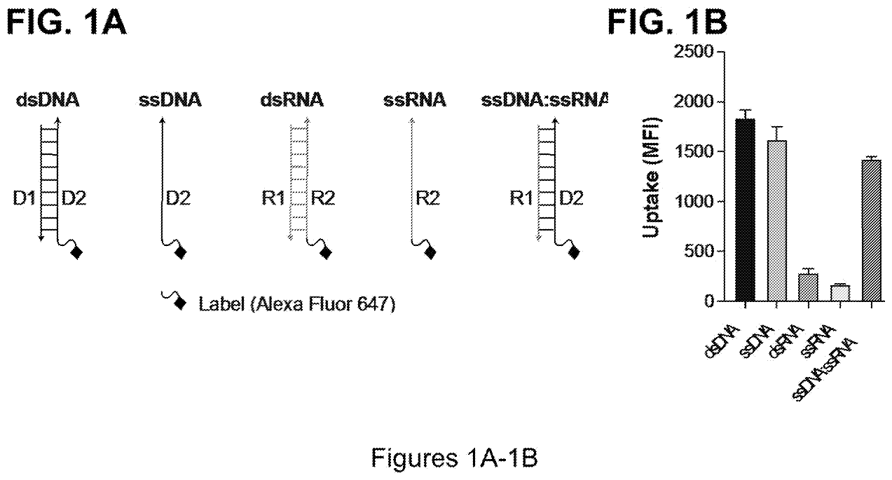

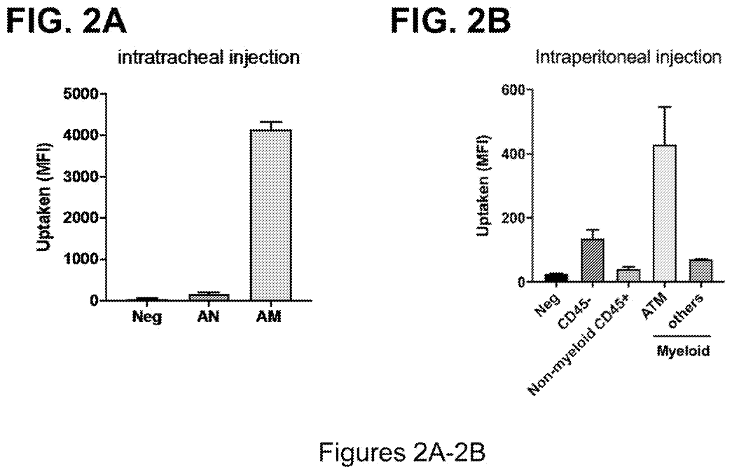

The results in BMDMs revealed maximal uptake of double stranded and single stranded DNA scaffolds (FIGS. 1A-1B). Given that dsDNA scaffold is more stable and allows for incorporation of many different modules, a dsDNA-based macrophage targeting scaffold was used for subsequent experiments. The dsDNA labeled tumor associated macrophages preferentially (>90%) over any other cell type in the tumor microenvironment in the breast cancer study. In the alveolar macrophage and adipose tissue study, the dsDNA labeled macrophages in these tissue preferentially over other cell types (>95% and 70% respectively) (FIGS. 2A-2B).

### Conclusions

These findings demonstrate that dsDNA can be used to preferentially deliver therapeutics to macrophages in many tissues. Importantly, this targeting method has been demonstrated to be independent of the sequence of the nucleic acid scaffold in Drosophila, nematodes, and macrophage cell lines.

### Example 2: Comparison of E64-DNA Uptake in Blood Versus Tumor after Intravenous Delivery

### Introduction

The experiments conducted in Example 1 led to an inquiry of whether uptake by blood cells (in addition to tumor cells) occurred after intravenous (i.v.) delivery of E64-DNA.

### Methods

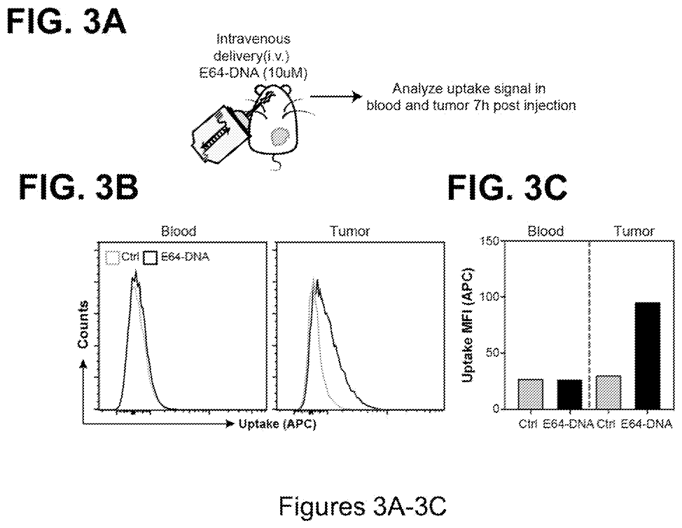

E64-DNA (25 μg) was injected intravenously into E0771 tumor-bearing mice. 7h post injection, blood was collected into EDTA coated tubes and treated with red blood cell lysis buffer to obtain blood cells, and tumors were isolated and digested to obtain tumor cells. E64-DNA uptake by blood cells and tumor cells was analyzed by flow cytometry (FIG. 3A).

### Results

Notably, there was no signal in the blood cells, indicating that E64-DNA was not internalized by circulating monocytes, T cells, etc. (FIGS. 3B-3C). In contrast, a strong signal in tumors was observed, indicating the uptake of E64-DNA by tumor cells.

### Conclusions

This property of E64-DNA distinguishes it from other macrophage delivery platforms (e.g., liposomes) that are substantially internalized by monocytes in blood and are therefore reliant (in part) on monocyte infiltration into target tissues to achieve drug delivery. The DNA-based nanodevices of the present disclosure have a specific targeting mechanism and do not rely on random infiltration into blood cells.

### Example 3: Delivery of DNA-Derivatized LXR Agonist for Treating Atherosclerosis

### Summary

State of the Art: The liver X receptor (LXR) pathway induces the expression of numerous genes involved in lipid metabolism, which protect macrophages from cholesterol accumulation and attenuate atherosclerosis.

The problem: Although LXR agonists have enormous potential as a therapy for coronary heart disease, they suffer from one important problem: they also stimulate lipid metabolism genes in hepatocytes. This induces hypertriglyceridemia in mice and eliminates their protective action in macrophages.

Approach: By derivatizing LXR agonists to nucleic targeting modules, they can be targeted specifically to macrophages (and not to hepatocytes). This approach can be advantageous because it would maintain the beneficial effects of LXR agonists in macrophages and eliminate their effects on hepatocytes, which would eliminate the unwanted side effect of hypertriglyceridemia. One uncertainty was whether or not DNA-derivatization would maintain agonist ability to induce LXR target genes in macrophages, seeing as LXR is a cytosolic protein and the targeting mechanism targets therapeutics to lysosomes.

Findings: The DNA-derivatized agonists induced LXR target genes.

### Introduction

Genetic studies in mice demonstrate that activating the LXR pathway in macrophages promotes cholesterol efflux and reduces atherosclerosis. These effects are driven by the ability of the LXR pathway to activate the expression of genes involved in lipid metabolism in macrophages. For this reason, LXR agonists have potential for treating atherosclerosis-associated diseases, such as coronary heart disease. However, LXR agonists have a key flaw: they also activate genes involved in lipid metabolism in hepatocytes. When this occurs in vivo, it leads to hypertriglyceridemia, which mitigates the beneficial action of LXR agonists on macrophages. It was reasoned that complexing LXR agonists with nucleic acid targeting modules might selectively target the drugs to macrophages to preserve their positive therapeutic actions and sequester the LXR agonists from hepatocytes to eliminate their negative side-effects.

Challenging this possibility is the fact that LXR is a cytosolic protein and that the DNA delivery platform is targeted to the lysosome of macrophages. However, because LXR agonists are small molecules, there is no concern with proteolytic destruction in the lysosome. Yet, it was unclear if the LXR agonists would be able to reach the cytosol to exert a therapeutic effect. In this example, it was sought to determine if LXR agonists complexed to DNA would have a similar capability to induce lipid metabolism genes in macrophages.

### Methods

Two different LXR agonists, T0901317 and GW3965 (TO and GW, respectively), were complexed onto DNA targeting modules. Bone marrow-derived macrophages were treated with vehicle, free DNA, DNA-agonist, and free agonist for 24 hr and monitored for effects on three known LXR target genes: Apoe, Abcal, and Abcgl. Several controls were incorporated. The free DNA treatment was included as a negative control to ensure that changes in gene expression were not due to the DNA targeting moiety. The free agonist treatment was included as a positive control to ensure that the agonist was of high quality, and to compare the efficacy of the DNA-agonist to agonist only.

### Results

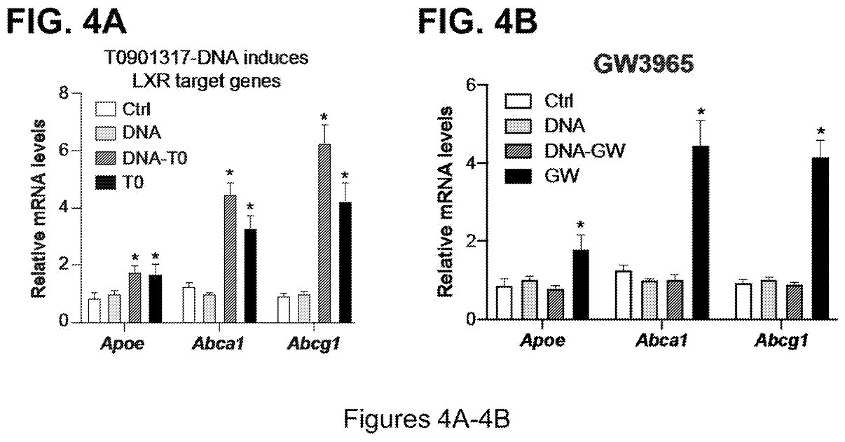

Complexing TO, but not GW, preserved its ability to target LXR in macrophages with respect to activating LXR-target genes Abcal, Abcgl, and Apoe (FIGS. 4A-4B). Results showed that T0901317-DNA could significantly induce Apoe, Abcal, and Abcgl expression in macrophages (FIG. 4A). The efficacy of T0901317-DNA was comparable to free T0901317 and not due to the DNA moiety. Unlike T0901317-DNA, GW3965-DNA was unable to induce LXR target genes, in contrast to free GW3965 (FIG. 4B). Possible explanations for the selective efficacy of T0901317-DNA include: 1) Lack of interference of remaining DNA component following DNA cleavage in the lysosome; 2) Differential ability to traffic out of the lysosome following uptake; or 3) Low concentration of agonist (GW3965).

More generally, these studies provide proof of concept that the DNA platform is able to deliver drugs not only to hit lysosomal targets (i.e., cathepsins) but also to hit macrophage cytosolic targets (i.e., LXR).

### Example 4: Additional Methods of Targeting to Macrophages

### Introduction

In cases in which the efficiency of macrophage targeting needs to be improved or a specific set of macrophages need to be labeled, further methods are developed.

### Methods

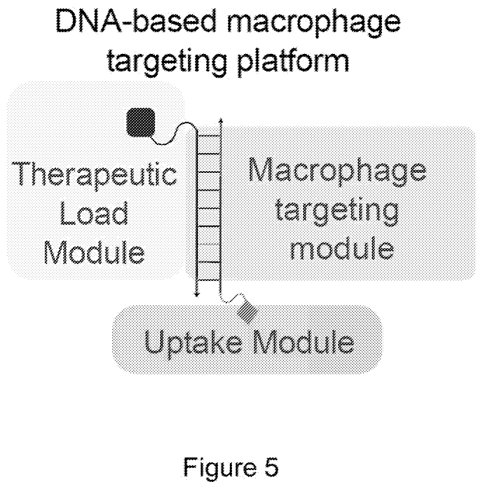

Known ligands specific to receptors on macrophages or macrophage subsets are attached to the DNA scaffold (FIG. 5). When the DNA-ligand conjugate binds to the receptor, the DNA device is endocytosed into the macrophage. Another method of targeting macrophages or macrophage subsets is by attaching aptamers (oligonucleotide or peptide molecules that bind to a specific target molecule) which have been generated against plasma membrane proteins of specific macrophage subsets.

### Example 5: Intravenous Delivery of a DNA-Derivatized Lysosomal Cysteine Protease Inhibitor

### Introduction

Activating CD8⁺ T cells by antigen cross-presentation is remarkably effective at eliminating tumors. Although this function is traditionally attributed to dendritic cells, tumor-associated macrophages (TAMs) can also cross-present antigens. TAMs are the most abundant tumor-infiltrating leukocyte. Yet TAMs have not been leveraged to activate CD8⁺ T cells because mechanisms that modulate their ability to cross-present antigens are incompletely understood. Here it is shown that TAMs harbor hyperactive cysteine protease activity in their lysosomes which impedes antigen cross-presentation, thereby preventing CD8⁺ T cell activation. A DNA nanodevice (E64-DNA) targeted to lysosomes of TAMs in mice was developed. E64-DNA inhibits the population of cysteine proteases present specifically inside lysosomes of TAMs, improves their ability to cross-present antigens, and attenuates tumor growth via CD8⁺ T cells. When combined with cyclophosphamide, E64-DNA showed sustained tumor regression in a triple-negative-breast-cancer model. These studies demonstrate that DNA nanodevices can be targeted with organelle-level precision to reprogram macrophages and achieve immunomodulation in vivo.

Tumor-associated macrophages (TAMs) are the most prevalent immune cell in the tumor microenvironment (Cassetta, L. & Pollard, J. W. Targeting macrophages: therapeutic approaches in cancer. Nat. Rev. Drug Discov. 17, 887-904 (2018)). TAMs predominantly adopt an anti-inflammatory M2-like phenotype which overexpresses growth factors (e.g. VEGFA) that promote angiogenesis, proteases (e.g. MMPs) that facilitate metastasis, and inhibitory molecules (e.g. ARG1, IL10, PD-L1) that suppress the adaptive immune response (Cassetta et al. 2018; Noy, R. & Pollard, J. W. Tumor-associated macrophages: from mechanisms to therapy. Immunity 41, 49-61 (2014); Mantovani, A., Marchesi, F., Malesci, A., Laghi, L. & Allavena, P. Tumour-associated macrophages as treatment targets in oncology. Nat. Rev. Clin. Oncol. 14, 399-416 (2017)). Depleting TAMs attenuates tumor growth and metastasis (Poh, A. R. & Ernst, M. Targeting macrophages in cancer: from bench to bedside. Front. Oncol. 8, 49 (2018); Cotechini, T., Medler, T. R. & Coussens, L. M. Myeloid cells as targets for therapy in solid tumors. Cancer J. 21, 343-350 (2015)), and high TAM abundance correlates with poor patient survival across many cancer types (Mantovani et al.; Gentles, A. J. et al. The prognostic landscape of genes and infiltrating immune cells across human cancers. Nat. Med. 21, 938-945 (2015); Takeya, M. & Komohara, Y. Role of tumor-associated macrophages in human malignancies: friend or foe? Pathol Int 66, 491-505 (2016)). Therefore, M2-like TAMs are an emerging target for anti-cancer therapy development (Cassetta et al. 2018; Mantovani et al.; Poh et al.; Vitale, I., Manic, G., Coussens, L. M., Kroemer, G. & Galluzzi, L. Macrophages and metabolism in the tumor microenvironment. Cell Metab. 30, 36-50 (2019); DeNardo, D. G. & Ruffell, B. Macrophages as regulators of tumour immunity and immunotherapy. Nat. Rev. Immunol. 19, 369-382 (2019)).

TAM phenotype can be modulated by environmental cues in the tumor microenvironment (Poh et al.). During early stages of tumor development, TAMs acquire a pro-inflammatory M1-like phenotype that opposes tumorigenesis by killing cancer cells and secreting immune-activating cytokines (Mantovani et al.; Singhal, S. et al. Human tumor-associated monocytes/macrophages and their regulation of T cell responses in early-stage lung cancer. Sci. Transl. Med. 11, (2019)). TAMs isolated from early human lung tumors cross-present antigens to activate CD8⁺ T cells (Singhal et al.).

CD8⁺ T cell activation via antigen cross-presentation effectively eliminates tumors (Fehres, C. M., Unger, W. W. J., Garcia-Vallejo, J. J. & van Kooyk, Y. Understanding the biology of antigen cross-presentation for the design of vaccines against cancer. Front. Immunol. 5, 149 (2014); Kurts, C., Robinson, B. W. S. & Knolle, P. A. Cross-priming in health and disease. Nat. Rev. Immunol. 10, 403-414 (2010)). Here, antigen-presenting cells acquire tumor antigens, displaying them on MHC class I to activate CD8⁺ T cells. Although this function is traditionally ascribed to dendritic cells (DCs) (Joffre, 0. P., Segura, E., Savina, A. & Amigorena, S. Cross-presentation by dendritic cells. Nat. Rev. Immunol. 12, 557-569 (2012)), TAMs and macrophages can also cross-present antigens, albeit less efficiently (Singhal et al.; Cruz-Leal, Y. et al. The Vacuolar Pathway in Macrophages Plays a Major Role in Antigen Cross-Presentation Induced by the Pore-Forming Protein Sticholysin II Encapsulated Into Liposomes. Front. Immunol. 9, 2473 (2018); Embgenbroich, M. & Burgdorf, S. Current Concepts of Antigen Cross-Presentation. Front. Immunol. 9, 1643 (2018); Shen, L., Sigal, L. J., Boes, M. & Rock, K. L. Important role of cathepsin S in generating peptides for TAP-independent MHC class I crosspresentation in vivo. Immunity 21, 155-165 (2004)). Because TAMs are more abundant and phagocytic than DCs in tumors (Cassetta et al. 2018; Noy et al.), experiments were attempted to harness them to directly activate CD8⁺ T cells to attack tumors. However, such an approach is impeded by an incomplete understanding of mechanisms limiting antigen cross-presentation by M2-like TAMs, as well as technologies to target therapeutics to TAMs in vivo.

Using unbiased proteomics, it was found that M2-like TAMs have elevated lysosomal cysteine protease activity which hampers antigen cross-presentation and prevents CD8⁺ T cell activation. A method to chemically inhibit cysteine proteases in lysosomes of M2-like TAMs was developed. DNA scaffolds have enabled targeted delivery of chemical imaging agents to lysosomes in phagocytic cells by exploiting receptor-mediated endocytosis (Surana, S., Bhat, J. M., Koushika, S. P. & Krishnan, Y. An autonomous DNA nanomachine maps spatiotemporal pH changes in a multicellular living organism. Nat. Commun. 2, 340 (2011); Chakraborty, K., Leung, K. & Krishnan, Y. High lumenal chloride in the lysosome is critical for lysosome function. Elife 6, e28862 (2017); Narayanaswamy, N. et al. A pH-correctable, DNA-based fluorescent reporter for organellar calcium. Nat. Methods 16, 95-102 (2019); Leung, K., Chakraborty, K., Saminathan, A. & Krishnan, Y. A DNA nanomachine chemically resolves lysosomes in live cells. Nat. Nanotechnol. 14, 176-183 (2019); Dan, K., Veetil, A. T., Chakraborty, K. & Krishnan, Y. DNA nanodevices map enzymatic activity in organelles. Nat. Nanotechnol. 14, 252-259 (2019); Veetil, A. T. et al. DNA-based fluorescent probes of NOS2 activity in live brains. Proc. Natl. Acad. Sci. USA 117, 14694-14702 (2020)).

A DNA nanodevice (E64-DNA) displaying a cysteine protease inhibitor (E64) was created. E64-DNA preferentially localizes to TAMs via scavenger receptor-mediated endocytosis and traffics to lysosomes. By inhibiting cysteine protease activity therein, E64-DNA improves antigen cross-presentation in TAMs, which activates CD8⁺ T cells to oppose tumorigenesis. These studies identified elevated lysosomal cysteine protease activity in M2-like TAMs as an important, yet targetable, innate immune blockade in anti-tumor immunity.

### Methods

Regulatory. Animal studies were approved by the Institutional Animal Care and Use Committee (ACUP #72209, #72504) at the University of Chicago. Cancer cell lines were approved by the Institutional Biosafety Committee (IBC #1503). Human studies were approved by the Institutional Review Boards at the University of Chicago (IRB160321) and Northwestern University (NU-IRB #STU00023488).

Mice. 6-7-week-old C57BL/6 female mice, LysMcre knock in mice, OT-1, OT-2, Scarb1−/−, Cd36−/− and Msr1−/− mice were purchased from The Jackson Laboratory®. Tfeb^{fl/fl} mice were a gift from Dr. Andrea Ballabio. pMel and TRP1 mice were a gift from Dr. Melody Swartz, University of Chicago. Myeloid cell specific Tfeb−/− mice (mTfeb−/−) and their littermate controls (fl/fl) were generated by crossing Tfeb^{fl/fl} mice with LysMcre+/− mice. Mouse genotype was confirmed by PCR (Table 2).

TABLE 2
Primers for PCR analysis.

| Mouse gene | Forward primer | Reverse primer |
|---|---|---|
| 18s | GCCGCTAGAGGTGAAATTCTT | CGTCTTCGAACCTCCGACT (SEQ ID |
| | (SEQ ID NO: 47) | NO: 48) |
| Ctsb | CTGCGCGGGTATTAGGAGT (SEQ | CAGGCAAGAAAGAAGGATCAAG (SEQ |
| | ID NO: 4) | ID NO: 5) |
| Cstl | AGACCGGCAAACTGATCTCA | ATCCACGAACCCTGTGTCAT (SEQ ID |
| | (SEQ ID NO: 6) | NO: 7) |
| Ctsz | GGCCAGACTTGCTACCATCC | ACACCGTTCACATTTCTCCAG (SEQ ID |
| | (SEQ ID NO: 8) | NO: 9) |
| Lipa | CTGGTGAGGAACACTCGGTC | AGCCGTGCTGAAGATACACAA (SEQ |
| | (SEQ ID NO: 10) | ID NO: 11) |
| Lgmn | ATTCCTGACGAGCAGATCATAGT | GTGCCGTTAGGTCGGTTGA (SEQ ID |
| | (SEQ ID NO: 12) | NO: 13) |
| Tnfa | CACCACGCTCTTCTGTCTACTG | GCTACAGGCTTGTCACTCGAA (SEQ ID |
| | (SEQ ID NO: 14) | NO: 15) |
| Il1b | AACTCAACTGTGAAATGCCACC | CATCAGGACAGCCCAGGTC (SEQ ID |
| | (SEQ ID NO: 16) | NO: 17) |
| Nos2 | GCTCCTCTTCCAAGGTGCTT (SEQ | TTCCATGCTAATGCGAAAGG (SEQ ID |
| | ID NO: 18) | NO: 19) |
| Arg1 | CTCCAAGCCAAAGTCCTTAGAG | AGGAGCTGTCATTAGGGACATC (SEQ |
| | (SEQ ID NO: 20) | ID NO: 21) |
| Il10 | GCTCTTACTGACTGGCATGAG | CGCAGCTCTAGGAGCATGTG (SEQ ID |
| | (SEQ ID NO: 49) | NO: 50) |
| Fizz1 | CCTGCTGGGATGACTG (SEQ ID | TGGGTTCTCCACCTCTTCAT (SEQ ID |
| | NO: 24) | NO: 25) |
| Gapdh | TGGCCTTCCGTGTTCCTAC (SEQ | GAGTTGCTGTTGAAGTCGCA (SEQ ID |
| | ID NO: 26) | NO: 27) |
| Cd11b | CCATGACCTTCCAAGAGAATGC | ACCGGCTTGTGCTGTAGTC (SEQ ID |
| | (SEQ ID NO: 28) | NO: 29) |
| Sqstm1 | GAGTAACACTCAGCCAAGCA | TTCACCTGTAGATGGGTCCA (SEQ ID |
| | (SEQ ID NO: 30) | NO: 31) |
| Map1lc3b | TTGCAGCTCAATGCTAACCA | GGCATAAACCATGTACAGGA (SEQ ID |
| | (SEQ ID NO: 32) | NO: 33) |
| Vps11 | AAAAGAGAGACGGTGGCAATC | AGCCCAGTAACGGGATAGTTG (SEQ |
| | (SEQ ID NO: 34) | ID NO: 35) |
| Uvrag | CTGACAGAAAAGGAGCGAGA | GGATGGCATTGGAGATGTGA (SEQ ID |
| | (SEQ ID NO: 36) | NO: 37) |
| Atg9b | CCATCCCACAATGATACACACC | CCTCTAGCCGTTCATAGTCCT (SEQ ID |
| | (SEQ ID NO: 38) | NO: 39) |
| Vps18 | AGTACGAGGACTCATTGTCCC | TGGGCACTTACATACCCAGAAT (SEQ |
| | (SEQ ID NO: 51) | ID NO: 52) |
| Becn1 | AGGTACCGACTTGTTCCCTA | TCCATCCTGTACGGAAGACA (SEQ ID |
| | (SEQ ID NO: 53) | NO: 54) |
| Tfeb | CAAGGAGCGGCAGAAGAAAG | GCTGCTTGTTGTCATCTCC (SEQ ID |
| | (SEQ ID NO: 55) | NO: 56) |
| Human gene | Forward primer | Reverse primer |
| 18S | CCCAACTTCTTAGAGGGACAAG | CATCTAAGGGCATCACAGACC (SEQ |
| | (SEQ ID NO: 57) | ID NO: 58) |
| CTSB | GAGCTGGTCAACTATGTCAACA | GCTCATGTCCACGTTGTAGAAGT (SEQ |
| | (SEQ ID NO: 59) | ID NO: 60) |
| CTSL | AAACTGGGAGGCTTATCTCACT | GCATAATCCATTAGGCCACCAT (SEQ |
| | (SEQ ID NO: 61) | ID NO: 62) |
| CTSZ | ACCAATGTGGGACATGCAATG | TTGCGTAGATTTCTGCCATCA (SEQ ID |
| | (SEQ ID NO: 63) | NO: 64) |
| LIPA | CCCACGTTTGCACTCATGTC (SEQ | CCCAGTCAAAGGCTTGAAACTT (SEQ |
| | ID NO: 65) | ID NO: 66) |
| LGMN | TCCGGCAAAGTCCTGAAGAG | GGCAGCAGTAGTTGCATAAACA (SEQ |
| | (SEQ ID NO: 67) | ID NO: 68) |
| TNFA | CAGCCTCTTCTCCTTCCTGAT | GCCAGAGGGCTGATTAGAGA (SEQ ID |
| | (SEQ ID NO: 69) | NO: 70) |
| IL1B | TCTGTACCTGTCCTGCGTGT (SEQ | ACTGGGCAGACTCAAATTCC (SEQ ID |
| | ID NO: 71) | NO: 72) |
| IL12 | GCGGAGCTGCTACACTCTC (SEQ | CCATGACCTCAATGGGCAGAC (SEQ |
| | ID NO: 73) | ID NO: 74) |
| NOS2 | CAGCGGGATGACTTTCCAAG | AGGCAAGATTTGGACCTGCA (SEQ ID |
| | (SEQ ID NO: 75) | NO: 76) |
| CD206 | GGCGGTGACCTCACAAGTAT | ACGAAGCCATTTGGTAAACG (SEQ ID |
| | (SEQ ID NO: 77) | NO: 78) |
| ARG1 | GGCAAGGTGATGGAAGAAAC | AGTCCGAAACAAGCCAAGGT (SEQ ID |
| | (SEQ ID NO: 79) | NO: 80) |
| IL10 | GGGAGAACCTGAAGACCCTC | ATAGAGTCGCCACCCTGATG (SEQ ID |
| | (SEQ ID NO: 81) | NO: 82) |
| MMP12 | CATGAACCGTGAGGATGTTGA | GCATGGGCTAGGATTCCACC (SEQ ID |
| | (SEQ ID NO: 83) | NO: 84) |

| Genotyping primers | Reverse primer |
|---|---|
| Tfebfl/fl Forward | GTAGAACTGAGTCAAGGCATACTGG |
| | (SEQ ID NO: 1) |
| Tfebfl/fl Reverse | GGGTCCTACCTACCACAGAGCC (SEQ |
| | ID NO: 2) |
| loxp-R | CTTCGTATAATGTATGCTATACGAAG |
| | (SEQ ID NO: 3) |

Mice were housed in the specific pathogen-free animal facility at the Gordon Center for Integrative Science building at the University of Chicago. A 12 light/12 dark cycle is used. Temperatures of 68-74° F. with 30-70% humidity are maintained. For monitoring tumor growth, mice were sacrificed once tumors reached 1000 mm 3 in size.

Cell Culture. E0771 cells were a gift from Dr. Marsha Rosner, University of Chicago; commercially available from ATCC (CRL-3461™). LLC1 cells were purchased from ATTC (CRL-1642™). B16F10 cells were a gift from Dr. Thomas Gajewski, University of Chicago, commercially available from ATCC (CRL-6475™). B16.0VA cells were a gift from Dr. Jeffrey Hubbell, University of Chicago. Cells were cultured in Dulbecco's Modified Eagles Medium (DMEM; HyClone®) containing 10% heat-inactivated FBS (Gemini Bio™-Products) and 1% penicillin/streptomycin (Gibco®).

Isolation and activation of bone marrow-derived macrophage (BMDM). BMDMs were differentiated from bone marrow stem cells with L-cell conditioned media for six days as previously described (Kratz, M. et al. Metabolic dysfunction drives a mechanistically distinct proinflammatory phenotype in adipose tissue macrophages. Cell Metab. 20, 614-625 (2014)). For M1 activation, BMDMs were treated with LPS (5 ng/mL, Sigma®) and IFNγ (12 ng/mL, R&D Systems®) for 24h. For M2 activation, BMDMs were treated with IL-4 (20 ng/mL, R&D Systems®) for 48h.

Murine adipose tissue macrophage (ATM) isolation. Adipose tissue was digested with Type 1 Collagenase (Worthington, 1 mg/mL) at 37° C. with shaking at 160 rpm for 45 min. Digested tissue was filtered through a 100 μm cell strainer, incubated in RBC lysis buffer for 5 min, and passed through a 40 μm cell strainer. ATMs were isolated using CD11b microbeads (Miltenyi Biotec®) as previously described (Kratz, M. et al. Metabolic dysfunction drives a mechanistically distinct proinflammatory phenotype in adipose tissue macrophages. Cell Metab. 20, 614-625 (2014)). Purity was assessed by flow cytometry.

Murine tumor processing. Tumors were digested with Type 4 Collagenase (Worthington, 3 mg/mL) and hyaluronidases (Sigma®, 1.5 mg/mL) at 37° C. with horizontal shaking at 200 rpm for 45 min (E0771) or 30 min (LLC1 and B16F10). Digested tumor was filtered through a 100 μm cell strainer, incubated in RBC lysis buffer for 5 min, and passed through a 40 μm cell strainer.

Tumor immune cell analyses—Cells were labeled with various antibodies (see below) and analyzed by flow cytometry.

Isolation of M1-like and M2-like TAMs—Cells were resuspended in isolation buffer (0.1% BSA/PBS, 2 mM EDTA), layered onto Ficoll-Paque™ PLUS (GE Healthcare), and centrifuged at 450×g for 30 min. Mononuclear cells were obtained by collecting the middle white layer. Enriched mononuclear cells were stained with antibodies, and M1-like and M2-like tumor-associated macrophages (TAMs) were sorted using a BD FACS Aria™ Fusion cell sorter or Ariall 4-15.

Isolation of pooled TAMs—TAMs were isolated using CD11b microbeads (Miltenyi Biotec®) according to the manufacturer's instruction, and purity was assessed by flow cytometry.

Antibodies—CD45 (47-0451), CD11b (25-0112), MHCII (11-5321), Ly6C (12-5932), CD4 (17-0041), CD8 (12-0081), CD44 (25-0441), CD69 (11-0691) from ThermoFisher Scientific®; CD3 (551163), CD62L (561917), CD11c (561241), Gr1(553129) from BD Biosciences, and Ly6G (127614), CD103 (121415), CD206 (141706), OX40 (119414), CD3 (100220), 4-1BB (106106) from BioLegend®. For staining one million cells in 100 μl volume, all antibodies were used at 1:100 dilution. Viability was assessed by calcein blue AM (BD Biosciences). Flow data were collected by BD FACSDiva™ and quantified by FlowJo® v.10.4.1.

5-Bromo-2″-Deoxyuridine (BrdU) incorporation. Tumor bearing mice were intraperitoneally injected with 50 mg/kg of BrdU (B23151, ThermoFisher Scientific®) for two consecutive days prior to sacrifice. Tumor were isolated, digested, and stained with anti-BrdU antibody (11-5071, ThermoFisher Scientific®).

Isolation and activation of human peripheral blood monocyte-derived macrophages (HMDMs). Monocytes were purified from the blood of healthy donors using CD14 microbeads (MiltenylBiotec®) and differentiated into HMDMs using human M-CSF (125 ng/mL, R&D Systems®) for 7 days as previously described (Kratz, M. et al. Metabolic dysfunction drives a mechanistically distinct proinflammatory phenotype in adipose tissue macrophages. Cell Metab. 20, 614-625 (2014)). For M1 activation, HMDMs were treated with LPS (100 ng/mL, Sigma®) and IFNγ (1 ng/mL, R&D Systems®) for 24h. For M2 activation, HMDMs were treated with IL-4 (10 ng/mL, R&D Systems®) and IL-10 (10 ng/mL), R&D Systems® for 48h.

Human breast tumor tissue processing and immune analysis. Human breast tumor tissue was cut into ˜100 mg pieces, each of which was digested in HB SS Ca²⁺/Mg²⁺ buffer containing TL (14 U/mL) and DL (28 U/mL) (Roche) and DNAse I (15 mg/mL) at 37° C. with horizontal shaking at 200 rpm for 45 min, adapted from previously described (Cassetta, L. et al. Human Tumor-Associated Macrophage and Monocyte Transcriptional Landscapes Reveal Cancer-Specific Reprogramming, Biomarkers, and Therapeutic Targets. Cancer Cell 35, 588-602.e10 (2019)). Digested tumors were filtered through a 100 μm cell strainer, incubated in RBC lysis buffer for 5 min, passed through a 40 μm cell strainer, and resultant cells were resuspended in isolation buffer (0.1% BSA/PBS, 2 mM EDTA). For DQ-OVA degradation assays, cells were incubated with DQ-ovalbumin (see below) and DQ-OVA fluorescence was quantified in CD45⁺CD11b⁺CD14⁺CD163⁺TAMs.

Antibodies—CD11b (17-0118) from ThermoFisher Scientific®; CD45 (557748), CD163 (563887), CD14 (347497), HLA-DR (560651) from BD Biosciences. CD206 (321120) from BioLegend®. For staining one million cells in 100 μl volume, all antibodies were used at 1:20 dilution except CD14 (347497) was used as 1:5 dilution. Viability was assessed by calcein blue AM (BD Biosciences). Flow cytometry data were quantified by FlowJo v.10.4.1.

Thioglycolate-elicited peritoneal macrophage isolation. Peritoneal macrophages were isolated as previously described (Reardon, C. A. et al. Obesity and Insulin Resistance Promote Atherosclerosis through an IFNγ-Regulated Macrophage Protein Network. Cell Rep. 23, 3021-3030 (2018)). Briefly, peritoneal macrophages were collected by lavaging the peritoneal cavity with PBS containing 2% endotoxin-free BSA (Sigma) 5 days after 4% thioglycolate injection (3 mL/mouse). Purity was assessed by flow cytometry.

Cytosolic and nuclear extractions. For cytosolic extraction, cell pellets were resuspended in 5× volume of cytoplasmic extraction buffer (10 mM HEPES, 10 mM KCl, 0.1 mM EDTA, 0.3% NP-40, protease inhibitors), incubated on ice for 5 min with vortexing, and centrifuged at 3500×g for 5 min at 4° C., and the supernatant was harvested. For nuclear extraction, cell pellets were washed twice with 5× volume of cytoplasmic extraction buffer without NP-40, resuspended with 1× volume of nuclear extraction buffer (20 mM HEPES, 0.4 M NaCl, 1 mM EDTA, 25% glycerol, protease inhibitors), incubated on ice for 10 min with vortexing, centrifuged at 900×g for 5 min at 4° C., and the supernatant was harvested.

Analysis of lysosome number. Macrophages were seeded on imaging dishes (Cellvis). After attachment, cells were stained with anti-LAMP1 antibody (ab24170, Abeam®, 1:250 dilution) to mark lysosomes, followed by a DyLight™ 594 secondary antibody (ab96893, Abeam®, 1:500 dilution) and DAPI (Vectashield® H-1500) for nuclear staining. Fluorescence images were acquired with a Nikon® Eclipse Ti2 microscope with the following settings: objective magnification 90×, objective numerical aperture 0.45, room temperature, emission wavelengths of 457.5 nm (DAPI), 535.0 nm (GFP), and 610 nm (RFP), Camera Nikon DS-Qi2, and NIS-Elements® Version 5.02 software. Analysis was performed using brightfield to denote the area and perimeter of the cell. LAMP1 was imaged in RFP and thresholding was set using bright spot detection. Adjacent cells were separated using a watershed function centered on the nucleus. LAMP1 signal was quantified using the number of LAMP1 signals per unit of cell area.

Analysis of lysosomal degradation by DQ-OVA. Lysosomal degradative capacity of macrophages was assessed by a DQ-OVA degradation assay (D-12053, Invitrogen®) according to the manufacturer's instruction. Briefly, 0.2 million cells were incubated with 10 μg/mL of DQ-OVA at room temperature for 15 min, washed, and incubated at 37° C. for another 15 min. DQ-OVA fluorescence was quantified by flow cytometry.

Analysis of lysosome pH. Lysosomal pH of macrophages was assessed by LysoTracker™ Red DND-99 (ThermoFisher Scientific®) according to the manufacturer's instructions. In brief, 0.5 million cells were incubated with 100 nM lysotracker at 37° C. for 1 h. Signals were quantified by flow cytometry.

Analysis of cysteine cathepsin activity by ProSense 680. Cysteine cathepsin activity of M1-like and M2-like TAMs was assessed by ProSense 680, an activity-based fluorescent imaging agent (NEV10003, PerkinElmer®) according to the manufacturer's instruction. Briefly, 1 million cells were first incubated with ProSense® 680 at a final concentration of 1 μM for 6h at 37° C., followed by other cell surface antibody staining for 15 min at room temperature (distinguishes M1-like and M2-like TAMs). Cells were washed, and fluorescence signals were quantified by flow cytometry.

Cell viability assays. TAMs were plated in complete growth media and treated with vehicle, DNA, E64, or E64-DNA (100 nM) for 72h, and cell viability was assessed by Calcein-AM (ThermoFisher Scientific®, 4 ng/mL). Fluorescence was measured at 495 nm/516 nm using a Synergy® HT Multi-Mode Microplate Reader (Biotek®) and data was obtained by Gen5 3.03 software.

Cell proliferation assay. E0771 cells were seeded in a 96 well clear bottom plate (Greiner Bio-One®) at 2000 cells/well. Once cells adhered, the plate was placed into the IncuCyte® S3 live-cell analysis system and warmed to 37° C. for 30 min prior to scanning. Each well was scanned every 4h, and the % confluency was quantified by IncuCyte® S3 plate Map Editor 2018B software.

Western blot analyses. Cells were lysed with 1% SDS containing protease and phosphatase inhibitors (Sigma), and protein was quantified with the BCA Protein Assay Kit (Pierce). Proteins (10-20 μg) were resolved on 10%, 12.5%, or 15% SDS-PAGE gels depending on the target protein, transferred to PVDF membranes (Millipore®), blocked with 5% BSA (Sigma®) in TBS/Tween-20 (0.05%) at RT for 2h, stained with primary and secondary antibodies, and visualized using the ECL detection kit (Biorad) and a LI-COR imager with Image Studio software version 2.1.10.

Antibodies—Antibodies against murine TFEB (A303-673A, Bethyl Laboratories), CTSL (af1515, R&D Systems®), CTSB (3171, CST), Tubulin (2125, CST), CTSZ (sc-376976, Santa Cruz Biotechnology), BLOC1S1 (SC-515444, Santa Cruz Biotechnology), LIPA (sc-58374, Santa Cruz Biotechnology). LMNB1 (13435, CST), IRF3 (sc-33641, Santa Cruz Biotechnology), p-IRF3 (29047, CST), p-TBK1 (5483, CST), TBK1 (3504, CST), LC3 (L7543, Sigma), p62 (nbpl-49954, Novus Biologicals), CTSE (SC-166500, Santa Cruz Biotechnology), CTSD (SC-377124, Santa Cruz Biotechnology). All antibodies were used at 1:1000 dilution.

Shotgun proteomics. Whole cell lysates from M1 and M2 BMDMs and from flow sorted M1-like and M2-like TAMs were collected in 4% sodium deoxycholate (SDC) in 10 mM Tris, 1 mM EDTA, pH 7.4 for trypsin digestion. Samples were denatured by heating at 56° C. and reduced with 5 mM dithiothreitol (DTT) for 1 h, alkylated with 15 mM iodoacetamide for 30 min at room temperature in the dark, and excess iodoacetamide was quenched with an additional 5 mM DTT. Samples were digested with trypsin (Promega, Madison, WI) at 1:20 w/w ratio overnight at 37° C. with mixing. After digestion, SDC was precipitated by addition of 1% trifluoroacetic acid and insoluble material was removed by centrifugation at 14,000×g for 10 min. Samples were then desalted by solid phase extraction using Oasis HLB 96-well μElution Plate, dried down, stored at −80° C., and reconstituted with 0.1% formic acid in 5% acetonitrile to a peptide concentration of 0.1 μg/μL for LC-MS analysis.

LC/MS analyses. Digested peptides were injected onto a trap column (40×0.1 mm, Reprosil C18, 5 μm, Dr. Maisch, Germany), desalted for 5 min at a flow of 4 μL/min, and separated on a pulled tip analytical column (300×0.075 mm, Reprosil C18, 1.9 μm, Dr. Maisch, Germany) with a 3 segment linear gradient of acetonitrile, 0.1% FA (B) in water, 0.1% FA (A) as follows: 0-2 min 1-5% B, 2-150 min 5-25% B, 150-180 min 25-35% B followed by column wash at 80% B and re-equilibration at a flow rate 0.4 μL/min (Waters™ NanoACQUITY UPLC®). Tandem MS/MS spectra were acquired on Orbitrap Fusion Lumos (Thermo Scientific) operated in data-dependent mode on charge states 2-4 with 2s cycle time, dynamic exclusion of 30s, HCD fragmentation (NCE 30%) and MS/MS acquisition in the Orbitrap. MS spectra were acquired at a resolution 120,000 and MS/MS spectra (precursor selection window 1.6 Da) at a resolution of 30,000. Peptides and proteins were identified using the Comet search engine (Eng, J. K. et al. A deeper look into Comet—implementation and features. J Am Soc Mass Spectrom 26, 1865-1874 (2015)) with PeptideProphet and ProteinProphet validation. Search criteria included a 20 ppm tolerance window for precursors and products, fixed Cys alkylation, and variable Met oxidation.

Measurement of gene expression by qRT-PCR. Cell pellets were lysed in RLT buffer, total RNA was isolated using the RNAeasy kit (Qiagen®) with on-the-column DNAse digestion (Qiagen®), converted to cDNA using reverse transcription kit (Qiagen®), and amplified using QuantiTect SYBR Green PCR Kits (Qiagen). Data was obtained by StepOne software v2.3. Primers are listed in Table 2.

In vitro antigen destruction assay. gp10025-33 (1.5 μg) was incubated with vehicle (Veh; PBS), cysteine proteases (CPs) (0.1 μg CTSB and 0.1 μg CTSL), or aspartic proteases (APs) (0.1 μg CTSD and 0.1 μg CTSE) in pH 5 sodium acetate buffer at 37° C. for 3h. Degradation was stopped by adjusting to pH 7.4 with cell culture media (dilution to 10 μg/mL). Inhibition of CPs and APs was confirmed by activity assays and diluted solution was subsequently used for antigen cross-presentation assays.

PepA-DNA in vivo experiments. PepA-DNA or DNA (25 μg) was intravenously delivered (i.v.; retro-orbital) into B16.0VA tumor-bearing mice. Tumor growth was measured over 8 days after a single injection.

Nucleic acid synthesis. Amine labeled 38-mer DNA (D1), Alexa 647 labeled complementary DNA strand (D2), RNA (R1), and Alexa 647 labeled RNA strand (R2) were obtained from IDT (Table 3).

TABLE 3
DNA nanodevice devices.

| Name | sequence |
|---|---|
| D1 | ATCAACACTGCACACCAGACAGCAAGATCCTATATATA (SEQ ID NO: |
| | 40) |
| D2 | Alexa647TATATATAGGATCTTGCTGTCTGGTGTGCAGTGTTGAT (SEQ |
| | ID NO: 41) |
| R1 | AUCAACACUGCACACCAGACAGCAAGAUCCUAUAUAUA (SEQ ID NO: |
| | 44) |
| R2 | Alexa647UAUAUAUAGGAUCUUGCUGUCUGGUGUGCAGUGUUGAU |
| | (SEQ ID NO: 43) |

HPLC-purified oligonucleotides were dissolved in Milli-Q water to make 100 μM stock solutions and quantified using an ultraviolet spectrophotometer and stored at −20° C. To prepare a DNA or RNA duplex sample (i.e. D1-D2, or R1-R2), 50 μM of each complementary strand were mixed in equimolar ratios in 20 mM sodium phosphate buffer (pH 7.2) containing 100 mM KCl. The resultant solution was heated to 90° C. for 15 min, cooled to room temperature at 5° C. per 15 min, and kept at 4° C. overnight.

E64-DNA or PepA-DNA synthesis. E64 (Selleckchem®) or Pepstatin A (PepA, GoldBio®) was conjugated to the amine labeled DNA duplex via EDC coupling. Briefly, 2 mM E64 was incubated with N-hydroxysuccinimide (NHS) and 1-ethyl-3-(−3-dimethylaminopropyl) carbodiimide hydrochloride (EDC, each 2 equivalents excess) in 10 mM IVIES buffer at pH 5.0 for 1 hour at room temperature. The solution was then added to the DNA duplex sample in two rounds and incubated for 24 hours. To remove excess E64, NHS, and EDC, the reaction mixture was passed through a 3 kDa cut-off centrifugal filter (Amicon, Millipore) and washed multiple times. E64-DNA or PepA-DNA was stored at 4° C. till further use.

E64-DNA Uptake.

E64-DNA trafficking to lysosome in vitro—TAMs were allowed to adhere to 8 well dishes, pulsed with TMR-Dextran (0.5 mg/mL) in complete medium for 1 h, washed with PBS, and cultured for 16h to allow TMR-Dextran to traffic to lysosomes. At this time, TAMs were treated with E64-DNA (100 nM) for 30 min, washed with PBS, and imaged 30 min later using a Leica SP5 confocal microscope. Images were obtained and analyzed using LAS AF Leica confocal software and ImageJ/Fiji 1.51, respectively.

E64-DNA trafficking to lysosome in vivo—E64-DNA (25 μg) was injected intratumorally (i.t.) into E0771 tumor-bearing mice. TAMs were isolated 1 h after injection, allowed to adhere to 8 well dishes, and pulsed with LysoTracker™ DND-99 (100 nM, ThermoFisher Scientific) in complete medium for 30 min. After a PBS wash, TAMs were imaged using a Leica SP5 confocal microscope. Images were obtained and analyzed using LAS AF Leica confocal software and ImageJ/Fiji 1.51 respectively.

E64-DNA uptake by M2 BMDMs in vitro—E64-DNA (100 nM) or other types of nucleic acids (D1-D2, D2, R1-R2, R2) was incubated with 0.2 million M2-activated BMDMs from wt, Scarb1−/−, Msr1−/−, or Cd36−/− mice for 30 min, washed with PBS, and uptake was assessed by flow cytometry.

For the in vitro M1 and M2 macrophage E64-DNA uptake competition assay—M2 BMDMs were labeled with Hoechst dye 33342 (2 μg/mL, ThermoFisher Scientific) in a tube for and washed with PBS twice. M1 and M2 BMDMs were co-incubated at a 1:1 ratio (0.2 million cells total) with E64-DNA (100 nM) for 15 min, washed with PBS, and E64-DNA uptake was assessed by flow cytometry.

E64-DNA uptake in vivo—E64-DNA (25 μg) was injected intratumorally (i.t.) or intravenously (i.v.) by the retro-orbital route into E0771 tumor-bearing mice. Tumors were isolated 7h after injection, digested, and E64-DNA uptake was assessed by flow cytometry.

dsDNA serum stability. 10 μM dsDNA was added to 100% mouse serum obtained from 8-week-old C57/BL6 mice and incubated for various time points (0-24h) at 37° C. DNA degradation was assessed using 18% polyacrylamide gels stained with SYBR™ Gold (ThermoFisher Scientific). Image of DNA electrophoresis gel was obtained by GeneFlash Syngene Bio Imaging machine.

CD8⁺ T cell and CD4⁺ T cell isolation. A murine spleen was mashed using a cell strainer (Celltreat) on a 70 μm filter (ThermoFisher Scientific), incubated in RBC lysis buffer for min, and passed through a 40 μm filter (ThermoFisher Scientific). Cells were centrifuged at 500×g for 5 min in between steps. CD8⁺ T cells and CD4⁺ T cells were isolated using the CD8⁺ T Cell and CD4⁺ T Cell Isolation Kits (Miltenyi Biotec) according to the manufacturer's instructions. Purity and activation status were assessed by flow cytometry.

MHCI-restricted antigen cross-presentation and MHCII-restricted antigen presentation assays.

MHCI-restricted antigen cross presentation—Peritoneal macrophages or TAMs from E0771 tumors were seeded at a density of 100,000 cells/well (peritoneal macrophages and pooled TAM) or 200,000 cells/well (flow sorted TAMs) in tissue culture treated 96-well plates (Corning). For the OT-1 system, macrophages were incubated with OVA₂₅₇₋₂₆₄ peptide (10 μg/mL, InvivoGen®) or ovalbumin protein (OVA, 2 mg/mL, InvivoGen) for 2h. Cell surface MHCI bound OVA₂₅₇₋₂₆₄ signal was examined by staining cells with anti-OVA₂₅₇₋₂₆₄ (12-5743, ThermoFisher Scientific, 1:1000 dilution) For the pMel system, macrophages were incubated with gp10025-33 peptide (10 μM, Anaspec) or X-ray irradiated B16F10 cells (60 Gy, 50,000 cells) for 2 h. After two washes with PBS, CFSE-labeled CD8⁺ T cells isolated from OT-1 or pMel mice were added to each well (100,000/well) and co-cultured with macrophages for 72h. For antigen cross-presentation by TAMs from B16.0VA or B16F10 tumors, pooled TAMs or flow sorted M1-like and M2-like TAMs were directly co-cultured with CD8⁺ T cells isolated from OT-1 or pMel mice. For allostimulation, CD8⁺ T cells were co-cultured with TAMs that had not been pre-treated with antigens. For Anti-CD3 (5 μg/mL, 16-0033, ThermoFisher Scientific) and anti-CD28 (2 μg/mL, 16-0281, ThermoFisher Scientific) antibodies were used as a positive control.

MHCII-restricted antigen presentation by TAMS—TAMs were seeded at a density of 100,000 cells/well in tissue culture treated 96-well plates (Corning). For the OT-2 system, TAMs were incubated with OVA₃₃₂₋₃₃₉ peptide (10 μg/mL, InvivoGen) or ovalbumin protein (OVA, 2 mg/mL, InvivoGen) for 2h. For the TRP1 system, TAMs were incubated with TRP1₁₁₃₋₁₂₆ peptide (10 μg/mL, Biosynthesis) or X-ray irradiated B16F10 cells (60 Gy, 50,000 cells) for 2h. After two washes with PBS, CFSE-labeled CD4⁺ T cells isolated from OT-2 or TRP1 mice corresponding to each system were added to each well (100,000/well) and co-cultured with TAMs for 72h.

T cell activation—CD8⁺ or CD4⁺ T cells were treated with BD GolgiPlug for the final 6 h of coculture with macrophages to allow intracellular IFNγ accumulation. Cells were collected, washed in Stain Buffer (BD Biosciences) and stained for activation markers for 15 min in the dark at room temperature. Cells were fixed with BD Cytofix Fixation Buffer (BD Biosciences) for 20 min at 4° C. Fixed cells were permeabilized with BD Perm/Wash Buffer (BD Biosciences) and stained with anti-IFNγ (554413, BD Biosciences) and anti-CD44 (25-0441, ThermoFisher Scientific) antibodies. The percent of IFNγ⁺/CD44⁺CD8⁺ T cells was quantified by flow cytometry. In some cases, CD8⁺ T cell IFNγ production in the culture medium at 72h was quantified using a mouse IFN-γ ELISA kit (Invitrogen).

T cell proliferation—Isolated CD4⁺ or CD8⁺ T cells were labeled with 5 μM 5,6-carboxyfluorescein diacetate succinimidyl ester (CF SE, Invitrogen) according to the manufacturer's instructions. The number of proliferating cells (CF SE-diluted) was quantified using CountBright™ beads (Invitrogen). In some cases of T cell proliferation was quantified by the Proliferation Platform Software (FlowJo v.10.4.1).

Tumor inoculation and treatment. For the TNBC model, E0771 cells (0.5×10⁶) were injected into the 4^{th} mammary fat pad of the right ventral side of C57BL/6 mice. For other models, LLC1 cells (0.5×10⁶), B16F10 cells (1×10⁶), or B16. OVA cells (1×10⁶) were injected into the flank of C57BL/6 mice. Tumor volume was assessed by calipers, and experiments were terminated when tumor volume reached >−1000 mm 3. For in vivo treatments, 25 μg/injection of E64-DNA or DNA every 4 days, or 50 mg/kg/intraperitoneal injection of cyclophosphamide every other day for three doses, followed by a week rest and another three doses every other day (Sigma) was used.

Depletion of CD8⁺ T cells. Anti-mouse CD8a (BE0061, clone 2.43, Bio X Cell) or rat IgG2b (BE0086, clone MPC-11, Bio X Cell) were injected intraperitoneally (200 μg/injection) 3 days before the first treatment and once/week after the last treatment. CD8⁺ T cell depletion was confirmed by flow cytometry.

Depletion of TAMs. Anti-mouse CSF1R (BE0213, clone AFS98, Bio X Cell) or rat IgG2b (BE0086, clone MPC-11, Bio X Cell) were injected intraperitoneally (300 μg/injection) every other day for three doses before the first treatment, and every three days after the last treatment to maintain depletion.

Statistics. Statistical significance was determined with the Student's two-tailed, unpaired t-test. Linear regression was performed using Prism v.7 software. For shotgun proteomics studies, significance was assessed by a combination of the t-test and G-test (Becker, L. et al. A macrophage sterol-responsive network linked to atherogenesis. Cell Metab. 11, 125-135 (2010)) with correction for false-discovery rate (<5%) using PepC software (Heinecke, N. L., Pratt, B. S., Vaisar, T. & Becker, L. PepC: proteomics software for identifying differentially expressed proteins based on spectral counting. Bioinformatics 26, 1574-1575 (2010)).

Data availability. All data generated or analyzed during this study are included in this published article and its supplementary information files. Proteomics data are available via ProteomeXchange with identifier PXD028037.

### Results

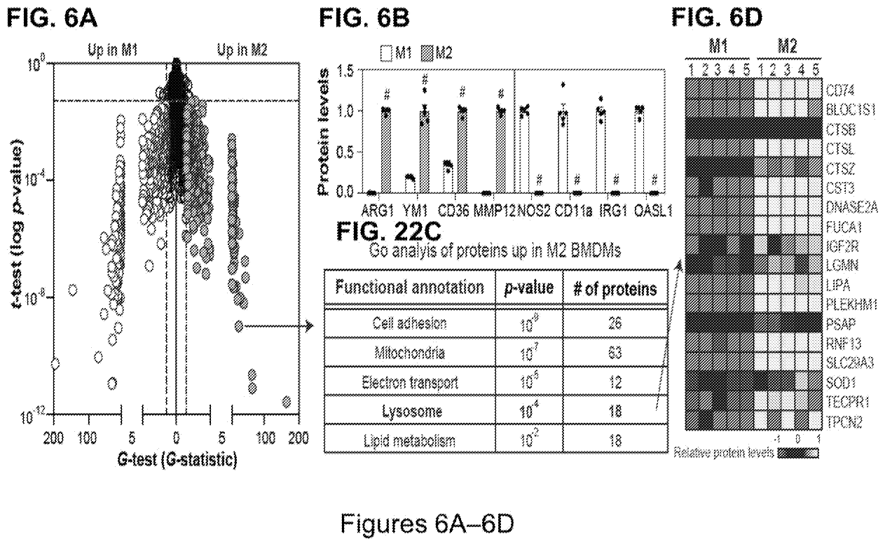

M2-like TAMs have elevated lysosomal proteins and activity. To identify tumor-promoting pathways in M2 macrophages, the cells were compared to anti-tumorigenic M1 macrophages. Shotgun proteomics analysis of cell lysates from M2-(IL-4) and M1-activated (LPS/IFNγ) bone marrow-derived macrophages (BMDMs) identified 337 and 413 proteins respectively that were significantly elevated (FDR<5%), many of which are previously described (e.g. M2: ARG1, YM1; M1: NOS2, CD11a) (Becker, L. et al. Unique proteomic signatures distinguish macrophages and dendritic cells. PLoS One 7, e33297 (2012)) (FIGS. 6A-6B).

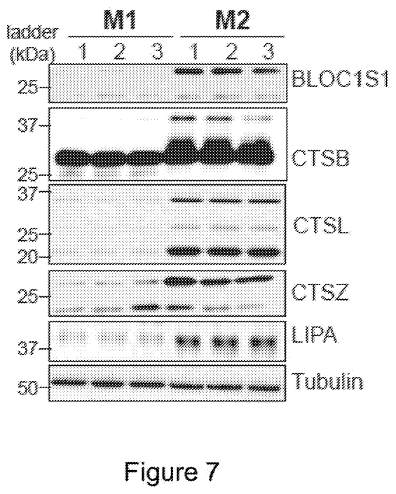

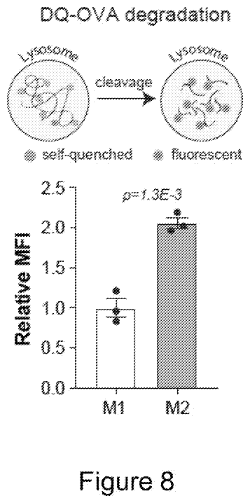

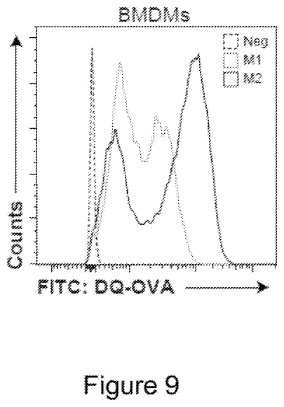

Bioinformatics analyses revealed enrichments in mitochondria, electron transport, and lipid metabolism M2 BMDMs (FIG. 6C), consistent with their reliance on oxidative phosphorylation (Odegaard, J. I. & Chawla, A. Alternative macrophage activation and metabolism. Annu. Rev. Pathol. 6, 275-297 (2011); Rodríguez-Prados, J.-C. et al. Substrate fate in activated macrophages: a comparison between innate, classic, and alternative activation. J. Immunol. 185, 605-614 (2010)). Interestingly, 18 lysosomal proteins were also enriched in M2 BMDMs (FIG. 6D), five of which were validated by immunoblotting (FIG. 7). Elevated lysosomal protein levels in M2 BMDMs were consistent with enhanced lysosomal degradation in an ovalbumin degradation assay (DQ-OVA) (FIG. 8, FIG. 9).

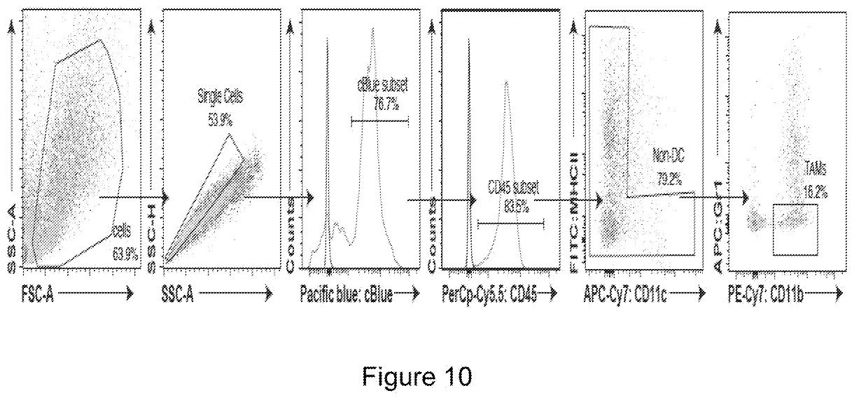

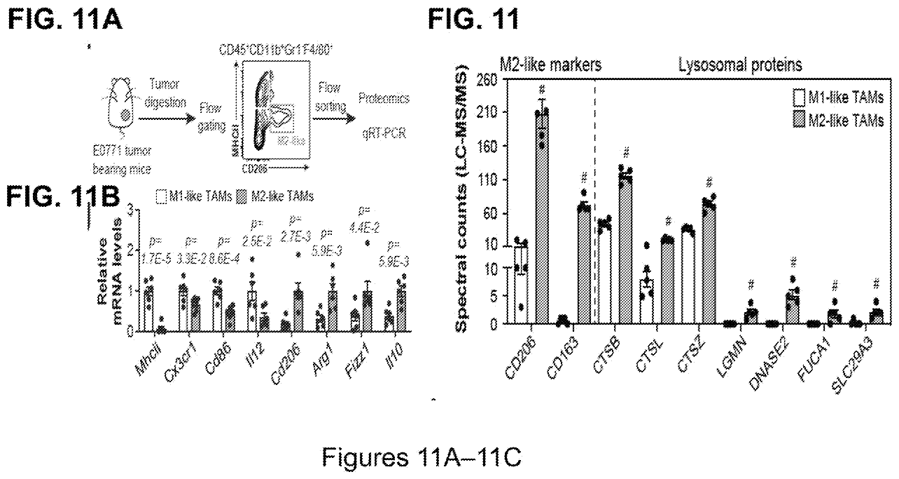

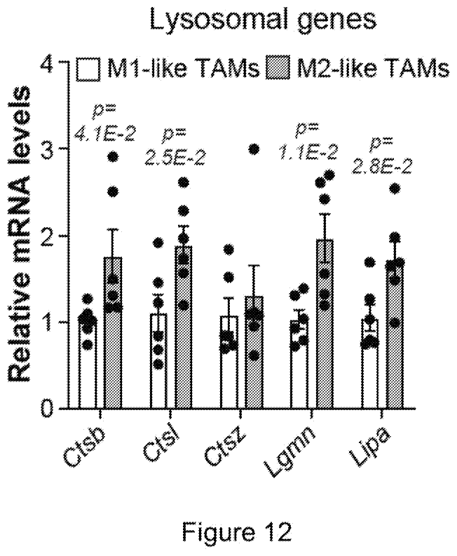

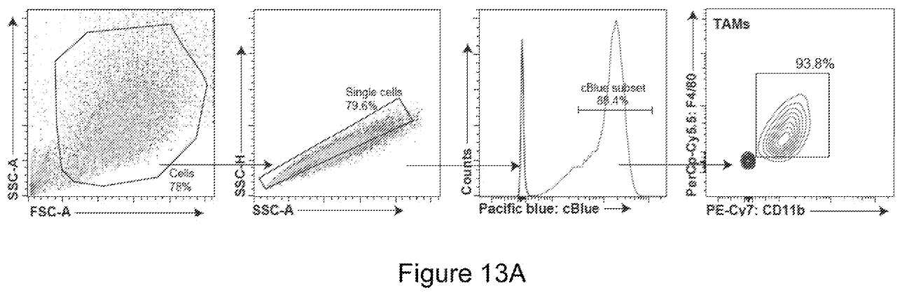

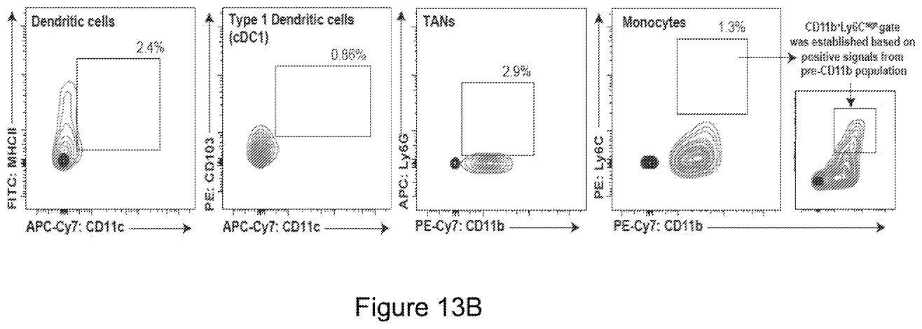

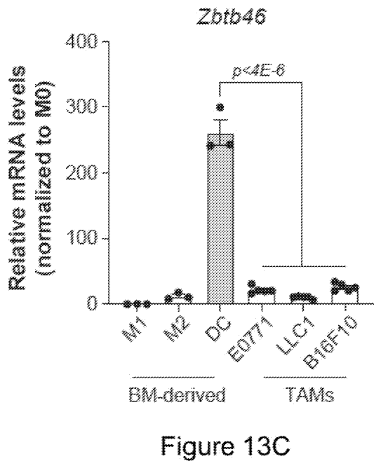

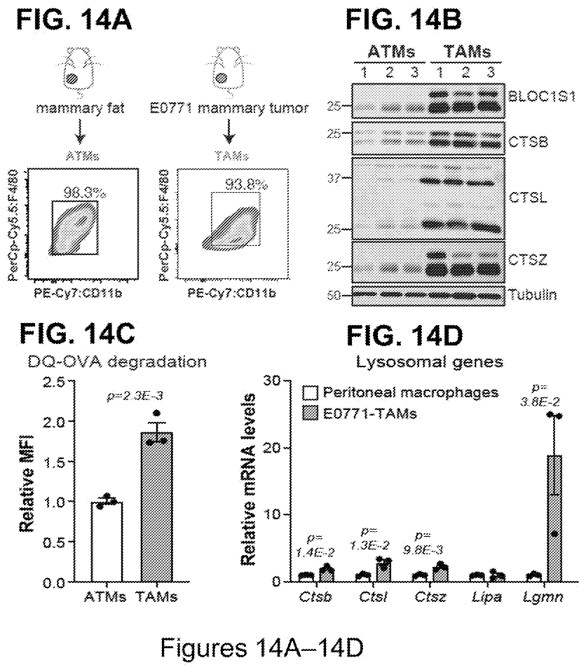

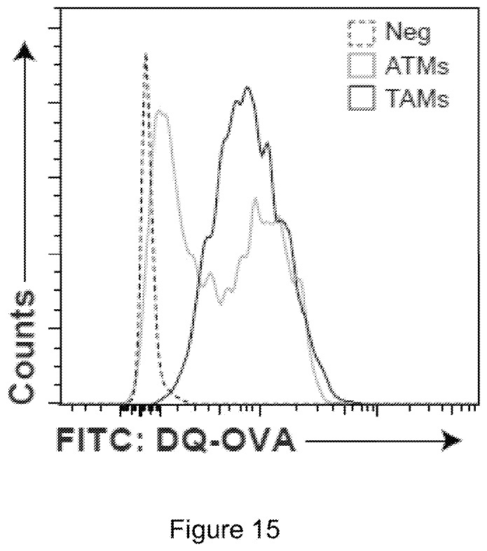

Because macrophages adopt more complex phenotypes in vivo (Geissmann, F., Gordon, S., Hume, D. A., Mowat, A. M. & Randolph, G. J. Unravelling mononuclear phagocyte heterogeneity. Nat. Rev. Immunol. 10, 453-460 (2010)), findings were tested in vivo by shotgun proteomics of M2-like (CD206^{high}MHCII^{low}) versus M1-like (CD206^{low}MHCII^{high}) TAMs (Xiong, H. et al. Anti-PD-L1 Treatment Results in Functional Remodeling of the Macrophage Compartment. Cancer Res. 79, 1493-1506 (2019); Lawrence, T. & Natoli, G. Transcriptional regulation of macrophage polarization: enabling diversity with identity. Nat. Rev. Immunol. 11, 750-761 (2011); Martinez, F. O. & Gordon, S. The M1 and M2 paradigm of macrophage activation: time for reassessment. F1000Prime Rep. 6, 13 (2014))(FIG. 10). Elevated lysosomal protein levels (p=10⁻³⁷) were observed in M2-like TAMs, many of which overlapped with those in M2 BMDMs (FIGS. 11A-11C). M2-like TAMs also showed elevated mRNA levels for these proteins (FIG. 12). Further, purified TAMs from E0771 tumors (FIGS. 13A-13C) showed elevated lysosomal enzyme levels/activity relative to mammary adipose tissue macrophages and thioglycolate-elicited peritoneal macrophages (FIGS. 14A-14D, FIG. 15).

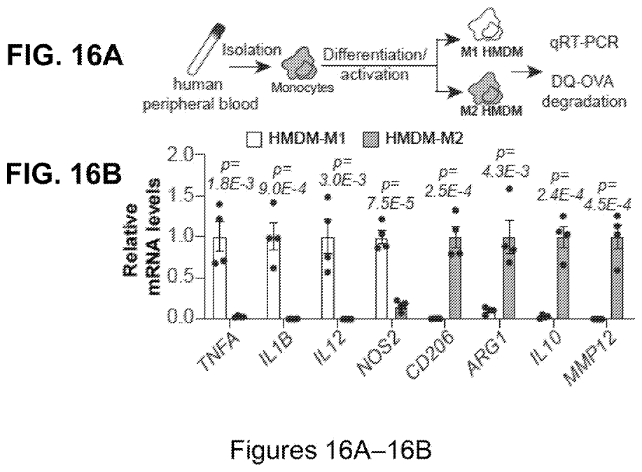

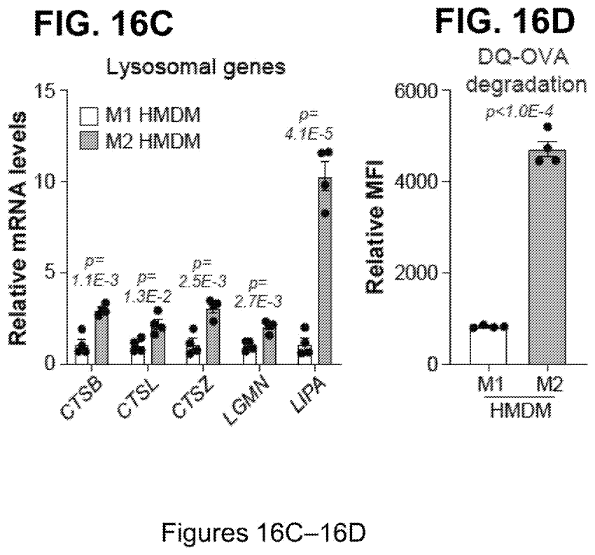

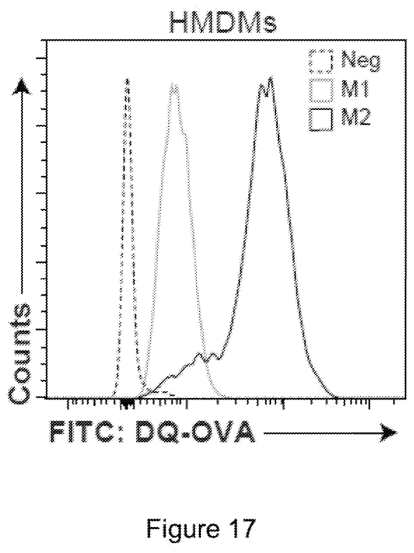

The regulation of lysosomal proteins and activity in human macrophages was explored, as these can exhibit distinct properties from their murine counterparts (Schroder, K. et al. Conservation and divergence in Toll-like receptor 4-regulated gene expression in primary human versus mouse macrophages. Proc. Natl. Acad. Sci. USA 109, E944-53 (2012); Thomas, A. C. & Mattila, J. T. “Of mice and men”: arginine metabolism in macrophages. Front. Immunol. 5, 479 (2014)). Compared to M1 human monocyte-derived macrophages (HMDMs), M2 HMDMs showed higher lysosomal gene expression and DQ-OVA degradation (FIGS. 16A-16D, FIG. 17). Analysis of TAMs from human ER+breast cancer patients further revealed an increase in DQ-OVA degradation in M2-like (CD206^{high}HLA-DR^{low}) versus M1-like (CD206^{low}HLA-DR^{high}) TAMs (FIGS. 18A-18B, FIG. 19, FIG. 20). These studies cumulatively demonstrate that lysosomal enzyme levels and/or activity are induced in M2-like macrophages in vitro and in vivo, in both mice and humans.

Reducing lysosomal proteins in TAMs promotes anti-tumor immunity. Next, the effect of reducing lysosomal activity on TAM function was explored. Several lysosomal proteins also showed elevated mRNA levels in M2 BMDMs suggesting transcriptional regulation (FIG. 12, FIG. 16C, FIG. 21). Further, mRNA levels, protein levels, and nuclear localization of transcription factor EB (TFEB), a master regulator of lysosome biogenesis, were also elevated (FIGS. 22A-22C)(Settembre, C. et al. TFEB links autophagy to lysosomal biogenesis. Science 332, 1429-1433 (2011); Sardiello, M. et al. A gene network regulating lysosomal biogenesis and function. Science 325, 473-477 (2009)). Tfeb was therefore knocked out in myeloid cells (mTfeb−/−) which lowered lysosomal gene expression and DQ-OVA degradation in both M2 BMDMs and TAMs (FIGS. 23A-23D, FIGS. 24A-24C, FIG. 25). Deleting Tfeb did not eliminate lysosomal gene expression or abolish degradation in M2 BMDMs and TAMs, but rather attenuated them to levels observed in M1 macrophages (FIG. 24B). This agrees with the current understanding that TFEB does not regulate basal lysosomal gene expression but rather induces expression in response to stimuli (Napolitano, G. & Ballabio, A. TFEB at a glance. J. Cell Sci. 129, 2475-2481 (2016)). Further, lysosome number, lysosomal pH, and autophagy were unaffected in TAMs from mTfeb−/− mice (FIGS. 26A-26C). Thus, mTfeb−/− reduced lysosomal protein levels and activity in M2-like TAMs while preserving basal lysosomal functions.

To test if the elevated lysosomal activity in TAMs contributes to tumorigenesis, mTfeb−/− mice and fl/fl littermate controls were injected with E0771 (triple-negative breast cancer), B16F10 (melanoma), or LLC1 (lung cancer) cells. Deleting Tfeb in myeloid cells attenuated tumor growth in all three models (FIG. 27, FIG. 28), implying that hyperactive lysosomes in TAMs promote tumor development.

Because TAMs promote tumor growth partly by suppressing adaptive immunity (Noy et al.; Mantovani et al.), tumor immune cells were quantified in mTfeb−/− and fl/fl control mice. Increases in total CD8⁺ T cells (CD3⁺CD4⁻CD8⁺) and effector CD8⁺ T cells (CD3⁺CD4⁻CD8⁺CD62L^{low}CD44^{high}) were observed in all 3 models. These changes were specific since TAMs (CD11b⁺F4/80⁺), tumor-associated neutrophils (TANs, CD11b⁺Ly6G⁺), DCs (CD11c⁺MHCII^{high}), and CD4⁺ T cells (CD3⁺CD4⁺CD8⁻) were minimally affected (FIG. 29, FIG. 30, FIGS. 31A-31B).

Next, experiments were conducted to test whether decreased tumor growth in mTfeb−/− mice relied on CD8⁺ T cells. Depleting CD8⁺ T cells restored tumor growth in m Tfeb−/− mice but not in fl/fl mice (FIG. 32, FIGS. 33A-33B). This suggests that lowering lysosomal activity in myeloid cells by deleting Tfeb activates CD8⁺ T cells, opposing tumorigenesis.

Deleting Tfeb could activate CD8⁺ T cells by inhibiting the M2-like phenotype of TAMs, which is linked to immune suppression in cancer (Noy et al.; Mantovani et al.). This possibility could be eliminated because M2 markers (Arg1, Il10, Fizz1) and M1 markers (Tnfa, 111b, Nos2) were minimally affected in TAMs from E0771, LLC1, and B16F10 tumors of mTfeb−/− versus fl/fl mice (FIG. 34).

Recent studies showed that TAMs cross-present antigens to activate class I restricted T cells (Singhal et al.). Moreover, in antigen-presenting cells, lysosomal proteolysis inversely correlates with their ability to present antigens (Delamarre, L., Pack, M., Chang, H., Mellman, I. & Trombetta, E. S. Differential lysosomal proteolysis in antigen-presenting cells determines antigen fate. Science 307, 1630-1634 (2005); Trombetta, E. S. & Mellman, I. Cell biology of antigen processing in vitro and in vivo. Annu. Rev. Immunol. 23, 975-1028 (2005)). Thus, deleting Tfeb could activate CD8⁺ T cells by enhancing antigen cross-presentation in TAMs. To test this, TAMs were isolated from B16.0VA tumors and co-cultured with CD8⁺ T cells from OT-1 or pMel mice to evaluate their antigen cross-presentation capability ex vivo (Lund, A. W. et al. VEGF-C promotes immune tolerance in B16 melanomas and cross-presentation of tumor antigen by lymph node lymphatics. Cell Rep. 1, 191-199 (2012)) (FIG. 35).

As in other models, B16.0VA tumor growth was attenuated in mTfeb−/− mice (FIG. 36). TAMs purified from mTfeb−/− mice activated OT-1 and pMel CD8⁺ T cells more effectively, consistent with increased IFNγ production and proliferation (FIGS. 37A-37B, FIGS. 38A-38B). Contamination with DCs, TANS and monocytes were ruled out by flow cytometric quantification of cell types and the expression levels of cell-specific transcription factors (Satpathy, A. T. et al. Zbtb46 expression distinguishes classical dendritic cells and their committed progenitors from other immune lineages. J. Exp. Med. 209, 1135-1152 (2012)) (FIGS. 13A-13C). Thus, genetically downregulating lysosomal activity in myeloid cells (via mTfeb−/−) attenuates tumor development by promoting adaptive immunity.

E64-DNA promotes antigen cross-presentation by TAMs. Because globally lowering lysosomal activity in TAMs improves antigen cross-presentation, it was desirable to identify a therapeutically actionable target. Bioinformatics analysis of the 18 lysosomal proteins elevated in M2 BMDMs (see FIG. 6D) pinpointed enrichments in antigen presentation and cysteine proteases, but not aspartic proteases (FIGS. 39A-39B). Moreover, cysteine protease levels and activity were elevated in M2-like TAMs in vivo (FIG. 11C, FIG. 40, FIG. 41), along with reduced antigen cross-presentation relative to M1-like TAMs (FIG. 42).

Unlike aspartic proteases, cysteine proteases fail to generate antigenic peptides when incubated with OVA in vitro and can completely digest OVA-derived antigenic peptides (Diment, S. Different roles for thiol and aspartyl proteases in antigen presentation of ovalbumin. J. Immunol. 145, 417-422 (1990); Rodriguez, G. M. & Diment, S. Destructive proteolysis by cysteine proteases in antigen presentation of ovalbumin. Eur. J. Immunol. 25, 1823-1827 (1995)). Incubating the antigenic peptide gp10025-33 with cysteine proteases (CTSB and CTSL) before delivering it to TAMs blocked their ability to activate CD8⁺ T cells, while incubation with aspartic proteases (CTSD and CTSE) did not (FIGS. 43A-43C). It was therefore hypothesized that elevated lysosomal cysteine protease activity in M2-like TAMs impedes antigen cross-presentation.

Treating TAMs with the small molecule cysteine protease inhibitor, E64, was considered (Matsumoto, K. et al. Structural basis of inhibition of cysteine proteases by E-64 and its derivatives. Biopolymers 51, 99-107 (1999)). However, E64 has difficulty penetrating cells (Powers, J. C., Asgian, J. L., Ekici, Ö. D. & James, K. E. Irreversible Inhibitors of Serine, Cysteine, and Threonine Proteases. Chem. Rev. 102, 4639-4750 (2002)), which could limit its access to the lysosome. With DNA nanotechnology one can localize diverse cargo, with tissue-specificity in lysosomes (Surana et al.; Chakraborty et al. 2017; Veetil et al., Chakraborty, K. et al. Tissue specific targeting of DNA nanodevices in a multicellular living organism. Elife 10, (2021)). One such pathway is endocytosis via scavenger receptors which are highly expressed in macrophages (Leung et al.). E64 was chemically conjugated to a 38-base pair DNA duplex to localize E64 to lysosomes of TAMs (FIG. 44). In the E64-DNA nanodevice, E64 is attached through a C6 amine linker to the 5′ end of one strand. The complementary strand displays an Alexa Fluor 647 dye to monitor cell-specificity and organelle localization (FIG. 45). The DNA scaffold enables cell-specific uptake by macrophages via scavenger receptors, localizes E64 specifically to lysosomes, and enables targeting specificity via the Alexa Fluor 647 moiety.

Indeed, E64-DNA localized specifically to lysosomes of TAMs to attenuate their capacity to degrade DQ-OVA, an effect that could not be reproduced with free E64 or free DNA (FIGS. 46A-46B, FIG. 47). E64-DNA uptake occurred via specific scavenger receptors because Scarb1−/− (scavenger receptor class B type 1) or Msr1−/− (macrophage scavenger receptor 1) reduced E64-DNA uptake by M2 BMDMs, while Cd36−/− (scavenger receptor class B, member 3) did not (FIG. 48). Different structural variants of E64-DNA were tested, namely ssDNA, dsDNA, ssRNA, and dsRNA, all 38 nucleotides long and tagged with Alexa 647. Internalization by M2 BMDMs required a ssDNA or dsDNA scaffold (FIGS. 49A-49B), suggesting that nanodevice uptake is specific for the DNA backbone and not simply on size or charge.

E64-DNA retained its specificity for cysteine proteases but did not impact cell viability, cysteine protease protein levels, or autophagy genes in TAMs (FIGS. 50A-50E). Importantly, E64-DNA did not activate the STING pathway (Burdette, D. L. & Vance, R. E. STING and the innate immune response to nucleic acids in the cytosol. Nat. Immunol. 14, 19-26 (2013)) as it did not induce TBK1 and IRF3 phosphorylation in TAMs, nor did it elevate inflammatory cytokine levels (FIGS. 51A-51B). This result was surprising, as in zebrafish brains, an immunogenic tag on the DNA scaffold is required to see an immune response in microglia (Veetil et al., DNA-based fluorescent probes of NOS2 activity in live brains, Proc Natl Acad Sci USA, 2020 Jun. 30; 117(26):14694-14702). 64-DNA did not alter the TAM phenotype given the unchanged M1- and M2-associated gene expression levels (FIG. 51B). Thus, E64-DNA attenuates lysosomal cysteine protease activity without significantly altering the TAM phenotype.

Next, the OVA-OT-1 CD8⁺ T cell system was used to evaluate if E64-DNA affected antigen cross-presentation by TAMs (FIG. 52). When TAMs were first treated with E64-DNA and then allowed to process OVA, they showed increased cell surface MHCI-associated OVA₂₅₇₋₂₆₄ as well as improved ability to induce CD8⁺ T cell IFNγ production and proliferation (FIGS. 53A-53C). Lysosomal processing was vital to antigen presentation because E64-DNA failed to further activate CD8⁺ T cells when TAMs were exposed to the antigenic OVA₂₅₇₋₂₆₄ peptide which directly binds WWI (FIGS. 53A-53C). Allostimulation was ruled out because the presence of an antigen was necessary (FIGS. 54A-54B). Treatment with E64 or DNA alone did not affect cross-presentation (FIGS. 53A-53C). Further the DQ-OVA degradation assay revealed attenuated cysteine protease activity indicating that E64-DNA targeted E64 to lysosomes (FIG. 43B).

Two approaches were used to evaluate the specificity of E64-DNA to antigen cross-presentation. First, aspartic proteases, another major class of lysosomal proteins, were inhibited to test whether this improved antigen cross-presentation. A DNA nanodevice bearing the aspartic protease inhibitor pepstatin A (PepA-DNA) had no effect on antigen cross-presentation by macrophages and a mild effect on tumor growth (FIGS. 55A-55G). Second, experiments were conducted to determine whether E64-DNA could improve MHCII-restricted antigen presentation. E64-DNA had no impact on WWII-restricted presentation by TAMs in the OVA-OT-2 CD4⁺ T cell and irradiated B16 (irrB16)-TRP-1 CD4⁺ T cell systems (FIGS. 56A-56F). These studies underscore a specific role for lysosomal cysteine proteases in antigen cross-presentation by TAMs and M2 macrophages.

E64-DNA preferentially targets M2-like TAMs. In D. rerio and C. elegans, DNA nanodevices target phagocytic cells that express scavenger receptors (Surana et al.; Veetil et al.), which are also elevated in murine macrophages (Canton, J., Neculai, D. & Grinstein, S. Scavenger receptors in homeostasis and immunity. Nat. Rev. Immunol. 13, 621-634 (2013)). Experiments were performed to test whether E64-DNA could preferentially target TAMs in mice by intratumoral (i.t.) injection into E0771 tumors (FIG. 57). E64-DNA (i.t.) was preferentially internalized by TAMs, where it specifically localized to lysosomes, attenuating DQ-OVA degradation (FIGS. 58A-58C, FIG. 59). Thus E64-DNA was targeted selectively to TAMs, and with organelle-level specificity, over other tumor cell types.

Approximately 80% of TAMs were labeled by E64-DNA (i.t.). Moreover, E64-DNA was ˜3-fold enriched in M2-like (CD206^{high}) relative to M1-like (CD206^{low}) TAMs in vivo (FIG. 60). A similar enrichment of E64-DNA labeling was observed in M2 over M1 BMDMs in vitro (FIGS. 61A-61C). This correlates well with the elevated expression of scavenger receptors in M2 versus M1 macrophages (Canton et al.), and also in M2-like versus M1-like TAMs from E0771 tumors (FIG. 62).

E64-DNA targets TAMs to promote anti-tumor immunity. High cysteine protease levels in tumors are poor prognostic markers for diverse solid tumors (Olson, 0. C. & Joyce, J. A. Cysteine cathepsin proteases: regulators of cancer progression and therapeutic response. Nat. Rev. Cancer 15, 712-729 (2015)). Activity-based probes revealed that tumor cysteine protease activity is largely TAM-associated (Gocheva, V. et al. IL-4 induces cathepsin protease activity in tumor-associated macrophages to promote cancer growth and invasion. Genes Dev. 24, 241-255 (2010), but how much of that is lysosomal is unknown. Interestingly, high doses of E64 (1 mg, daily) show limited impact on tumor growth in murine cancer models (Gopinathan, A. et al. Cathepsin B promotes the progression of pancreatic ductal adenocarcinoma in mice. Gut 61, 877-884 (2012)). This might be because the cell permeability of E64 is limited (Powers et al.), thereby reducing lysosomal access. Experiments were performed to test whether E64-DNA could overcome the cell-entry barrier and produce a therapeutic response.

E64-DNA at various doses (5-100 μs, single dose) was injected into E0771 tumors and found that TAMs internalized E64-DNA in a non-saturable, dose-dependent manner (FIG. 63). E64-DNA treatment attenuated DQ-OVA degradation by TAMs and diminished tumor growth, with both effects saturating at 25 μg (FIGS. 64A-64C), unlike free DNA and free E64 (FIG. 64C). Importantly, E64-DNA did not decrease E0771 proliferation in vitro (FIG. 65), revealing that its effect on tumor growth was not due to its action on cancer cells.

The efficacy of E64-DNA by intravenous (i.v.) delivery was then tested. E64-DNA (i.v.) was preferentially internalized by TAMs and attenuated their lysosomal activity as revealed by the DQ-OVA assay (FIGS. 66A-66B, FIG. 67). TAM labeling 7h post-injection was supported by in vitro serum stability studies, where −60% of E64-DNA remained intact at this time point (FIG. 68). Over 5-days, E64-DNA (i.v.) attenuated E0771 tumor growth (FIG. 69), increased CD8⁺ effector T cells in tumors (FIG. 70, FIG. 71), and increased markers of activation (4-1BB, OX40, CD69) and proliferation (Ki67 and BrdU) on CD8⁺ T cells (FIG. 72). These effects were not due to direct action of E64-DNA on CD8⁺ T cells (FIGS. 73A73B).

To test the importance of TAMs in E64-DNA-mediated tumor attenuation, an anti-CSF1R antibody was used to deplete TAMs in the E0771 model. Effects of E64-DNA on tumor growth and CD8⁺ effector T cells were both abolished in TAM-depleted mice (FIG. 74). Moreover, the abundance of CD8⁺ effector T cells inversely correlated with tumor volume in E64-DNA-treated mice, but not in DNA-treated or E64-DNA-treated mice depleted of TAMs (FIGS. 75A-75B).

These findings suggest a model wherein E64-DNA acts via TAMs to activate CD8⁺ T cells. Consistent with this model, depleting CD8⁺ T cells restored E0771 tumor growth in E64-DNA-treated mice (i.v.) but not in DNA-treated mice (FIG. 76A). CD8⁺ T cell function in DNA-treated mice (i.v.) could not be rescued by treatment with anti-PD-L1, which had no impact on tumor development (FIG. 76B). In contrast, treatment with anti-PD-L1 lessened tumor growth in mice treated with E64-DNA (i.v.) (FIG. 76B). Effects on CD8⁺ T cells were associated with improved cross-presentation by TAMs from E64-DNA-treated mice, in the E0771 (FIG. 77) and B16.0VA models, where E64-DNA (i.v.) also increased/activated CD8⁺ T cells and attenuated tumor growth (FIGS. 78A-78E).

Whether the improvement in antigen cross-presentation was specific to M2-like TAMs was further investigated. M1-like and M2-like TAMs were sorted from E0771 tumors, treated with E64-DNA ex vivo, and it was found that E64-DNA improved antigen cross-presentation by M2-like but not M1-like TAMs (FIG. 77). Collectively, these results suggest that reducing cysteine protease activity in lysosomes of M2-like TAMs activates CD8⁺ T cells and attenuates tumor growth.

E64-DNA-cyclophosphamide combination therapy regresses tumors. Although E64-DNA treatment attenuated tumor growth, it did not lead to sustained tumor regression as a monotherapy. Because E64-DNA enables TAMs to better utilize tumor antigens to activate CD8⁺ T cells, experiments were conducted to determine whether enhancing antigen supply by increasing the number of dead cancer cells could improve anti-tumor efficacy. The efficacy of cyclophosphamide (CTX), a frontline treatment for many cancers, in combination with E64-DNA was tested. CTX was delivered at metronomic doses (50 mg/kg/mice) to kill cancer cells and maintain anti-tumor immunity (Kerbel, R. S. & Kamen, B. A. The anti-angiogenic basis of metronomic chemotherapy. Nat. Rev. Cancer 4, 423-436 (2004); Sistigu, A. et al. Immunomodulatory effects of cyclophosphamide and implementations for vaccine design. Semin Immunopathol 33, 369-383 (2011)). Interestingly, combining E64-DNA (i.v.) with CTX led to sustained tumor regression in the E0771 model, an effect that could not be replicated by either treatment alone (FIG. 79).

### Conclusions

Although the pro-tumorigenic functions of TAMs are well known, TAMs can also be anti-tumorigenic (Mantovani et al.; Singhal et al.). Limited understanding of the underlying mechanisms has stymied the development of therapeutics that leverage their anti-tumor capabilities. Using discovery-based proteomics, it was shown that elevated activity of cysteine proteases in lysosomes of M2-like TAMs degrades tumor antigens and impedes antigen cross-presentation and CD8⁺ T cell activation in tumors (FIG. 80). This work supports the idea that the contribution of this pathway to adaptive immune suppression is governed by the abundance of M2-like TAMs, which is associated with poor prognosis across many cancers (Mantovani et al.; Gentles et al.; Takeya et al.).

Efficient antigen presentation requires optimal lysosomal activity since hypoactivity suppresses antigen generation while hyperactivity destroys them (Delamarre et al.; Trombetta et al.). It was shown that the lysosomal degradative capacity of macrophages is regulated by their activation state, wherein M2-like TAMs have heightened activity that limits antigen cross-presentation. Normally, M2-like macrophages clear dead host cells during wound repair (Murray, P. J. Macrophage Polarization. Annu. Rev. Physiol. 79, 541-566 (2017). Thus, enhanced proteolysis may help destroy antigens and prevent inadvertent adaptive immune activation, providing protection against potential autoimmune responses.

These studies demonstrated that the antigen destroying property of M2-like TAMs in tumors is detrimental as it limits CD8⁺ T cell activation. Indeed, while CD8⁺ T cells are present in E0771 tumors, they do not oppose tumor development unless mice are treated with E64-DNA, which attenuates lysosomal degradation in TAMs. These effects on TAMs are independent of changes to their M2-like phenotype. Thus, enabling antigen cross-presentation in M2-like TAMs facilitates adaptive immune activation even in an immunosuppressive environment. However, whether antigen cross-presentation by TAMs occurs locally in the tumor or in the tumor-draining lymph node, is yet to be determined.

Pre-clinical studies indicate several potential mechanisms by which cysteine proteases promote tumorigenesis, including cell intrinsic activity in multiple tumor cell types and extracellular activity that facilitates metastasis (Olson et al.). These studies revealed that suppressing lysosomal cysteine protease activity in TAMs impedes tumor development. This was achieved by linking a classical cysteine protease inhibitor, E64, to a lysosome-targeted DNA nanodevice. Not only did this strategy overcome the cell-permeability problems of E64, but the DNA nanodevice selectively targeted TAMs, localizing in their lysosomes and conferring therapeutic properties at doses of E64 that are otherwise ineffective.

The studies with E64-DNA have several important implications for implementing DNA nanodevices in therapeutics development. In contrast to aptamers, where DNA is the therapeutic (Pastor, F., Kolonias, D., McNamara, J. O. & Gilboa, E. Targeting 4-1BB costimulation to disseminated tumor lesions with bi-specific oligonucleotide aptamers. Mol. Ther. 19, 1878-1886 (2011); Siegers, G. M. et al. Anti-leukemia activity of in vitro-expanded human gamma delta T cells in a xenogeneic Ph+leukemia model. PLoS One 6, e16700 (2011)), this approach uses DNA as a carrier to specifically target the therapeutic to macrophages via scavenger receptors (MSR1, SCARB1). Unlike DNA nanostructures that deliver therapeutics such as doxorubicin, siRNA, or thrombin, that cause the death of the target cells (Cho, Y., Lee, J. B. & Hong, J. Controlled release of an anti-cancer drug from DNA structured nano-films. Sci. Rep. 4, 4078 (2014); Lee, H. et al. Molecularly self-assembled nucleic acid nanoparticles for targeted in vivo siRNA delivery. Nat. Nanotechnol. 7, 389-393 (2012); Li, S. et al. A DNA nanorobot functions as a cancer therapeutic in response to a molecular trigger in vivo. Nat. Biotechnol. 36, 258-264 (2018); Li, Z., He, X., Luo, X., Wang, L. & Ma, N. DNA-Programmed Quantum Dot Polymerization for Ultrasensitive Molecular Imaging of Cancer Cells. Anal. Chem. 88, 9355-9358 (2016); Zhang, P. et al. Near Infrared-Guided Smart Nanocarriers for MicroRNA-Controlled Release of Doxorubicin/siRNA with Intracellular ATP as Fuel. ACS Nano 10, 3637-3647 (2016)), the approach does not eliminate the target. Instead, it reprograms an organelle to endow it a new, therapeutically beneficial property. Finally, this approach facilitates the intracellular delivery of a therapeutic to lysosomes of macrophages. Polymer-based or liposome-based nanoparticles that are phagocytosed, can also reach the lysosome. However, it has proven challenging for nanoparticles to specifically target macrophages over other phagocytes (Gustafson, H. H., Holt-Casper, D., Grainger, D. W. & Ghandehari, H. Nanoparticle uptake: the phagocyte problem. Nano Today 10, 487-510 (2015); Kelly, C., Jefferies, C. & Cryan, S.-A. Targeted liposomal drug delivery to monocytes and macrophages. J. Drug Deliv. 2011, 727241 (2011)).

An advantage of using DNA-based lysosomal intervention over genetic strategies to suppress lysosomal activity (ie. TFEB siRNA) is the lower cell-type specificity. In vivo delivered siRNA, even using other nanoparticle-carriers, lacks the macrophage-specificity obtained with E64-DNA. This is particularly important when targeting TFEB, because of its involvement in diverse physiological processes in many cell types e.g., global Tfeb−/− mouse is not viable (Napolitano et al.).

In summary, these studies demonstrated the therapeutic value of targeting a DNA nanodevice with organelle-level precision in TAMs within murine tumors. Successful localization of the nanodevice in lysosomes reprograms TAMs to improve their ability to present antigens, which in turn, activates the adaptive immune response. The new-found capability of organelle-targeted DNA nanodevices to modulate macrophage behavior in tumors suggests the broader possibility of manipulating macrophage function in other diseases, because every organ harbors tissue-specific macrophages of variable phenotype.

### Example 6: In Vivo Testing of DNA-Derivatized LXR Agonist in Atherosclerotic Mice

### Introduction

To assess the efficacy of the treatment and indirect effects of hepatocyte response, atherosclerotic mice were tested for effects on both atherosclerotic lesions and triglyceride levels.

### Methods

Male LDL receptor deficient mice were fed an atherogenic diet (Envigo® TD96121) for 10 weeks. After 6 weeks of diet feeding the mice were injected 5 days a week with either 50 μg double stranded DNA (n=10) or 50 μg DNA with T090137 attached to the end of both strands (1.9 μg T090137/mouse/day; n=10). After 4 weeks of injection, the mice were perfusion fixed with 4% paraformaldehyde and the heart and upper vasculature embedded in OCT. Sections of the innominate artery and aortic root were stained with Oil Red O and lesion area quantitated.

An additional set of animals were perfused with cold sterile phosphate buffered saline after 3 weeks of injection. The atherosclerotic lesions were dissected out of the upper vasculature and aortic root. Total RNA isolated from the dissected lesions were analyzed by quantitative real time PCR.

### Results

T0901317-DNA lessens atherosclerotic lesions without inducing hyperglyceridemia (FIGS. 81A-81C).

### Conclusions

Derivatizing LXR agonists to nucleic targeting modules, thereby targeting them to macrophages, is a viable approach for treating atherosclerosis and avoiding hyperglyceridemia associated with LXR agonist treatment.

### Example 7: Additional DNA Drug Conjugation Studies

### Introduction

Additional studies were performed using nucleic acid-derivatized therapeutics, including a LDHA inhibitor ((R)-GNE-140), a BTK inhibitor (Ibrutinib), and an LXR agonist (GW3965).

### Methods

Oligonucleotides. All fluorescently labeled and unlabeled DNA oligonucleotides were HPLC-purified and obtained from IDT (Coralville, IA, USA).

Preparation of oligonucleotide samples. All oligonucleotides were dissolved in Milli-Q water, aliquoted as a 100 μM stock for sequence variation studies and −500 μM for drug conjugations and applications. Concentration of each oligonucleotide was measured using UV absorbance at 260 nm and oligo aliquots and stored at −20° C.

For sequence variation studies a 10 μM sample was prepared by mixing 10 μM of D1 and D2 in equimolar ratios in 20 mM potassium phosphate buffer, pH 7.4 containing 100 mM KCl. The resulting solution was heated to 90° C. for 5 min, cooled to the room temperature at 5° C./15 mins and equilibrated at 4° C. overnight.

For drug conjugation studies a 100 μM sample was prepared by mixing 100 μM of D1 and D2 in equimolar ratios in 20 mM potassium phosphate buffer, pH 7.4 containing 100 mM KCl. A maximum of 100 μL per sample was annealed and for preps which required more; multiple annealing reactions were set up simultaneously. The resulting solution was heated to 90° C. for 5 min, cooled to the room temperature at 5° C./15 mins and equilibrated at 4° C. overnight. The solutions were then pooled together to set up a single conjugation reaction.

Amide Based Conjugations.

E64 (S7379, Selleckchem), GW3965 (HY-10627A, Medchem Express), were conjugated to a 38mer double stranded DNA containing an amine modification at the 5′ end of one of the strands (usually D1).

Drug molecule (5 equivalents) was added to of N-Hydroxysuccinimide (25 equivalents, 130672, Sigma) and N-(3-Dimethylaminopropyl)-N′-ethylcarbodiimide hydrochloride (E7750, Sigma) in a maximum volume of 50 μL reaction in 10 mM IVIES buffer pH 5.5 solution for 1 hour at room temperature. After an hour 25 μL of the reaction is added to the solution of amine modified DNA (pH 7.4) while the remaining solution is stored at −20C until further use.

After ˜10-12 hours the remaining 25 μL of activated drug solution is added to the DNA solution. The reaction is continued further for another 8 hours after which the sample is stored at −20C until further purification.

For purification, the reaction mixture is subjected to a 3 k cut of based amicon filtration based on the manufacturers' protocols. Amicon based centrifugation is performed 8-10 times to remove maximum amount of small molecule reactants. The drug DNA conjugate is then stored at −20° C. until further use.

Azide Based Conjugations.

Bioconjugatable version of Ibrutinib (PF-06658607; Sigma) is conjugated to DNA containing an azide modification at the 5′ end of one of the strands (usually D1) via click chemistry. Briefly, 10 equivalents excess of drug molecule were added to dsDNA solution (20 mM, pH 7.4) in presence of 1 mM Tris(2-carboxyethyl)phosphine (TCEP, Thermofisher), 200 1..LM Tris[(1-benzyl-1H-1,2,3-triazol-4-yl)methyl]amine (TBTA, Sigma-Aldrich) and 1 mM CuSO₄. The reaction was left at room temperature for 16 hours following which an amicon purification was perform as mentioned above.

DBCO Based Conjugations.

T0901317 (HY-10626, Medchem Express) and (R)-GNE-140 (HY-100742A, MedchemExpress), were converted into azide containing molecules by conjugation to azido acetic acid (1081, Click Chemistry Tools). These azido molecules were then conjugated to a 38mer double stranded DNA containing a DBCO modification at the 5′ end of one of the strands (usually D1) via copper free click chemistry.

4 equivalents of T0901317 were added to 1 equivalent of azidoacetic acid in presence of 1 equivalent of 4-(Dimethylamino)pyridine (DMAP, Sigma Aldrich). 2 equivalents of N,N-Dicyclohexylcarbodiimide (DCC, Sigma Aldrich) was added in DCM (10 mL) at 0° C. The reaction was then stirred at room temperature for 10 hours. Urea was filtered out at the end of the reaction and the product formation was confirmed by mass spectrometry.

2 equivalents of (R)-GNE-140 were added to 1 equivalent of azidoacetic acid in presence of 2 equivalent of Oxalyl chloride (Sigma Aldrich) and 2 equivalents of N,N-Dimethylformamide (DMF, Sigma Aldrich) at 0° C. in DCM. The product formation was confirmed by mass spectrometry.

The azido molecules were then conjugated to DBCO containing DNA (5 equivalents excess) in 20 mM phosphate buffer, pH 7.4. The reaction was left overnight following which an amicon based purification protocol was performed.

Isolation and activation of bone marrow-derived macrophage (BMDM).

BMDMs were differentiated from bone marrow stem cells with L-cell conditioned media for six days as previously described (Kratz et al.).

Murine adipose tissue macrophage (ATM) isolation.

Adipose tissue was digested with Type 1 Collagenase (Worthington, 1 mg/mL) at 37° C. with shaking at 160 RPM for 45 mins. Digested tissue was filtered through a 100 μm cell strainer, incubated in RBC lysis buffer for 5 min, and passed through a 40 μm cell strainer. ATMs were isolated using CD11b microbeads (Miltenyi Biotec) as previously described (Kratz et al.).

DNA-Drug Conjugate Treatments on Cells.

Indicated concentrations of drugs, DNA drug conjugates were added to BMDMs in L-cell conditioned media and ATMs in RPMI supplemented with heat inactivated FBS and Penstrep.

Measurement of Gene Expression by qRT-PCR.

Cell pellets were lysed in RLT buffer, total RNA was isolated using the RNAeasy kit (Qiagen) with on-the-column DNAse digestion (Qiagen), converted to cDNA using reverse transcription kit (Qiagen), and amplified using QuantiTect SYBR Green PCR Kits (Qiagen). The following murine primers were used:

18s forward:
(SEQ ID NO: 47)
GCCGCTAGAGGTGAAATTCTT,
reverse:
(SEQ ID NO: 48)
CGTCTTCGAACCTCCGACT.
Tnfa forward:
(SEQ ID NO: 14)
CACCACGCTCTTCTGTCTACTG,
reverse:
(SEQ ID NO: 15)
GCTACAGGCTTGTCACTCGAA.
Il1b forward:
(SEQ ID NO: 16)
AACTCAACTGTGAAATGCCACC,
reverse:
(SEQ ID NO: 17)
CATCAGGACAGCCCAGGTC.
Nos2 forward:
(SEQ ID NO: 18)
GCTCCTCTTCCAAGGTGCTT,
reverse:
(SEQ ID NO: 19)
TTCCATGCTAATGCGAAAGG.
Arg1 forward:
(SEQ ID NO: 20)
CTCCAAGCCAAAGTCCTTAGAG,
reverse:
(SEQ ID NO: 21)
AGGAGCTGTCATTAGGGACATC.
Il10 forward:
(SEQ ID NO: 49)
GCTCTTACTGACTGGCATGAG,
reverse:
(SEQ ID NO: 50)
CGCAGCTCTAGGAGCATGTG.
Srebp2 forward:
(SEQ ID NO: 85)
GTTGACCACGCTGAAGACAGA,
reverse:
(SEQ ID NO: 86)
CACCAGGGTTGGCACTTGAA
Abca1 forward:
(SEQ ID NO: 87)
GCTTGTTGGCCTCAGTTAAGG,
reverse:
(SEQ ID NO: 88)
GTAGCTCAGGCGTACAGAGAT
Cd36 forward:
(SEQ ID NO: 89)
ATGGGCTGTGATCGGAACTG,
reverse:
(SEQ ID NO: 90)
GTCTTCCCAATAAGCATGTCTCC
Abcg1 forward:
(SEQ ID NO: 91)
GTGGATGAGGTTGAGACAGACC,
reverse:
(SEQ ID NO: 92)
CCTCGGGTACAGAGTAGGAAAG
Lxra forward:
(SEQ ID NO: 93)
ACAGAGCTTCGTCCACAAAAG,
reverse:
(SEQ ID NO: 94)
GCGTGCTCCCTTGATGACA
ApoE forward:
(SEQ ID NO: 95)
CGCAGGTAATCCCAGAAGC,
reverse:
(SEQ ID NO: 96)
CTGACAGGATGCCTAGCCG
Ppary forward:
(SEQ ID NO: 97)
GGAAGACCACTCGCATTCCTT,
reverse:
(SEQ ID NO: 98)
GTAATCAGCAACCATTGGGTCA
Plin2 forward:
(SEQ ID NO: 99)
ACTCCACCCACGAGACATAGA,
reverse:
(SEQ ID NO: 100)
AAGAGCCAGGAGACCATTTC

### Results

In vitro testing revealed that GNE-DNA attenuates hypoxia-induced lactate production by macrophages (FIGS. 82 and 83). Furthermore, ibrutinib-DNA attenuates inflammation in adipose tissue macrophages (ATMs) from obese mice and changes the expression profile of several genes involved in inflammation of metabolically active macrophages (MMe) (FIG. 84). Finally, GW3965-DNA enhances lipid metabolism gene expression in macrophages (FIG. 85).

### Conclusions

These results provide a strong proof of concept that delivery of nucleic-derivatized therapeutic agents to the lysosome of macrophages enables activation/inhibition of cytosolic drug targets. Therefore, it is believed that nucleic acid-derivatized therapeutics represent a powerful new tool for the treatment of a variety of disease states (FIG. 86).

### Example 8: DNA Labeling Studies

### Introduction

Studies were performed using nucleic acid-derivatized magnetic labeling agents (e.g., contrast agents) to determine their effectiveness as Mill imaging agents.

### Methods

Establishing Nucleic Acid-Derivatized Magnetic Labeling Agents.

Initially, dsDNA targeting modules were labelled with an Alexa 647 fluorophore, with some of the targeting modules further labeled with either an iron oxide labeling agent (at a 10 nm concentration, “Probe 1”) or a gadolinium labeling agent (“Probe 3”) at 100 nM each. To test that the addition of either magnetic labeling agent did not effect macrophage uptake of the devices, BMDMs were labelled with a negative control (no targeting module), a dsDNA targeting module without a magnetic label, Probe 1, or Probe 3 (100 nM), and mean fluorescence intensity of Alexa 647 was measured by flow cytometry.

Ex Vivo Labeling of Tumors.

To determine that there was not perturbation of the Mill agents post conjugation, ex vivo E0771 tumors were injected with either Probe 1 (40 μM) or Probe 3 (20 μM) and imaged by MRI.

In Vivo Labeling of Tumors.

Female C57BL/6 mice were injected with 0.5×10\hat{ }6 E0771 cells into the right mammary gland. When the tumor reached 150 mm\hat{ }3, the mouse was injected intravenously with 200 μg double stranded DNA with gadolinium attached to both strands (12.6 μg gadolinium). Ten 1 mm MRI slices of the lower abdomen were obtained over 4 hours.

In Vivo Labeling of Atherosclerotic Lesions.

Male LDL receptor deficient mice fed an atherogenic diet (Envigo TD96121) for 4 months were intravenously injected with 200 μg double stranded DNA with gadolinium attached to both strands (12.6 μg gadolinium). Fifteen 1 mm MRI slices of the abdomen at the level of the kidneys were obtained over 1 hour. Time of flight was used to confirm location of arteries.

### Results

As shown in FIG. 87 (upper panel), labeling of the targeting modules with magnetic labels did not significantly affect macrophage uptake (measured by MFI of Alexa 647) of the magnetically-labelled devices. FIGS. 88A-88B demonstrate that the MRI agents were readily viewable in ex vivo tumor samples and therefore not perturbed by conjugation to the dsDNA targeting modules. Arrows point to darker regions which were the injection sites showing MM agents (greyish-black regions).

The arrows in the T1 map indicate uptake of gadolinium-DNA (Probe 3) into the tumor (FIG. 89). Orientation on horizontal axis is abdomen to back side. Orientation on vertical axis is moving towards the tail. The left image in FIG. 89 shows a strong water signal in the tumor (and bladder), which after 2h post IV injection shifts to a gadolinium signal (maximally in the bladder, indicating renal clearance). Strong gadolinium signal is still evident in the tumor 4h post injection (right image).

FIGS. 90A-90B show the time course of accumulation of the Probe 3 signal intratumorally over time after DNA complex injection. Signal maximum was reached by 20 min and remained stable.

FIG. 91 shows a gradient echo anatomy reference (left image) revealing the location of the kidneys (arrows) and the dynamic contrast enhanced MM image of the same slice (right image) demonstrates uptake of the gadolinium-DNA in the atherosclerotic lesion in the descending artery in the renal area (bright region marked by the arrow).

### Conclusions

These results provide a strong proof of concept that delivery of nucleic-derivatized Mill imaging agents via intravenous administration can be used for imaging of tumors and atherosclerotic lesions in vivo. Therefore, it is believed that nucleic acid-derivatized MRI imaging agents also represent a powerful new tool for imaging and monitoring the status of targeted disease sites. It is further envisioned that dually functional devices that combine a therapeutic module and a labelling module could be used to both treat and monitor treatment progression of tumors and artherosclerotic lesions via MM imaging or other imaging means.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

SEQUENCES

| SEQ ID | | |
|---|---|---|
| NO: | Name | Sequence |
| 1 | mTfeb-/- forward | GTAGAACTGAGTCAAGGCATACTGG |
| 2 | mTfeb-/- reverse | GGGTCCTACCTACCACAGAGC |
| 3 | loxp-R | CTTCGTATAATGTATGCTATACGAAG |
| 4 | Ctsb forward | CTGCGCGGGTATTAGGAGT |
| 5 | Ctsb reverse | CAGGCAAGAAAGAAGGATCAAG |
| 6 | Cstl forward | AGACCGGCAAACTGATCTCA |
| 7 | Cstl reverse | ATCCACGAACCCTGTGTCAT |
| 8 | Ctsz forward | GGCCAGACTTGCTACCATCC |
| 9 | Ctsz reverse | ACACCGTTCACATTTCTCCAG |
| 10 | Lipa forward | CTGGTGAGGAACACTCGGTC |
| 11 | Lipa reverse | AGCCGTGCTGAAGATACACAA |
| 12 | Lgmn forward | ATTCCTGACGAGCAGATCATAGT |
| 13 | Lgmn reverse | GTGCCGTTAGGTCGGTTGA |
| 14 | Tnfa forward | CACCACGCTCTTCTGTCTACTG |
| 15 | Tnfa reverse | GCTACAGGCTTGTCACTCGAA |
| 16 | Ilb forward | AACTCAACTGTGAAATGCCACC |
| 17 | Ilb reverse | CATCAGGACAGCCCAGGTC |
| 18 | Nos2 forward | GCTCCTCTTCCAAGGTGCTT |
| 19 | Nos2 reverse | TTCCATGCTAATGCGAAAGG |
| 20 | Arg1 forward | CTCCAAGCCAAAGTCCTTAGAG |
| 21 | Arg1 reverse | AGGAGCTGTCATTAGGGACATC |
| 22 | Ym1 forward | GCCCACCAGGAAAGTACACA |
| 23 | Ym1 reverse | TGTTGTCCTTGAGCCACTGA |
| 24 | Fizz1 forward | CCTGCTGGGATGACTG |
| 25 | Fizz1 reverse | TGGGTTCTCCACCTCTTCAT |
| 26 | Gapdh forward | TGGCCTTCCGTGTTCCTAC |
| 27 | Gapdh reverse | GAGTTGCTGTTGAAGTCGCA |
| 28 | Cd11b forward | CCATGACCTTCCAAGAGAATGC |
| 29 | Cd11b reverse | ACCGGCTTGTGCTGTAGTC |
| 30 | Sqstm1 forward | GAGTAACACTCAGCCAAGCA |
| 31 | Sqstm1 reverse | TTCACCTGTAGATGGGTCCA |
| 32 | Map1lc3b forward | TTGCAGCTCAATGCTAACCA |
| 33 | Mapllc3b reverse | GGCATAAACCATGTACAGGA |
| 34 | Vps11 forward | AAAAGAGAGACGGTGGCAATC |
| 35 | Vps11 reverse | AGCCCAGTAACGGGATAGTTG |
| 36 | Uvrag forward | CTGACAGAAAAGGAGCGAGA |
| 37 | Uvrag reverse | GGATGGCATTGGAGATGTGA |
| 38 | Atg9b forward | CCATCCCACAATGATACACACC |
| 39 | Atg9b reverse | CCTCTAGCCGTTCATAGTCCT |
| 40 | D1 | NH₂ATCAACACTGCACACCAGACAGCAAGATC |
| | | CTATATATA |
| 41 | D2-A647, ssDNA | Alexa647TATATATAGGATCTTGCTGTCTGGTGT |
| | | GCAGTGTTGAT |
| 42 | D2 | TATATATAGGATCTTGCTGTCTGGTGTGCAGTG |
| | | TTGAT |
| 43 | SSRNA, dsRNA 1 | Alexa647UAUAUAUAGGAUCUUGCUGUCUGGU |
| | | GUGCAGUGUUGAU |
| 44 | dsRNA 2 | AUCAACACUGCACACCAGACAGCAAGAUCCU |
| | | AUAUAUA |
| 45 | RNA:DNA hybrid 1 | Alexa647TATATATAGGATCTTGCTGTCTGGTGT |
| | | GCAGTGTTGAT |
| 46 | RNA: DNA hybrid 2 | AUCAACACUGCACACCAGACAGCAAGAUCCU |
| | | AUAUAUA |
| 47 | Mouse 18s forward | GCCGCTAGAGGTGAAATTCTT |
| 48 | Mouse 18s reverse | CGTCTTCGAACCTCCGACT |
| 49 | Il10 forward | GCTCTTACTGACTGGCATGAG |
| 50 | Il10 reverse | CGCAGCTCTAGGAGCATGTG |
| 51 | Vps18 forward | AGTACGAGGACTCATTGTCCC |
| 52 | Vps18 reverse | TGGGCACTTACATACCCAGAAT |
| 53 | Becn1 forward | AGGTACCGACTTGTTCCCTA |
| 54 | Becn1 reverse | TCCATCCTGTACGGAAGACA |
| 55 | Tfeb forward | CAAGGAGCGGCAGAAGAAAG |
| 56 | Tfeb reverse | GCTGCTTGTTGTCATCTCC |
| 57 | Human 18s forward | CCCAACTTCTTAGAGGGACAAG |
| 58 | Human 18s reverse | CATCTAAGGGCATCACAGACC |
| 59 | Human CTSB forward | GAGCTGGTCAACTATGTCAACA |
| 60 | Human CTSB reverse | GCTCATGTCCACGTTGTAGAAGT |
| 61 | Human CTSL forward | AAACTGGGAGGCTTATCTCACT |
| 62 | Human CTSL reverse | GCATAATCCATTAGGCCACCAT |
| 63 | Human CTSZ forward | ACCAATGTGGGACATGCAATG |
| 64 | Human CTSZ reverse | TTGCGTAGATTTCTGCCATCA |
| 65 | Human LIPA forward | CCCACGTTTGCACTCATGTC |
| 66 | Human LIPA reverse | CCCAGTCAAAGGCTTGAAACTT |
| 67 | Human LGMN forward | TCCGGCAAAGTCCTGAAGAG |
| 68 | Human LGMN reverse | GGCAGCAGTAGTTGCATAAACA |
| 69 | Human TNFA forward | CAGCCTCTTCTCCTTCCTGAT |
| 70 | Human TNFA reverse | GCCAGAGGGCTGATTAGAGA |
| 71 | Human IL1B forward | TCTGTACCTGTCCTGCGTGT |
| 72 | Human IL1B reverse | ACTGGGCAGACTCAAATTCC |
| 73 | Human IL12 forward | GCGGAGCTGCTACACTCTC |
| 74 | Human IL12 reverse | CCATGACCTCAATGGGCAGAC |
| 75 | Human NOS2 forward | CAGCGGGATGACTTTCCAAG |
| 76 | Human NOS2 reverse | AGGCAAGATTTGGACCTGCA |
| 77 | Human CD206 forward | GGCGGTGACCTCACAAGTAT |
| 78 | Human CD206 reverse | ACGAAGCCATTTGGTAAACG |
| 79 | Human ARG1 forward | GGCAAGGTGATGGAAGAAAC |
| 80 | Human ARG1 reverse | AGTCCGAAACAAGCCAAGGT |
| 81 | Human IL10 forward | GGGAGAACCTGAAGACCCTC |
| 82 | Human IL10 reverse | ATAGAGTCGCCACCCTGATG |
| 83 | Human MMP12 forward | CATGAACCGTGAGGATGTTGA |
| 84 | Human MMP12 reverse | GCATGGGCTAGGATTCCACC |
| 85 | Srebp2 forward | GTTGACCACGCTGAAGACAGA |
| 86 | Srebp2 reverse | CACCAGGGTTGGCACTTGAA |
| 87 | Abca1 forward | GCTTGTTGGCCTCAGTTAAGG |
| 88 | Abca1 reverse | GTAGCTCAGGCGTACAGAGAT |
| 89 | Cd36 forward | ATGGGCTGTGATCGGAACTG |
| 90 | Cd36 reverse | GTCTTCCCAATAAGCATGTCTCC |
| 91 | Abcg1 forward | GTGGATGAGGTTGAGACAGACC |
| 92 | Abcg1 reverse | CCTCGGGTACAGAGTAGGAAAG |
| 93 | Lxra forward | ACAGAGCTTCGTCCACAAAAG |
| 94 | Lxra reverse | GCGTGCTCCCTTGATGACA |
| 95 | ApoE forward | CGCAGGTAATCCCAGAAGC |
| 96 | ApoE reverse | CTGACAGGATGCCTAGCCG |
| 97 | Ppary forward | GGAAGACCACTCGCATTCCTT |
| 98 | Ppary reverse | GTAATCAGCAACCATTGGGTCA |
| 99 | Plin2 forward | ACTCCACCCACGAGACATAGA |
| 100 | Plin2 reverse | AAGAGCCAGGAGACCATTTC |

1. A composition, comprising:
a nucleic acid targeting module; and
a therapeutic agent attached to the nucleic acid targeting module,
wherein the nucleic acid targeting module targets the therapeutic agent to a lysosome of a macrophage.

2. The composition of claim 1, wherein the therapeutic agent is covalently attached to the nucleic acid targeting module.

3. The composition of claim 1, wherein the nucleic acid targeting module comprises single stranded deoxyribose nucleic acid (ssDNA), double-stranded DNA (dsDNA), modified DNA, single stranded ribonucleic acid (ssRNA), double-stranded RNA (dsRNA), modified RNA, and/or a RNA/DNA complex.

4. The composition of claim 3, wherein the nucleic acid targeting module is a double-stranded DNA molecule.

5. The composition of claim 3, wherein the nucleic acid targeting module is 38 base pairs in length.

6. The composition of claim 1, wherein the nucleic acid targeting module comprises a first single-stranded nucleic acid molecule and a second single-stranded nucleic acid molecule that is partially or fully complementary to the first single-stranded molecule.

7. The composition of claim 6, wherein each of the first and second single-stranded nucleic acid molecules is between 15 and 500 nucleotides in length.

8. The composition of claim 6, wherein each of the first and second single-stranded nucleic acid molecules is between 30 and 50 nucleotides in length.

9. The composition of claim 6, wherein the first single-stranded nucleic acid molecule comprises the nucleic acid sequence of SEQ ID NO: 40, and wherein the second single-stranded nucleic acid molecule comprises the nucleic acid sequence of SEQ ID NO: 41 or SEQ ID NO: 42.

10-11. (canceled)

12. The composition of claim 1, wherein the therapeutic agent comprises a small molecule or a peptide.

13. (canceled)

14. The composition of claim 1, wherein the therapeutic agent comprises a cathepsin inhibitor, a LDHA inhibitor, a neoantigen, a BTK inhibitor, a SYK inhibitor, and/or an LXR agonist.

15. The composition of claim 14, wherein the cathepsin inhibitor is a cysteine protease inhibitor or an aspartic protease inhibitor.

16. The composition of claim 15, wherein the cysteine protease inhibitor is E64.

17. The composition of claim 15, wherein the aspartic protease inhibitor is CA074 and/or pepstatin A.

18. The composition of claim 14, wherein the LDHA inhibitor is FX11, gossypol, GSK2837808A, (R)-GNE-140, galloflavin, NHI-2, and/or machilin.

19. The composition of claim 14, wherein the BTK inhibitor is ibrutinib.

20. The composition of claim 14, wherein the LXR agonist is GW3965 and/or T0901317.

21. The composition of claim 1 further comprising a labeling module optionally attached to the nucleic acid targeting module and/or the therapeutic agent.

22. The composition of claim 21, wherein the labeling module comprises one or more of a fluorescent agent, a chemiluminescent agent, a chromogenic agent, a quenching agent, a radionucleotide, an enzyme, a substrate, a cofactor, an inhibitor, a nanoparticle, and a magnetic particle.

23. (canceled)

24. A method of treating or preventing cancer in a subject in need thereof, comprising:
administering to the subject a composition, the composition comprising
a nucleic acid targeting module, and
one or more therapeutic agents, wherein the nucleic acid targeting module targets the one or more therapeutic agents to a lysosome of a tumor associated macrophage (TAM).

25-39. (canceled)

40. A method of treating obesity, diabetes, and/or insulin resistance in a subject in need thereof, comprising:
administering to the subject a composition, the composition comprising
a nucleic acid targeting module, and
one or more therapeutic agents attached to the nucleic acid targeting module,
wherein the nucleic acid targeting module targets the one or more therapeutic agents to a lysosome of a macrophage.

41-43. (canceled)

44. A method of treating atherosclerosis in a subject in need thereof, comprising:
administering to the subject a composition, the composition comprising
a nucleic acid targeting module, and
an LXR agonist attached to the nucleic acid targeting module,
wherein the nucleic acid targeting module targets the LXR agonist to the lysosome of a macrophage.

45. The composition of claim 1 further comprising
a secondary therapeutic agent.

46. The composition of claim 45, wherein the secondary therapeutic agent is an immune checkpoint inhibitor and/or an anticancer agent.

47-58. (canceled)

59. A method of administering a therapeutic agent to a subject, comprising:
a) providing a therapeutic construct comprising a therapeutic agent attached to a nucleic acid targeting module, wherein the nucleic acid targeting module targets the therapeutic agent to a lysosome of a macrophage; and
b) administering the therapeutic construct to the subject.

60-61. (canceled)

62. A method of minimizing a side-effect of a therapeutic agent, comprising:
administering to a subject in need thereof a therapeutic agent attached to a nucleic acid targeting module, wherein the nucleic acid targeting module targets the therapeutic agent to a lysosome of a macrophage,
wherein the therapeutic agent is released from the lysosome of the macrophage upon degradation of the targeting module,
wherein the therapeutic agent is released into the cytosol, nucleus, and/or immediate extracellular microenvironment of the macrophage to minimize the side-effect of the therapeutic agent that occurs when the therapeutic agent is administered systemically.

63-64. (canceled)

65. A method of sensitizing a subject to a therapy, comprising:
a) administering to a subject in need thereof a therapeutic construct comprising a therapeutic agent attached to a nucleic acid targeting module, wherein the nucleic acid targeting module targets the therapeutic agent to a lysosome of a macrophage; and
b) administering to the subject the therapy to which the subject is to be sensitized,
wherein the therapeutic construct sensitizes the subject to the therapy.

66-70. (canceled)

71. A composition, comprising:
a nucleic acid targeting module; and
a labeling module attached to the nucleic acid targeting module,
wherein the nucleic acid targeting module targets the labeling module to a lysosome of a macrophage.

72-74. (canceled)

75. A method of administering a labeling module to a subject, comprising:
a) providing a labeling construct comprising a labeling module attached to a nucleic acid targeting module, wherein the nucleic acid targeting module targets the labeling construct to a lysosome of a macrophage; and
b) administering the labeling construct to the subject.

76. A method, comprising:
administering to a subject a labeling construct comprising a labeling module attached to a nucleic acid targeting module,
wherein the nucleic acid targeting module targets the labeling module to a lysosome of a macrophage.

77. A method of imaging a biological phenomenon in a subject, comprising:
a) administering to a subject a labeling construct comprising a labeling module attached to a nucleic acid targeting module, wherein the nucleic acid targeting module targets the labeling module to a lysosome of a macrophage; and
b) detecting the labeling module.

78-81. (canceled)

82. A method of imaging a biological phenomenon associated with obesity, diabetes, and/or insulin in a subject in need thereof, comprising:
administering to the subject a composition, the composition comprising
a nucleic acid targeting module, and
one or more labeling modules attached to the nucleic acid targeting module,
wherein the nucleic acid targeting module targets the one or more labeling modules to a lysosome of a macrophage.

83-85. (canceled)


SEQUENCE LISTING (100 sequences)

SEQ ID NO: 1 - DNA, 25 bp, Artificial Sequence
  mTfeb-/- forward
gtagaactgagtcaaggcatactgg

SEQ ID NO: 2 - DNA, 21 bp, Artificial Sequence
  mTfeb-/- reverse
gggtcctacctaccacagagc

SEQ ID NO: 3 - DNA, 26 bp, Artificial Sequence
  loxp-R
cttcgtataatgtatgctatacgaag

SEQ ID NO: 4 - DNA, 19 bp, Artificial Sequence
  Ctsb forward
ctgcgcgggtattaggagt

SEQ ID NO: 5 - DNA, 22 bp, Artificial Sequence
  Ctsb reverse
caggcaagaaagaaggatcaag

SEQ ID NO: 6 - DNA, 20 bp, Artificial Sequence
  Cstl forward
agaccggcaaactgatctca

SEQ ID NO: 7 - DNA, 20 bp, Artificial Sequence
  Cstl reverse
atccacgaaccctgtgtcat

SEQ ID NO: 8 - DNA, 20 bp, Artificial Sequence
  Ctsz forward
ggccagacttgctaccatcc

SEQ ID NO: 9 - DNA, 21 bp, Artificial Sequence
  Ctsz reverse
acaccgttcacatttctccag

SEQ ID NO: 10 - DNA, 20 bp, Artificial Sequence
  Lipa forward
ctggtgaggaacactcggtc

SEQ ID NO: 11 - DNA, 21 bp, Artificial Sequence
  Lipa reverse
agccgtgctgaagatacacaa

SEQ ID NO: 12 - DNA, 23 bp, Artificial Sequence
  Lgmn forward
attcctgacgagcagatcatagt

SEQ ID NO: 13 - DNA, 19 bp, Artificial Sequence
  Lgmn reverse
gtgccgttaggtcggttga

SEQ ID NO: 14 - DNA, 22 bp, Artificial Sequence
  Tnfa forward
caccacgctcttctgtctactg

SEQ ID NO: 15 - DNA, 21 bp, Artificial Sequence
  Tnfa reverse
gctacaggcttgtcactcgaa

SEQ ID NO: 16 - DNA, 22 bp, Artificial Sequence
  Ilb forward
aactcaactgtgaaatgccacc

SEQ ID NO: 17 - DNA, 19 bp, Artificial Sequence
  Ilb reverse
catcaggacagcccaggtc

SEQ ID NO: 18 - DNA, 20 bp, Artificial Sequence
  Nos2 forward
gctcctcttccaaggtgctt

SEQ ID NO: 19 - DNA, 20 bp, Artificial Sequence
  Nos2 reverse
ttccatgctaatgcgaaagg

SEQ ID NO: 20 - DNA, 22 bp, Artificial Sequence
  Arg1 forward
ctccaagccaaagtccttagag

SEQ ID NO: 21 - DNA, 22 bp, Artificial Sequence
  Arg1 reverse
aggagctgtcattagggacatc

SEQ ID NO: 22 - DNA, 20 bp, Artificial Sequence
  Ym1 forward
gcccaccaggaaagtacaca

SEQ ID NO: 23 - DNA, 20 bp, Artificial Sequence
  Ym1 reverse
tgttgtccttgagccactga

SEQ ID NO: 24 - DNA, 16 bp, Artificial Sequence
  Fizz1 forward
cctgctgggatgactg

SEQ ID NO: 25 - DNA, 20 bp, Artificial Sequence
  Fizz1 reverse
tgggttctccacctcttcat

SEQ ID NO: 26 - DNA, 19 bp, Artificial Sequence
  Gapdh forward
tggccttccgtgttcctac

SEQ ID NO: 27 - DNA, 20 bp, Artificial Sequence
  Gapdh reverse
gagttgctgttgaagtcgca

SEQ ID NO: 28 - DNA, 22 bp, Artificial Sequence
  Cd11b forward
ccatgaccttccaagagaatgc

SEQ ID NO: 29 - DNA, 19 bp, Artificial Sequence
  Cd11b reverse
accggcttgtgctgtagtc

SEQ ID NO: 30 - DNA, 20 bp, Artificial Sequence
  Sqstm1 forward
gagtaacactcagccaagca

SEQ ID NO: 31 - DNA, 20 bp, Artificial Sequence
  Sqstm1 reverse
ttcacctgtagatgggtcca

SEQ ID NO: 32 - DNA, 20 bp, Artificial Sequence
  Map1lc3b forward
ttgcagctcaatgctaacca

SEQ ID NO: 33 - DNA, 20 bp, Artificial Sequence
  Map1lc3b reverse
ggcataaaccatgtacagga

SEQ ID NO: 34 - DNA, 21 bp, Artificial Sequence
  Vps11 forward
aaaagagagacggtggcaatc

SEQ ID NO: 35 - DNA, 21 bp, Artificial Sequence
  Vps11 reverse
agcccagtaacgggatagttg

SEQ ID NO: 36 - DNA, 20 bp, Artificial Sequence
  Uvrag forward
ctgacagaaaaggagcgaga

SEQ ID NO: 37 - DNA, 20 bp, Artificial Sequence
  Uvrag reverse
ggatggcattggagatgtga

SEQ ID NO: 38 - DNA, 22 bp, Artificial Sequence
  Atg9b forward
ccatcccacaatgatacacacc

SEQ ID NO: 39 - DNA, 21 bp, Artificial Sequence
  Atg9b reverse
cctctagccgttcatagtcct

SEQ ID NO: 40 - DNA, 38 bp, Artificial Sequence
  D1
  misc_feature (1)..(1): 5′-NH2
atcaacactgcacaccagacagcaagatcctatatata

SEQ ID NO: 41 - DNA, 38 bp, Artificial Sequence
  D2-A647, ssDNA
  misc_feature (1)..(1): 5′-Alexa647
tatatataggatcttgctgtctggtgtgcagtgttgat

SEQ ID NO: 42 - DNA, 38 bp, Artificial Sequence; identical to SEQ ID NO: 41
  D2

SEQ ID NO: 43 - RNA, 38 nt, Artificial Sequence
  ssRNA, dsRNA 1
  misc_feature (1)..(1): 5′-Alexa647
uauauauaggaucuugcugucuggugugcaguguugau

SEQ ID NO: 44 - RNA, 38 nt, Artificial Sequence
  dsRNA 2
aucaacacugcacaccagacagcaagauccuauauaua

SEQ ID NO: 45 - DNA, 38 bp, Artificial Sequence; identical to SEQ ID NO: 41
  RNADNA hybrid 1
  misc_feature (1)..(1): 5′-Alexa647

SEQ ID NO: 46 - DNA, 38 bp, Artificial Sequence
  RNA DNA hybrid 2
aucaacacugcacaccagacagcaagauccuauauaua

SEQ ID NO: 47 - DNA, 21 bp, Artificial Sequence
  Mouse 18s forward
gccgctagaggtgaaattctt

SEQ ID NO: 48 - DNA, 19 bp, Artificial Sequence
  Mouse 18s reverse
cgtcttcgaacctccgact

SEQ ID NO: 49 - DNA, 21 bp, Artificial Sequence
  Il10 forward
gctcttactgactggcatgag

SEQ ID NO: 50 - DNA, 20 bp, Artificial Sequence
  Il10 reverse
cgcagctctaggagcatgtg

SEQ ID NO: 51 - DNA, 21 bp, Artificial Sequence
  Vps18 forward
agtacgaggactcattgtccc

SEQ ID NO: 52 - DNA, 22 bp, Artificial Sequence
  Vps18 reverse
tgggcacttacatacccagaat

SEQ ID NO: 53 - DNA, 20 bp, Artificial Sequence
  Becn1 forward
aggtaccgacttgttcccta

SEQ ID NO: 54 - DNA, 20 bp, Artificial Sequence
  Becn1 reverse
tccatcctgtacggaagaca

SEQ ID NO: 55 - DNA, 20 bp, Artificial Sequence
  Tfeb forward
caaggagcggcagaagaaag

SEQ ID NO: 56 - DNA, 19 bp, Artificial Sequence
  Tfeb reverse
gctgcttgttgtcatctcc

SEQ ID NO: 57 - DNA, 22 bp, Artificial Sequence
  Human 18s forward
cccaacttcttagagggacaag

SEQ ID NO: 58 - DNA, 21 bp, Artificial Sequence
  Human 18s reverse
catctaagggcatcacagacc

SEQ ID NO: 59 - DNA, 22 bp, Artificial Sequence
  Human CTSB forward
gagctggtcaactatgtcaaca

SEQ ID NO: 60 - DNA, 23 bp, Artificial Sequence
  Human CTSB reverse
gctcatgtccacgttgtagaagt

SEQ ID NO: 61 - DNA, 22 bp, Artificial Sequence
  Human CTSL forward
aaactgggaggcttatctcact

SEQ ID NO: 62 - DNA, 22 bp, Artificial Sequence
  Human CTSL reverse
gcataatccattaggccaccat

SEQ ID NO: 63 - DNA, 21 bp, Artificial Sequence
  Human CTSZ forward
accaatgtgggacatgcaatg

SEQ ID NO: 64 - DNA, 21 bp, Artificial Sequence
  Human CTSZ reverse
ttgcgtagatttctgccatca

SEQ ID NO: 65 - DNA, 20 bp, Artificial Sequence
  Human LIPA forward
cccacgtttgcactcatgtc

SEQ ID NO: 66 - DNA, 22 bp, Artificial Sequence
  Human LIPA reverse
cccagtcaaaggcttgaaactt

SEQ ID NO: 67 - DNA, 20 bp, Artificial Sequence
  Human LGMN forward
tccggcaaagtcctgaagag

SEQ ID NO: 68 - DNA, 22 bp, Artificial Sequence
  Human LGMN reverse
ggcagcagtagttgcataaaca

SEQ ID NO: 69 - DNA, 21 bp, Artificial Sequence
  Human TNFA forward
cagcctcttctccttcctgat

SEQ ID NO: 70 - DNA, 20 bp, Artificial Sequence
  Human TNFA reverse
gccagagggctgattagaga

SEQ ID NO: 71 - DNA, 20 bp, Artificial Sequence
  Human IL1B forward
tctgtacctgtcctgcgtgt

SEQ ID NO: 72 - DNA, 20 bp, Artificial Sequence
  Human IL1B reverse
actgggcagactcaaattcc

SEQ ID NO: 73 - DNA, 19 bp, Artificial Sequence
  Human IL12 forward
gcggagctgctacactctc

SEQ ID NO: 74 - DNA, 21 bp, Artificial Sequence
  Human IL12 reverse
ccatgacctcaatgggcagac

SEQ ID NO: 75 - DNA, 20 bp, Artificial Sequence
  Human NOS2 forward
cagcgggatgactttccaag

SEQ ID NO: 76 - DNA, 20 bp, Artificial Sequence
  Human NOS2 reverse
aggcaagatttggacctgca

SEQ ID NO: 77 - DNA, 20 bp, Artificial Sequence
  Human CD206 forward
ggcggtgacctcacaagtat

SEQ ID NO: 78 - DNA, 20 bp, Artificial Sequence
  Human CD206 reverse
acgaagccatttggtaaacg

SEQ ID NO: 79 - DNA, 20 bp, Artificial Sequence
  Human ARG1 forward
ggcaaggtgatggaagaaac

SEQ ID NO: 80 - DNA, 20 bp, Artificial Sequence
  Human ARG1 reverse
agtccgaaacaagccaaggt

SEQ ID NO: 81 - DNA, 20 bp, Artificial Sequence
  Human IL10 forward
gggagaacctgaagaccctc

SEQ ID NO: 82 - DNA, 20 bp, Artificial Sequence
  Human IL10 reverse
atagagtcgccaccctgatg

SEQ ID NO: 83 - DNA, 21 bp, Artificial Sequence
  Human MMP12 forward
catgaaccgtgaggatgttga

SEQ ID NO: 84 - DNA, 20 bp, Artificial Sequence
  Human MMP12 reverse
gcatgggctaggattccacc

SEQ ID NO: 85 - DNA, 21 bp, Artificial Sequence
  Srebp2 forward
gttgaccacgctgaagacaga

SEQ ID NO: 86 - DNA, 20 bp, Artificial Sequence
  Srebp2 reverse
caccagggttggcacttgaa

SEQ ID NO: 87 - DNA, 21 bp, Artificial Sequence
  Abca1 forward
gcttgttggcctcagttaagg

SEQ ID NO: 88 - DNA, 21 bp, Artificial Sequence
  Abca1 reverse
gtagctcaggcgtacagagat

SEQ ID NO: 89 - DNA, 20 bp, Artificial Sequence
  Cd36 forward
atgggctgtgatcggaactg

SEQ ID NO: 90 - DNA, 23 bp, Artificial Sequence
  Cd36 reverse
gtcttcccaataagcatgtctcc

SEQ ID NO: 91 - DNA, 22 bp, Artificial Sequence
  Abcg1 forward
gtggatgaggttgagacagacc

SEQ ID NO: 92 - DNA, 22 bp, Artificial Sequence
  Abcg1 reverse
cctcgggtacagagtaggaaag

SEQ ID NO: 93 - DNA, 21 bp, Artificial Sequence
  Lxra forward
acagagcttcgtccacaaaag

SEQ ID NO: 94 - DNA, 19 bp, Artificial Sequence
  Lxra reverse
gcgtgctcccttgatgaca

SEQ ID NO: 95 - DNA, 19 bp, Artificial Sequence
  ApoE forward
cgcaggtaatcccagaagc

SEQ ID NO: 96 - DNA, 19 bp, Artificial Sequence
  ApoE reverse
ctgacaggatgcctagccg

SEQ ID NO: 97 - DNA, 21 bp, Artificial Sequence
  Ppary forward
ggaagaccactcgcattcctt

SEQ ID NO: 98 - DNA, 22 bp, Artificial Sequence
  Ppary reverse
gtaatcagcaaccattgggtca

SEQ ID NO: 99 - DNA, 21 bp, Artificial Sequence
  Plin2 forward
actccacccacgagacataga

SEQ ID NO: 100 - DNA, 20 bp, Artificial Sequence
  Plin2 reverse
aagagccaggagaccatttc